(19) United States
(12) Patent Application Publication
(10) Pub. No.: US 2023/0303502 A1
(43) Pub. Date: Sep. 28, 2023

(54) HETEROARYL AND HETEROCYCLYL COMPOUNDS

(71) Applicant: SIGILON THERAPEUTICS, INC., Cambridge, MA (US)

(72) Inventors: Richard Heidebrecht, Somerville, MA (US); Christopher P. Hencken, Boston, MA (US); Omar De Paolis, Boston, MA (US); Weiheng Wang, Bedford, MA (US); Matthew Buchanan, Atkinson, NH (US)

(21) Appl. No.: 18/019,664

(22) PCT Filed: Aug. 4, 2021
(86) PCT No.: PCT/US2021/044564

§ 371 Date: Feb. 3, 2023

Related U.S. Application Data
(60) Provisional application No. 63/060,968, filed on Aug. 4, 2020.

(51) Int. Cl. C07D 249/04; C07D 471/04; C07D 487/04; C07D 401/12; C07D 403/12; C07D 403/14

(52) U.S. Cl. CPC C07D 249/04; C07D 471/04; C07D 487/04; C07D 401/12; C07D 403/12; C07D 403/14

(57) ABSTRACT

Described herein are compounds of Formulas (I), (II), and (III) and pharmaceutically acceptable salts thereof, related compositions and preparations thereof, as well as methods of making and using the same.

### CLAIM OF PRIORITY

This application claims priority to U.S. Application No. 63/060,968, filed Aug. 4, 2020, the disclosure of which is incorporated herein by reference in its entirety.

### BACKGROUND

Certain heteroaryl and heterocyclyl compounds exhibit low onset temperatures and may present a safety hazard, e.g., under laboratory conditions. As such, there is a need in the art for new methods to prepare these heteroaryl and heterocyclyl compounds, as well as related intermediates.

### SUMMARY

Described herein are compounds of Formulas (I), (II), and (III) and pharmaceutically acceptable salts thereof, related compositions and preparations thereof, as well as methods of making and using the same. The present disclosure also features compositions and preparations of compounds of Formulas (I), (II), or (III) comprising a second agent, such as a compound of Formula (IV), e.g., at an amount less than about 5%, 2.5%, or 1% of the total composition. In particular, the compounds of Formulas (I), (II), and (III) and related compositions and preparations may be useful for the synthesis of afibrotic polymers, which, inter alia, may reduce the foreign body response in a subject or diminish pericapsular fibrotic overgrowth (PFO) on an object implanted in or delivered to the subject. Methods of making and using the compounds, preparations, and compositions are also described herein.

Current methods for preparing afibrotic polymers require the use of intermediates (e.g., azide compounds) that are highly reactive and may exhibit unfavorable energetic profiles. For example, certain afibrotic polymers comprising internal triazole moieties are currently prepared using intermediates with low onset temperatures (e.g., an onset temperature less than, e.g., 150° C.). These intermediates may pose an explosion hazard when working in low pressure conditions in the laboratory, particularly in the large quantities required for commercial scale up. Accordingly, improved synthetic methods for preparation of certain afibrotic polymers were sought that entail energetically favorable intermediates, e.g., that exhibit higher onset temperatures.

In one aspect, the present disclosure features a compound of Formula (I):

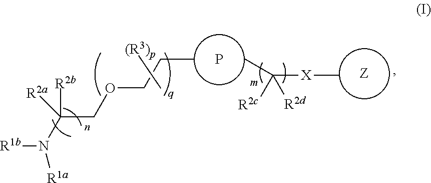

or a pharmaceutically acceptable salt thereof, wherein Ring P is heterocyclyl or heteroaryl, each of which is optionally substituted with 1-6 R⁴; Ring Z is cycloalkyl, heterocyclyl, aryl, or heteroaryl, each of which is optionally substituted with 1-6 R⁵; X is O, S, N(R^{A}), C₁-C₁₂ alkylene, C₁-C₁₂ alkenylene, C₂-C₁₂ heteroalkylene, or absent; Ria is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group; R^{1b} is an amine-protecting group; or R^{1a} and R^{1b} may be taken together to form a cycloalkyl, heterocyclyl, aryl, or heteroaryl ring, each of which is optionally substituted with 1-6 R⁶; each of R^{2a}, R^{2b}, R^{2c}, and R^{2d} is independently hydrogen, C₁-C₁₂ alkyl, C₂-C₁₂ heteroalkyl, halogen, or OR^{C}; or R^{2a} and R^{2b} or R^{2c} and R^{2d} are taken together to form an oxo; each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B}); each R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B}); each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl; m and n are each independently 0, 1, 2, 3, 4, 5, or 6; p is 0, 1, 2, 3, 4, or 5; and q is an integer from 0 to 25. In an embodiment, the amine protecting group comprises an acid-labile amine-protecting group or a base-labile amine-protecting group. In an embodiment, the amine-protecting group is selected from tert-butyloxycarbonyl (Boc), 9-fluorenylmethoxycarbonyl (Fmoc), benzyl (Bn), allyl (Al), nitrobenzenesulfonyl (Nosyl), dithiolan-2-imine, and trifluoroacetyl.

In another aspect, the present disclosure features a compound of Formula (II):

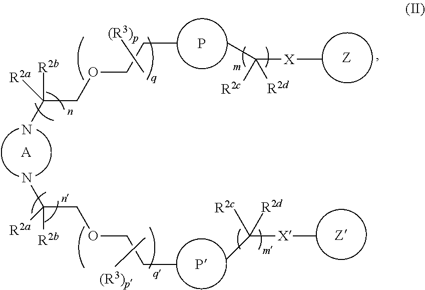

or a pharmaceutically acceptable salt thereof, wherein Rings P and P′ are each independently heterocyclyl or heteroaryl, each of which is optionally substituted with 1-6 R⁴; Rings Z and Z′ are each independently cycloalkyl, heterocyclyl, aryl, or heteroaryl, each of which is optionally substituted with 1-6 R⁵; Ring A is cycloalkyl, heterocyclyl, aryl, or heteroaryl, each of which is optionally substituted with 1-6 R⁶; X and X′ are each independently O, S, N(R^{A}), C₁-C₁₂ alkylene, C₁-C₁₂ alkenylene, C₂-C₁₂ heteroalkylene, or absent; each of R^{2a}, R^{2b}, R^{2c}, and R^{2d} is independently hydrogen, C₁-C₁₂ alkyl, C₂-C₁₂ heteroalkyl, halogen, or OR^{C}; or R^{2a} and R^{2b} or R^{2c} and R^{2d} are taken together to form an oxo; each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B}); each R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B}); each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl; m, m′, n, and n′ are each independently 0, 1, 2, 3, 4, 5, or 6; p and p′ are each independently 0, 1, 2, 3, 4, or 5; and q and q′ are each independently an integer from 0 to 25.

In another aspect, the present disclosure features a compound of Formula (III):

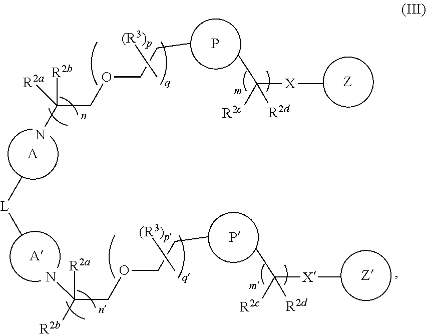

or a pharmaceutically acceptable salt thereof, wherein Rings A and A′ are each independently heterocyclyl or heteroaryl, each of which is optionally substituted with 1-6 R⁶; L is —O—, —C(O)—, —N(R^{A})—, —S(O)ₓ—, C₁-C₁₂ alkylene, C₁-C₁₂ alkenylene, C₂-C₁₂ heteroalkylene, C₁-C₁₂ haloalkylene, or absent, wherein each alkylene, alkenylene, heteroalkylene, and haloalkylene is optionally substituted with 1-6 R⁵; Rings P and P′ are each independently heterocyclyl or heteroaryl, each of which is optionally substituted with 1-6 R⁴; Rings Z and Z′ are each independently cycloalkyl, heterocyclyl, aryl, or heteroaryl, each of which is optionally substituted with 1-6 R⁵; Ring A is cycloalkyl, heterocyclyl, aryl, or heteroaryl, each of which is optionally substituted with 1-6 R⁶; X and X′ are each independently O, S, N(R^{A}), C₁-C₁₂ alkylene, C₁-C₁₂ alkenylene, C₂-C₁₂ heteroalkylene, or absent; each of R^{2a}, R^{2b}, R^{2c}, and R^{2d} is independently hydrogen, C₁-C₁₂ alkyl, C₂-C₁₂ heteroalkyl, halogen, or OR^{C}; or R^{2a} and R^{2b} or R^{2c} and R^{2d} are taken together to form an oxo; each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B}); each R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B} each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl; m, m′, n, and n′ are each independently 0, 1, 2, 3, 4, 5, or 6; p and p′ are each independently 0, 1, 2, 3, 4, or 5; q and q′ are each independently an integer from 0 to 25; and x is 0, 1, or 2.

In an embodiment, the onset temperature of the compound of Formula (I), (II), (III), or a pharmaceutically acceptable salt thereof is greater than about 150° C., e.g., greater than about 175° C., 200° C., 225° C., 250° C., 275° C., 300° C., or 325° C. In an embodiment, the onset temperature of a compound of Formula (I) is greater than about 250° C. In an embodiment, the onset temperature of a compound of Formula (II) is greater than about 250° C. In an embodiment, the onset temperature of a compound of Formula (III) is greater than about 250° C. In an embodiment, the maximum exothermal output of the compound of Formula (I), (II), (III), or a pharmaceutically acceptable salt thereof is lower than about 750 J/g, e.g., lower than about 700 J/g, 650 J/g, 600 J/g, 550 J/g, 500 J/g, or 450 J/g. In an embodiment, the maximum exothermal output is lower than about 300 J/g. In an embodiment, the compound of Formula (I), (II), (III), or a pharmaceutically acceptable salt thereof is a liquid.

In another aspect, the present disclosure features a container comprising greater than about 5 grams of a compound of Formula (I), (II), (III), or a pharmaceutically acceptable salt thereof (e.g., greater than 10 grams, 50 grams, 100 grams, 500 grams, 1 kilogram, 5 kilograms, 10 kilograms, 20 kilograms, or more). In an embodiment, the container is a vial. In an embodiment, the container comprises one or more of glass, metal, and plastic. In an embodiment, the container comprises a lid. In an embodiment, the container comprises a seal (e.g., an airtight seal).

In another aspect, the present disclosure features a preparation comprising greater than about 5 grams of a compound of Formula (I), (II), (III), or a pharmaceutically acceptable salt thereof (e.g., greater than 10 grams, 50 grams, 100 grams, 500 grams, 1 kilogram, 5 kilograms, 10 kilograms, 20 kilograms, or more).

In another aspect, the present disclosure features a method of preparing a compound of Formula (I), (II), (III), or a pharmaceutically acceptable salt thereof. In an embodiment, the method comprises reacting an alkyne compound (e.g., a compound of Formula (V) described herein) with an azide compound (e.g., a compound of Formula (VI) described herein), thereby preparing a compound of Formula (I), (II), or (III). In an embodiment, the reaction occurs in the presence of a catalyst (e.g., a copper catalyst).

The details of one or more embodiments of the invention are set forth herein. Other features, objects, and advantages of the invention will be apparent from the Detailed Description, the Figures, the Examples, and the Claims.

### DETAILED DESCRIPTION

The disclosure provides compounds, e.g., compounds of Formula (I), (II), and (III), as well as related compositions, preparations, and methods of use thereof. The compounds described herein represent useful intermediates in the preparation of certain afibrotic compounds.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“About”, when used herein to modify a numerically defined parameter (e.g., a physical description of a compound as described herein, such as onset temperature), means that the parameter may vary by as much as 15% above or below the stated numerical value for that parameter. For example, a compound defined as having an onset temperature of about 150° C. may have an onset temperature of 127.5° C. to 172.5° C. In some embodiments, the term “about’ means that the parameter may vary by as much as 10% or 5% above or below the stated numerical value for that parameter.

“Acquire” or “acquiring”, as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., analyzing a compound in a differential scanning calorimeter.

“Afibrotic”, as used herein, refers to a compound or material that mitigates the foreign body response (FBR). For example, the amount of FBR in a biological tissue that is induced by implant into that tissue of a device (e.g., hydrogel capsule) comprising an afibrotic compound (e.g., a hydrogel capsule comprising a polymer covalently modified with a compound listed in Tables 1 or 2) is lower than the FBR induced by implantation of an afibrotic-null reference device, i.e., a device that lacks any afibrotic compound, but is of substantially the same composition (e.g., same cell type(s)) and structure (e.g., size, shape, no. of compartments). In an embodiment, the degree of the FBR is assessed by the immunological response in the tissue containing the implanted device (e.g., hydrogel capsule), which may include, for example, protein adsorption, macrophages, multinucleated foreign body giant cells, fibroblasts, and angiogenesis, using assays known in the art, e.g., as described in WO 2017/075630, or using one or more of the assays/methods described Vegas, A., et al., Nature Biotechnol, (e.g., subcutaneous cathepsin measurement of implanted capsules, Masson's trichrome (MT), hematoxylin or eosin staining of tissue sections, quantification of collagen density, cellular staining and confocal microscopy for macrophages (CD68 or F4/80), myofibroblasts (alpha-muscle actin, SMA) or general cellular deposition, quantification of 79 RNA sequences of known inflammation factors and immune cell markers, or FACS analysis for macrophage and neutrophil cells on retrieved devices (e.g., capsules) after 14 days in the intraperitoneal space of a suitable test subject, e.g., an immunocompetent mouse). In an embodiment, the FBR is assessed by measuring the levels in the tissue containing the implant of one or more biomarkers of immune response, e.g., cathepsin, TNF-α, IL-13, IL-6, G-CSF, GM-CSF, IL-4, CCL2, or CCL4. In some embodiments, the FBR induced by a device of the invention (e.g., a hydrogel capsule comprising an afibrotic compound disposed on its outer surface), is at least about 80%, about 85%, about 90%, about 95%, about 99%, or about 100% lower than the FBR induced by an FBR-null reference device, e.g., a device that is substantially identical to the test or claimed device except for lacking the means for mitigating the FBR (e.g., a hydrogel capsule that does not comprise an afibrotic compound but is otherwise substantially identical to a claimed capsule). In some embodiments, the FBR (e.g., level of a biomarker(s)) is measured after about 30 minutes, about 1 hour, about 6 hours, about 12 hours, about 1 day, about 2 days, about 3 days, about 4 days, about 1 week, about 2 weeks, about 1 month, about 2 months, about 3 months, about 6 months, or longer.

“Maximum exothermal output” as used herein, refers to the sum of all exothermic events for a compound (e.g., the sum of all energy generated for each exothermic event). In an embodiment, the maximum exothermal output comprises the total exothermic output related to the major decomposition event of a compound, as well as all prior and subsequent exothermal events. The maximum exothermal output may be measured by the energy per amount of compound (e.g., joules per gram of compound or joules per mole of compound).

“Onset temperature”, as used herein, refers to the temperature at which a compound undergoes a phase transition (e.g., a melting point). In an embodiment, the onset temperature refers to the temperature at which a compound undergoes a chemical decomposition and/or releases energy. In an embodiment, the onset temperature refers to the temperature at which the compound presents a thermal explosion hazard or a runaway reaction hazard. Onset temperatures are often measured by differential scanning calorimetry (DSC) or differential thermal analysis (DTA). Using a calorimetric method, the onset temperature may relate to the intersection of the leading side tangent of the peak in question with the extrapolated baseline. The onset temperature may be impacted by the material housing the calorimetry vessel, e.g., gold-lined or stainless steel. The onset temperature may also be determined using other methods known in the art.

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith & March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”), 2 to 8 carbon atoms (“C₂-C₅ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents, e.g., from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”), 2 to 8 carbon atoms (“C₂-C₈ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., from 1 to 5 substituents, 1 to 3 substituents, or 1 substituent.

As used herein, the term “heteroalkyl” refers to a non-cyclic stable straight or branched chain, or combinations thereof, including at least one carbon atom and at least one heteroatom selected from the group consisting of O, N, P, Si, and S, and wherein the nitrogen, phosphorous, silicon, or sulfur atoms may optionally be oxidized, and the nitrogen heteroatom may optionally be quaternized. The heteroatom(s) 0, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. An alkylene, alkenylene, alkynylene, or heteroalkylene group may be described as, e.g., a C₁-C₆ alkylene, C₂-C₆ alkenylene, C₂-C₆ alkynylene, or C₁-C₆ heteroalkylene. In the case of heteroalkylene groups, heteroatoms can also occupy either or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively. As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₅), bicyclo[1.1.1]pentanyl (C₅), bicyclo[2.2.2]octanyl (C₅), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5] decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, piperazinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl or thiomorpholinyl-1,1-dioxide. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” as used herein refers to the radical —NR^{C}R^{D}, wherein R^{C} and R^{D} are each independently hydrogen, C₁-C₁₂ alkyl, C₃-C₁₀ cycloalkyl, C₃-C₁₀ heterocyclyl, C₆-C₁₀ aryl, and C₅-C₁₀ heteroaryl. In some embodiments, amino refers to NH₂.

As used herein, “cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “hydroxy” refers to the radical —OH.

Alkyl, alkenyl, alkynyl, heteroalkyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present invention contemplates any and all such combinations to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds of Formula (I), (II), or (III), described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, e.g., Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than about 75% by weight, more than about 80% by weight, more than about 85% by weight, more than about 90% by weight, more than about 91% by weight, more than about 92% by weight, more than about 93% by weight, more than about 94% by weight, more than about 95% by weight, more than about 96% by weight, more than about 97% by weight, more than about 98% by weight, more than about 99% by weight, more than about 99.5% by weight, or more than about 99.9% by weight, of the enantiomer. In certain embodiments, the weights are based upon total weight of all enantiomers or stereoisomers of the compound.

Compounds of Formula (I), (II), or (III) described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The term “pharmaceutically acceptable salt” is meant to include salts of the active compounds that are prepared with relatively nontoxic acids or bases, depending on the particular substituents found on the compounds described herein. When compounds used in the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al., J. Pharm. Sci. 66: 1-19 (1977)). Certain specific compounds used in the present disclosure contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

In addition to salt forms, the disclosure may employ compounds of Formula (I), (II), or (III) in a prodrug form. Prodrugs are those compounds that readily undergo chemical changes under physiological conditions to provide the compounds useful in the present invention. Additionally, prodrugs can be converted to useful compounds of Formula (I), (II), or (III) by chemical or biochemical methods in an ex vivo environment.

Certain compounds of Formula (I), (II), or (III) described herein can exist in unsolvated forms as well as solvated forms, including hydrated forms. In general, the solvated forms are equivalent to unsolvated forms and are encompassed within the scope of the present invention. Certain compounds of Formula (I), (II), or (III) described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, dimethylsulfoxide (DMSO), tetrahydrofuran (THF), diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological effect of a compound of interest.

### Compounds of Formula (I), (II), and (III)

The present invention features a compound of Formula (I):

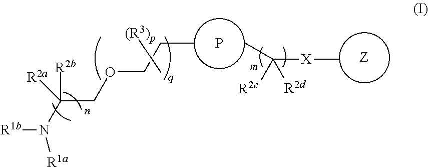

or a pharmaceutical y acceptable salt thereof, wherein Ring P is heterocyclyl or heteroaryl, each of which is optionally substituted with 1-6 R⁴; Ring Z is cycloalkyl, heterocyclyl, aryl, or heteroaryl, each of which is optionally substituted with 1-6 R⁵; X is O, S, N(R^{A}), C₁-C₁₂ alkylene, C₁-C₁₂ alkenylene, C₂-C₁₂ heteroalkylene, or absent; R^{1a} is hydrogen, C₁-C₁₂ alkyl, C₂-C₁₂alkenyl, C₂-C₁₂ alkynyl, C₂-C₁₂ heteroalkyl, C₁-C₁₂ haloalkyl, or an amine-protecting group; R^{1b} is an amine-protecting group; or R^{1a} and R^{1b} may be taken together to form a cycloalkyl, heterocyclyl, aryl, or heteroaryl ring, each of which is optionally substituted with 1-6 R⁶; each of R^{2a}, R^{2b}, R^{2c}, and R^{2d} is independently hydrogen, C₁-C₁₂ alkyl, C₂-C₁₂ heteroalkyl, halogen, or OR^{C}; or R^{2a} and R^{2b} or R^{2c} and R^{2d} are taken together to form an oxo; each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B}); each R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B} each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl; m and n are each independently 0, 1, 2, 3, 4, 5, or 6; p is 0, 1, 2, 3, 4, or 5; and q is an integer from 0 to 25.

In some embodiments, Ring P is heteroaryl optionally substituted with 1-6 R⁴. In some embodiments, Ring P is a monocyclic ring or a bicyclic ring. In an embodiment, Ring P is a monocyclic ring. In an embodiment, Ring P is a 5-membered heteroaryl or 6-membered heteroaryl. In an embodiment, Ring P is a 5-membered heteroaryl. In an embodiment, Ring P is a 6-membered heteroaryl.

In some embodiments, Ring P comprises 1, 2, 3, 4, or 5 heteroatoms. In an embodiment, Ring P comprises 1, 2, or 3 heteroatoms. In an embodiment, Ring P comprises 1 heteroatom. In an embodiment, Ring P comprises 2 heteroatoms. In an embodiment, Ring P comprises 3 heteroatoms. In an embodiment, the heteroatom is a nitrogen atom, oxygen atom, sulfur atom, phosphorus atom, boron atom, or silicon atom.

In some embodiments, P is a monocyclic, nitrogen-containing heteroaryl. In some embodiments, P is a 5-membered nitrogen-containing heteroaryl. In some embodiments, P is tetrazolyl, imidazolyl, pyrazolyl, or triazolyl, pyrrolyl, oxazolyl, or thiazolyl. In some embodiments, P is tetrazolyl, imidazolyl, pyrazolyl, or triazolyl, or pyrrolyl. In some embodiments, P is imidazolyl. In some embodiments, P is triazolyl (e.g., 1,2,3-triazolyl or 1,2,4-triazolyl). In some embodiments, P is 1,2,3-triazolyl. In some embodiments, P is

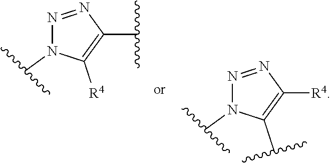

In an embodiment, R⁴ is hydrogen, C₁-C₁₂ alkyl, or halogen.

In some embodiments, Ring Z is a 3-membered ring, 4-membered ring, 5-membered ring, 6-membered ring, 7-membered ring, or 8-membered ring. In an embodiment, Ring Z is a 3-membered ring. In an embodiment, Ring Z is a 4-membered ring. In an embodiment, Ring Z is a 5-membered ring. In an embodiment, Ring Z is a 6-membered ring. In an embodiment, Ring Z is a 7-membered ring. In an embodiment, Ring Z is an 8-membered ring.

In some embodiments, Ring Z comprises at least one heteroatom (e.g., at least two heteroatoms, three heteroatoms, four heteroatoms). The heteroatom may be a nitrogen atom, oxygen atom, sulfur atom, phosphorus atom, boron atom, or silicon atom, and may be further substituted with a substituent, e.g., as described herein.

In an embodiment, Ring Z is a monocyclic ring or a bicyclic ring. In an embodiment, Ring Z is a heterocyclyl optionally substituted with 1-6 R⁴. In an embodiment, Ring Z is a 3-membered, 4-membered, 5-membered, 6-membered, 7-membered, or 8-membered heterocyclyl optionally substituted with 1-6 R⁴. In an embodiment, Ring Z comprises a nitrogen atom, oxygen atom, or sulfur atom. In an embodiment, Ring Z is an oxygen-containing heterocyclyl. In some embodiments, Ring Z is a 6-membered oxygen-containing heterocyclyl. In an embodiment, Ring Z is tetrahydropyranyl. In an embodiment, Ring Z is as follows:

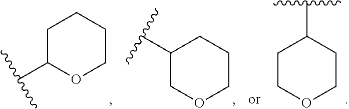

In an embodiment, Ring Z is a 4-membered oxygen-containing heterocyclyl. In an embodiment, Ring Z is

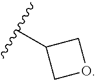 [SMILES: *C1COC1]

In an embodiment, Ring Z is a sulfur-containing heterocyclyl. In an embodiment, Ring Z is a nitrogen-containing heterocyclyl. In an embodiment, Z is a 6-membered nitrogen-containing heterocyclyl. In an embodiment, Ring Z is a 6-membered heterocyclyl containing a nitrogen atom and a sulfur atom. In an embodiment, Ring Z is thiomorpholinyl-1,1-dioxidyl. In an embodiment, Ring, Z is

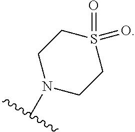 [SMILES: *N1CCS(=O)(=O)CC1]

In an embodiment, Ring Z is

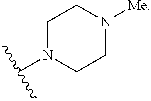 [SMILES: *N1CCN(C)CC1]

In an embodiment, X is absent, O, S, N(R^{A}), C₁-C₁₂ alkylene, or C₂-C₁₂ heteroalkylene.

In an embodiment, X is O. In an embodiment, X is absent.

In an embodiment, each of R^{2a}, R^{2b}, R^{2c}, and R^{2d} is independently hydrogen, C₁-C₁₂ alkyl, C₂-C₁₂ heteroalkyl, or OR^{C}. In an embodiment, each of R^{2a} and R^{2b} is independently hydrogen.

In an embodiment, each of R^{2c} and R^{2d} is independently hydrogen. In an embodiment, each of R^{2a} and R^{2b} is independently hydrogen.

In an embodiment, R^{1a} is hydrogen, C₁-C₁₂ alkyl, C₂-C₁₂ alkenyl, C₂-C₁₂ heteroalkyl, C₁-C₁₂ haloalkyl, or an amine-protecting group. In an embodiment, R^{1a} is hydrogen, C₁-C₁₂ alkyl, C₂-C₁₂ heteroalkyl, or an amine-protecting group. In an embodiment, R^{1a} is hydrogen or an amine-protecting group. In an embodiment, R^{1a} is hydrogen. In an embodiment, R^{1a} is an amine-protecting group. In an embodiment, R^{1a} is an acid-labile amine-protecting group, a base-labile protecting group, an ultraviolet light-labile protecting group, or an amine-protecting group removed by hydrogenation.

In an embodiment, R^{1b} is an acid-labile amine-protecting group, a base-labile protecting group, an ultraviolet light-labile protecting group, or an amine-protecting group removed by hydrogenation. In an embodiment, R^{1b} is an amine-protecting group selected from 9-fluorenylmethoxycarbonyl (Fmoc), benzyl (Bn), benzoyl (Bz), allyloxycarbonyl (Alloc), 2-(4-nitropheylsulfonyl)ethoxycarbonyl (Nsc), 1,1-dioxobenzo[b]thiophene-2-ylmethyloxycarbonyl (Bsmoc), 1,1-dioxonaphtho[1,2-b]thiophene (Nsmoc), 1-(4,4-dimethyl-2,6-dioxocyclohex-1-ylidene)-3-methylbutyl (ivDde), 2,2,2-trichloroethyloxycarbonyl (Troc), 2-[phenyl(methyl)sulfonio]ethyloxycarbonyl tetrafluoroborate (Pms), tert-butyloxycarbonyl (Boc), p-methoxybenzyl (PMB), carbobenzyloxy (Cbz), acetyl (Ac), tosyl (Ts), trityl (Trt), 2-(4-biphenyl)-isopropoxycarbonyl (Bpoc), 2,2,5,5-tetramethyl-1,2,5-azadisilolidine, 1,3-dithiolan-2-imine, nitrobenzenesulfonyl (Nosyl), and pyromellitic diimide. In an embodiment, R^{1b} is 9-fluorenylmethoxycarbonyl (Fmoc). In an embodiment, R^{1b} is benzyl (Bn). In an embodiment, R^{1b} is benzoyl (Bz). In an embodiment, R^{1b} is allyloxycarbonyl (Alloc). In an embodiment, R^{1b} is 2-(4-nitropheylsulfonyl)ethoxycarbonyl (Nsc). In an embodiment, R^{1b} is 1,1-dioxobenzo[b]thiophene-2-ylmethyloxycarbonyl (Bsmoc). In an embodiment, R^{1b} is 1,1-dioxonaphtho[1,2-b]thiophene (Nsmoc). In an embodiment, R^{1b} is 1-(4,4-dimethyl-2,6-dioxocyclohex-1-ylidene)-3-methylbutyl (ivDde). In an embodiment, R^{1b} is 2,2,2-trichloroethyloxycarbonyl (Troc). In an embodiment, R^{1b} is 2-[phenyl(methyl)sulfonio]-ethyloxycarbonyl tetrafluoroborate (Pms). In an embodiment, R^{1b} is tert-butyloxycarbonyl (Boc). In an embodiment, R^{1b} is p-methoxybenzyl (PMB). In an embodiment, R^{1b} is carbobenzyloxy (Cbz). In an embodiment, R^{1b} is acetyl (Ac). In an embodiment, R^{1b} is tosyl (Ts). In an embodiment, R^{1b} is trityl (Trt). In an embodiment, R^{1b} is 2-(4-biphenyl)-isopropoxycarbonyl (Bpoc). In an embodiment, R^{1b} is 2,2,5,5-tetramethyl-1,2,5-azadisilolidine. In an embodiment, R^{1b} is 1,3-dithiolan-2-imine, nitrobenzenesulfonyl (Nosyl). In an embodiment, R^{1b} is and pyromellitic diimide.

In an embodiment, R^{1a} is hydrogen and R^{1b} is an amine protecting group selected from 9-fluorenylmethoxycarbonyl (Fmoc), benzyl (Bn), benzoyl (Bz), allyloxycarbonyl (Alloc), 2-(4-nitropheylsulfonyl)ethoxycarbonyl (Nsc), 1,1-dioxobenzo[b]thiophene-2-ylmethyloxycarbonyl (Bsmoc), 1,1-dioxonaphtho[1,2-b]thiophene (Nsmoc), 1-(4,4-dimethyl-2,6-dioxocyclohex-1-ylidene)-3-methylbutyl (ivDde), 2,2,2-trichloroethyloxycarbonyl (Troc), 2-[phenyl(methyl)sulfonio]ethyloxycarbonyl tetrafluoroborate (Pms), tert-butyloxycarbonyl (Boc), p-methoxybenzyl (PMB), carbobenzyloxy (Cbz), acetyl (Ac), tosyl (Ts), trityl (Trt), 2-(4-biphenyl) isopropoxycarbonyl (Bpoc), 2,2,5,5-tetramethyl-1,2,5-azadisilolidine, 1,3-dithiolan-2-imine, nitrobenzenesulfonyl (Nosyl), and pyromellitic diimide. In an embodiment, R^{1a} is hydrogen and R^{1b} is tert-butyloxycarbonyl (Boc).

In an embodiment, m is 0, 1, 2, 3, or 4. In an embodiment, m is 0. In an embodiment, m is 1. In an embodiment, m is 2. In an embodiment, m is 3. In an embodiment, m is 4.

In an embodiment, n is 0, 1, 2, 3, or 4. In an embodiment, n is 0. In an embodiment, n is 1. In an embodiment, n is 2. In an embodiment, n is 3. In an embodiment, n is 4.

In an embodiment, each of m and n is independently 0 or 1. In an embodiment, each of m and n is independent 1. In an embodiment, one of m and n is independently 0 and the other of m and n is independently 1.

In an embodiment, q is 1, 2, 3, 4, 5, or 6. In an embodiment, q is 2, 3, or 4. In an embodiment, q is 2. In an embodiment, q is 3. In an embodiment, q is 4.

In an embodiment, p is 0, 1, or 2. In an embodiment, p is 0. In an embodiment, p is 1. In an embodiment, p is 2.

In some embodiments, the compound of Formula (I) is a compound of Formula (I-a):

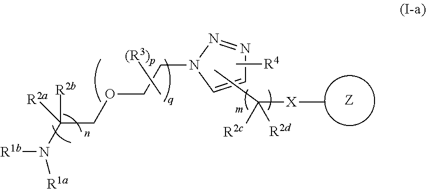

or a pharmaceutically acceptable salt thereof, wherein Ring Z is cycloalkyl, heterocyclyl, aryl, or heteroaryl, each of which is optionally substituted with 1-6 R⁵; X is O, S, N(R^{A}), C₁-C₁₂ alkylene, C₁-C₁₂ alkenylene, C₂-C₁₂ heteroalkylene, or absent; R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group; R^{1b} is an amine-protecting group; or R^{1a} and R^{1b} may be taken together to form a cycloalkyl, heterocyclyl, aryl, or heteroaryl ring, each of which is optionally substituted with 1-6 R⁶; each of R^{2a}, R^{2b}, R^{2c}, and R^{2d} is independently hydrogen, C₁-C₁₂ alkyl, C₂-C₁₂ heteroalkyl, halogen, or OR^{C}; or R^{2a} and R^{2b} or R^{2c} and R^{2d} are taken together to form an oxo; each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B}); R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B}); each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl; m and n are each independently 0, 1, 2, 3, 4, 5, or 6; p is 0, 1, 2, 3, 4, or 5; and q is an integer from 0 to 25.

In some embodiments, the compound of Formula (I) is a compound of Formula (I-b):

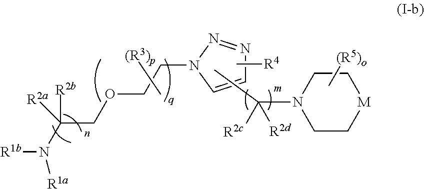

or a pharmaceutically acceptable salt thereof, wherein M is C(R′)(R″), N(R′), or S(O)ₓ; each of R′ and R″ is independently hydrogen, C₁-C₁₂ alkyl, C₂-C₁₂ heteroalkyl, C₁-C₁₂ haloalkyl or halogen; or each of R′ and R″ is taken together to form an oxo; R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group; R^{1b} is an amine-protecting group; or R^{1a} and R^{1b} may be taken together to form a cycloalkyl, heterocyclyl, aryl, or heteroaryl ring, each of which is optionally substituted with 1-6 R⁶; each of R^{2a}, R^{2b}, R^{2c}, and R^{2d} is independently hydrogen, C₁-C₁₂ alkyl, C₂-C₁₂ heteroalkyl, or halogen; or R^{2a} and R^{2b} or R^{2c} and R^{2d} are taken together to form an oxo; each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B}); R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B}); each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl; m and n are each independently 0, 1, 2, 3, 4, 5, or 6; o and p are each independently 0, 1, 2, 3, 4, or 5; q is an integer from 0 to 25; and x is 0, 1, or 2.

In some embodiments, the compound of Formula (I) is a compound of Formula (I-d):

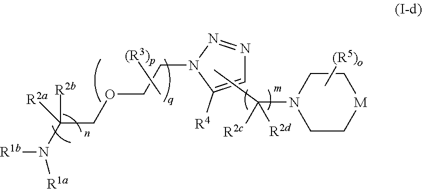

or a pharmaceutically acceptable salt thereof, wherein M is C(R′)(R″), N(R′), or S(O)ₓ; each of R′ and R″ is independently hydrogen, C₁-C₁₂ alkyl, C₂-C₁₂ heteroalkyl, C₁-C₁₂ haloalkyl or halogen; or each of R′ and R″ is taken together to form an oxo; R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group; R^{1b} is an amine-protecting group; or R^{1a} and R^{1b} may be taken together to form a cycloalkyl, heterocyclyl, aryl, or heteroaryl ring, each of which is optionally substituted with 1-6 R⁶; each of R^{2a}, R^{2b}, R^{2c}, and R^{2d} is independently hydrogen, C₁-C₁₂ alkyl, C₂-C₁₂ heteroalkyl, or halogen; or R^{2a} and R^{2b} or R^{2c} and R^{2d} are taken together to form an oxo; each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B}); R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B}); each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl; m and n are each independently 0, 1, 2, 3, 4, 5, or 6; o and p are each independently 0, 1, 2, 3, 4, or 5; q is an integer from 0 to 25; and x is 0, 1, or 2.

In some embodiments, the compound is a compound of Formula (I-e):

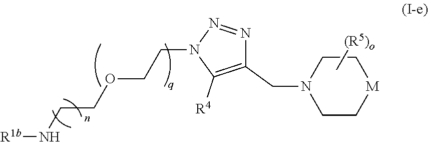

or a pharmaceutically acceptable salt thereof, wherein M is C(R′)(R″), N(R′), or S(O)ₓ; each of R′ and R″ is independently hydrogen, C₁-C₁₂ alkyl, C₂-C₁₂ heteroalkyl, C₁-C₁₂ haloalkyl or halogen; or each of R′ and R″ is taken together to form an oxo; R^{1b} is an amine-protecting group; R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; each R⁵ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B}); each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl; n is 0, 1, 2, 3, 4, 5, or 6; o is 0, 1, 2, 3, 4, or 5; q is an integer from 0 to 25; and x is 0, 1, or 2.

In an embodiment, the compound is a compound of Formula (I-f):

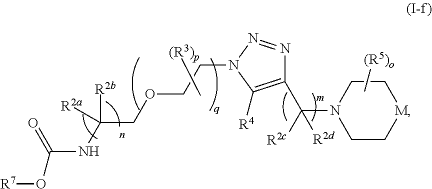

or a pharmaceutically acceptable salt thereof, wherein M is C(R′)(R″), N(R′), or S(O)ₓ; each of R′ and R″ is independently hydrogen, C₁-C₁₂ alkyl, C₂-C₁₂ heteroalkyl, C₁-C₁₂ haloalkyl, or halogen; or each of R′ and R″ is taken together to form an oxo; each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B}); R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B}); R⁷ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, cycloalkyl, heterocyclyl, aryl, or heteroaryl, wherein alkyl, alkenyl, alkynyl, heteroalkyl, haloalkyl, cycloalkyl, heterocyclyl, aryl, and heteroaryl is optionally substituted with 1-6 R⁶; each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl; m and n are each independently 0, 1, 2, 3, 4, 5, or 6; o and p are each independently 0, 1, 2, 3, 4, or 5; q is an integer from 0 to 25; and x is 0, 1, or 2.

In an embodiment, the compound is a compound of Formula (I-g):

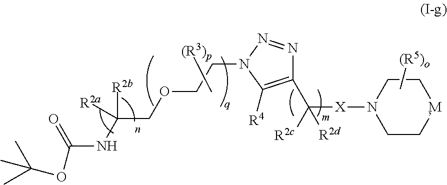

or a pharmaceutically acceptable salt thereof, wherein M is C(R′)(R″), N(R′), or S(O)ₓ; each of R′ and R″ is independently hydrogen, C₁-C₁₂ alkyl, C₂-C₁₂ heteroalkyl, C₁-C₁₂ haloalkyl, or halogen; or each of R′ and R″ is taken together to form an oxo; X is O, S, N(R^{A}), C₁-C₁₂ alkylene, C₁-C₁₂ alkenylene, C₂-C₁₂ heteroalkylene, or absent; each of R^{2a}, R^{2b}, R^{2c}, and R^{2d} is independently hydrogen, C₁-C₁₂ alkyl, C₂-C₁₂ heteroalkyl, or halogen; or each of R^{2a}, R^{2b}, R^{2c}, and R^{2d} is independently hydrogen, C₁-C₁₂alkyl, C₂-C₁₂ heteroalkyl, or halogen; or R^{2a} and R^{2b} or R^{2c} and R^{2d} are taken together to form an oxo; each of R³ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B}); R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B}); each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl; m and n are each independently 0, 1, 2, 3, 4, 5, or 6; o and p are each independently 0, 1, 2, 3, 4, or 5; q is an integer from 0 to 25; and x is 0, 1, or 2.

In an embodiment, the compound is a compound of Formula (I-h):

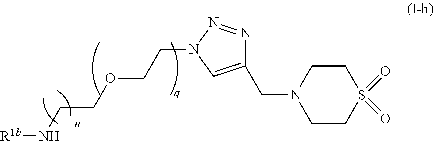

or a pharmaceutically acceptable salt thereof, wherein R^{1b} is an amine-protecting group; n is 0, 1, 2, 3, 4, 5, or 6; and q is an integer from 0 to 25.

In another aspect, the present disclosure features a compound of Formula (II):

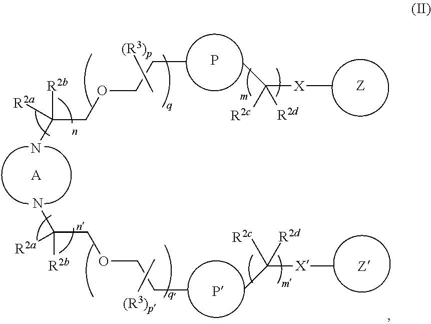

or a pharmaceutically acceptable salt thereof, wherein Rings P and P′ are each independently heterocyclyl or heteroaryl, each of which is optionally substituted with 1-6 R⁴; Rings Z and Z′ are each independently cycloalkyl, heterocyclyl, aryl, or heteroaryl, each of which is optionally substituted with 1-6 R⁵; Ring A is cycloalkyl, heterocyclyl, aryl, or heteroaryl, each of which is optionally substituted with 1-6 R⁶; X and X′ are each independently O, S, N(R^{A}), C₁-C₁₂ alkylene, C₁-C₁₂ alkenylene, C₂-C₁₂ heteroalkylene, or absent; each of R^{2a}, R^{2b}, R^{2c}, and R^{2d} is independently hydrogen, C₁-C₁₂ alkyl, C₂-C₁₂ heteroalkyl, halogen, or OR^{C}; or R^{2a} and R^{2b} or R^{2c} and R^{2d} are taken together to form an oxo; each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B}); each R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B}); each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl; m, m′, n, and n′ are each independently 0, 1, 2, 3, 4, 5, or 6; p and p′ are each independently 0, 1, 2, 3, 4, or 5; and q and q′ are each independently an integer from 0 to 25.

In some embodiments, Rings P and P′ are heteroaryl optionally substituted with 1-6 R⁴. In some embodiments, Rings Ps and P′ are a monocyclic ring or a bicyclic ring. In an embodiment, Rings P and P′ are a monocyclic ring. In an embodiment, Rings P and P′ are a 5-membered heteroaryl or 6-membered heteroaryl. In an embodiment, Rings P and P′ are a 5-membered heteroaryl. In an embodiment, Rings P and P′ are a 6-membered heteroaryl.

In some embodiments, Rings P and P′ comprise 1, 2, 3, 4, or 5 heteroatoms. In an embodiment, Rings P and P′ comprise 1, 2, or 3 heteroatoms. In an embodiment, Rings P and P′ comprise 1 heteroatom. In an embodiment, Rings P and P′ comprise 2 heteroatoms. In an embodiment, Rings P and P′ comprise 3 heteroatoms. In an embodiment, the heteroatom is a nitrogen atom, oxygen atom, sulfur atom, phosphorus atom, boron atom, or silicon atom.

In some embodiments, Rings P and P′ are each a monocyclic, nitrogen-containing heteroaryl. In some embodiments, Rings P and P′ are each a 5-membered nitrogen-containing heteroaryl. In some embodiments, Rings P and P′ are each tetrazolyl, imidazolyl, pyrazolyl, or triazolyl, pyrrolyl, oxazolyl, or thiazolyl. In some embodiments, Rings P and P′ are each tetrazolyl, imidazolyl, pyrazolyl, or triazolyl, or pyrrolyl. In some embodiments, Rings P and P′ are each imidazolyl. In some embodiments, Rings P and P′ are each triazolyl (e.g., 1,2,3-triazolyl or 1,2,4-triazolyl). In some embodiments, Ring P is 1,2,3-triazolyl. In some embodiments, Ring P′ is 1,2,3-triazolyl. In some embodiments, Ring P is

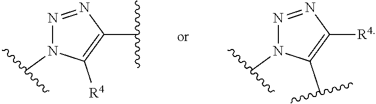

In some embodiments, Ring P′ is

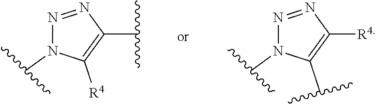

In an embodiment, each R⁴ is hydrogen, C₁-C₁₂ alkyl, or halogen.

In some embodiments, Ring A is a monocyclic, bicyclic, or tricyclic ring. In an embodiment, Ring A is a tricyclic ring. In an embodiment, Ring A is a heterocyclyl or heteroaryl. In an embodiment, Ring A is a tricyclic heterocyclyl. In an embodiment, Ring A comprises a succinimidyl ring or a component thereof. In an embodiment, Ring A tetracyclic ring. In an embodiment, Ring A is a pentacyclic ring. In an embodiment, Ring A is a hexacyclic ring. In an embodiment, Ring A is a heptacyclic ring. In an embodiment, Ring A is an octocyclic ring. In an embodiment, Ring A is

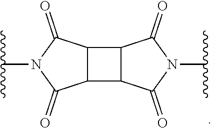 [SMILES: *N1C(=O)C2C(C1=O)C1C(=O)N(*)C(=O)C21]

In some embodiments, Rings Z and Z′ are each a 3-membered ring, 4-membered ring, 5-membered ring, 6-membered ring, 7-membered ring, or 8-membered ring. In an embodiment, Rings Z and Z′ are each a 3-membered ring. In an embodiment, Rings Z and Z′ are each a 4-membered ring. In an embodiment, Rings Z and Z′ are each a 5-membered ring. In an embodiment, Rings Z and Z′ are each a 6-membered ring. In an embodiment, Ring Z is a 7-membered ring. In an embodiment, Rings Z and Z′ are each an 8-membered ring.

In some embodiments, Rings Z and Z′ each comprise at least one heteroatom (e.g., at least two heteroatoms, three heteroatoms, four heteroatoms). The heteroatom may be a nitrogen atom, oxygen atom, sulfur atom, phosphorus atom, boron atom, or silicon atom, and may be further substituted with a substituent, e.g., as described herein.

In an embodiment, Rings Z and Z′ are each a monocyclic ring or a bicyclic ring. In an embodiment, Ring Z is a heterocyclyl optionally substituted with 1-6 R⁴. In an embodiment, Ring Z′ is a heterocyclyl optionally substituted with 1-6 R⁴. In an embodiment, Rings Z and Z′ are each a 3-membered, 4-membered, 5-membered, 6-membered, 7-membered, or 8-membered heterocyclyl optionally substituted with 1-6 R⁴. In an embodiment, Rings Z and Z′ each comprise a nitrogen atom, oxygen atom, or sulfur atom. In an embodiment, Rings Z and Z′ are each an oxygen-containing heterocyclyl. In some embodiments, Ring Z is a 6-membered oxygen-containing heterocyclyl. In some embodiments, Ring Z′ is a 6-membered oxygen-containing heterocyclyl. In an embodiment, Rings Z and Z′ are tetrahydropyranyl. In an embodiment, Rings Z and Z′ are each selected from

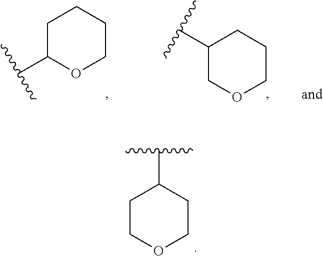 [SMILES: *C1CCCCO1 ; *C1CCCOC1 ; *C1CCOCC1]

In an embodiment, Rings Z and Z′ are each a 4-membered oxygen-containing heterocyclyl. In an embodiment, Ring Z is

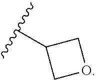 [SMILES: *C1COC1]

In an embodiment, each of Rings Z and Z′ is a sulfur-containing heterocyclyl. In an embodiment, each of Rings Z and Z′ is a nitrogen-containing heterocyclyl. In an embodiment, each of Rings Z and Z′ is a 6-membered nitrogen-containing heterocyclyl. In an embodiment, each of Rings Z and Z′ is a 6-membered heterocyclyl containing a nitrogen atom and a sulfur atom. In an embodiment, each of Rings Z and Z′ is thiomorpholinyl-1,1-dioxidyl. In an embodiment, each of Rings Z and Z′ is

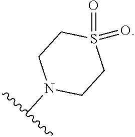 [SMILES: *N1CCS(=O)(=O)CC1]

In an embodiment, each of Rings Z and Z′ is

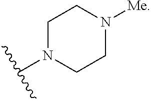 [SMILES: *N1CCN(C)CC1]

In some embodiments, each of X and X′ is absent, O, S, N(R^{A}), C₁-C₁₂ alkylene, or C₂-C₁₂ heteroalkylene. In an embodiment, X is O. In an embodiment, X is absent. In an embodiment, X′ is O. In an embodiment, X′ is absent. In an embodiment, each of X and X′ is independently absent. In an embodiment, each of X and X′ is independently O.

In some embodiments, each of R^{2a}, R^{2b}, R^{2C}, and R^{2d} is independently hydrogen, C₁-C₁₂ alkyl, C₂-C₁₂ heteroalkyl, or OR^{C}. In an embodiment, each of R^{2a} and R^{2b} is independently hydrogen. In an embodiment, each of R^{2C} and R^{2d} is independently hydrogen. In an embodiment, each of R^{2a} and R^{2b} is independently hydrogen.

In some embodiments, each of m and m′ is 1 or 2. In an embodiment, m is 1 or 2. In an embodiment, m′ is 1 or 2. In an embodiment, m is 1. In an embodiment, m′ is 1.

In some embodiments, each of n and n′ is 1 or 2. In an embodiment, n is 1 or 2. In an embodiment, n′ is 1 or 2. In an embodiment, n is 1. In an embodiment, n′ is 1.

In some embodiments, each of q and q′ is 1, 2, 3, 4, 5, or 6. In an embodiment, each of q and q′ is 2, 3, or 4. In an embodiment, q is 2, 3, or 4. In an embodiment, q′ is 2, 3, or 4.

In some embodiments, each of p and p′ is 0. In an embodiment, p is 0. In an embodiment, p′ is 0.

In another aspect, the present disclosure features a compound of Formula (III):

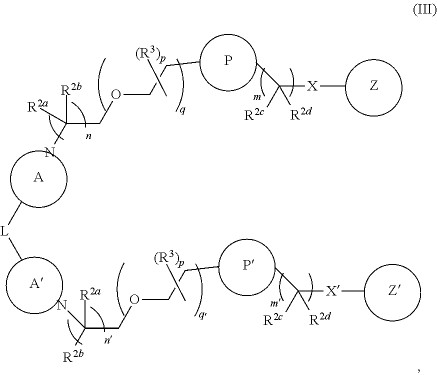

or a pharmaceutically acceptable salt thereof, wherein Rings A and A′ are each independently heterocyclyl or heteroaryl, each of which is optionally substituted with 1-6 R⁶; L is —O—, —C(O)—, —N(R^{A})—, —S(O)ₓ—, C₁-C₁₂ alkylene, C₁-C₁₂ alkenylene, C₂-C₁₂ heteroalkylene, C₁-C₁₂ haloalkylene, or absent, wherein each alkylene, alkenylene, heteroalkylene, and haloalkylene is optionally substituted with 1-6 R⁵; Rings P and P′ are each independently heterocyclyl or heteroaryl, each of which is optionally substituted with 1-6 R⁴; Rings Z and Z′ are each independently cycloalkyl, heterocyclyl, aryl, or heteroaryl, each of which is optionally substituted with 1-6 R⁵; X and X′ are each independently O, S, N(R^{A}), C₁-C₁₂ alkylene, C₁-C₁₂ alkenylene, C₂-C₁₂ heteroalkylene, or absent; each of R^{2a}, R^{2b}, R^{2c}, and R^{2d} is independently hydrogen, C₁-C₁₂ alkyl, C₂-C₁₂ heteroalkyl, halogen, or OR^{C}; or R^{2a} and R^{2b} or R^{2c} and R^{2d} are taken together to form an oxo; each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B}); each R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B}); each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl; m, m′, n, and n′ are each independently 0, 1, 2, 3, 4, 5, or 6; p and p′ are each independently 0, 1, 2, 3, 4, or 5; q and q′ are each independently an integer from 0 to 25; and x is 0, 1, or 2.

In some embodiments, Rings A and A′ are each independently monocyclic, bicyclic, or tricyclic rings. In some embodiments, Rings A and A′ optionally substituted with 1-6 R⁶. In some embodiments, Rings A and A′ are each heterocyclyl (e.g., nitrogen-containing heterocyclyl). In some embodiments, Rings A and A′ are each phthalimidyl.

In some embodiments, L is absent, —O—, —C(O)—, —S(O)ₓ—, or C₁-C₁₂ alkylene optionally substituted with one or more R⁵. In some embodiments, L is absent. In some embodiments, L is —O—. In some embodiments, L is —C(O)—. In some embodiments, L is —S(O)ₓ— (e.g., —SO₂—). In some embodiments, L is C₁-C₁₂ alkylene (e.g., C(CH₂CF₃)₂).

In some embodiments, Rings P and P′ are heteroaryl optionally substituted with 1-6 R⁴. In some embodiments, Rings Ps and P′ are a monocyclic ring or a bicyclic ring. In an embodiment, Rings P and P′ are a monocyclic ring. In an embodiment, Rings P and P′ are a 5-membered heteroaryl or 6-membered heteroaryl. In an embodiment, Rings P and P′ are a 5-membered heteroaryl. In an embodiment, Rings P and P′ are a 6-membered heteroaryl.

In some embodiments, Rings P and P′ comprise 1, 2, 3, 4, or 5 heteroatoms. In an embodiment, Rings P and P′ comprise 1, 2, or 3 heteroatoms. In an embodiment, Rings P and P′ comprise 1 heteroatom. In an embodiment, Rings P and P′ comprise 2 heteroatoms. In an embodiment, Rings P and P′ comprise 3 heteroatoms. In an embodiment, the heteroatom is a nitrogen atom, oxygen atom, sulfur atom, phosphorus atom, boron atom, or silicon atom.

In some embodiments, Rings P and P′ are each a monocyclic, nitrogen-containing heteroaryl. In some embodiments, Rings P and P′ are each a 5-membered nitrogen-containing heteroaryl. In some embodiments, Rings P and P′ are each tetrazolyl, imidazolyl, pyrazolyl, or triazolyl, pyrrolyl, oxazolyl, or thiazolyl. In some embodiments, Rings P and P′ are each tetrazolyl, imidazolyl, pyrazolyl, or triazolyl, or pyrrolyl. In some embodiments, Rings P and P′ are each imidazolyl. In some embodiments, Rings P and P′ are each triazolyl (e.g., 1,2,3-triazolyl or 1,2,4-triazolyl). In some embodiments, Ring P is 1,2,3-triazolyl. In some embodiments, Ring P′ is 1,2,3-triazolyl. In some embodiments, Ring P is

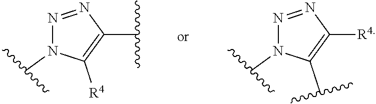

In some embodiments, Ring P′ is

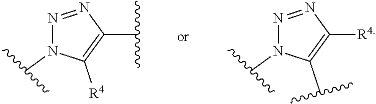

In an embodiment, each R⁴ is hydrogen, C₁-C₁₂ alkyl, or halogen.

In some embodiments, Rings Z and Z′ are each a 3-membered ring, 4-membered ring, 5-membered ring, 6-membered ring, 7-membered ring, or 8-membered ring. In an embodiment, Rings Z and Z′ are each a 3-membered ring. In an embodiment, Rings Z and Z′ are each a 4-membered ring. In an embodiment, Rings Z and Z′ are each a 5-membered ring. In an embodiment, Rings Z and Z′ are each a 6-membered ring. In an embodiment, Ring Z is a 7-membered ring. In an embodiment, Rings Z and Z′ are each an 8-membered ring.

In some embodiments, Rings Z and Z′ each comprise at least one heteroatom (e.g., at least two heteroatoms, three heteroatoms, four heteroatoms). The heteroatom may be a nitrogen atom, oxygen atom, sulfur atom, phosphorus atom, boron atom, or silicon atom, and may be further substituted with a substituent, e.g., as described herein.

In an embodiment, Rings Z and Z′ are each a monocyclic ring or a bicyclic ring. In an embodiment, Ring Z is a heterocyclyl optionally substituted with 1-6 R⁴. In an embodiment, Ring Z′ is a heterocyclyl optionally substituted with 1-6 R⁴. In an embodiment, Rings Z and Z′ are each a 3-membered, 4-membered, 5-membered, 6-membered, 7-membered, or 8-membered heterocyclyl optionally substituted with 1-6 R⁴. In an embodiment, Rings Z and Z′ each comprise a nitrogen atom, oxygen atom, or sulfur atom. In an embodiment, Rings Z and Z′ are each an oxygen-containing heterocyclyl. In some embodiments, Ring Z is a 6-membered oxygen-containing heterocyclyl. In some embodiments, Ring Z′ is a 6-membered oxygen-containing heterocyclyl. In an embodiment, Rings Z and Z′ are tetrahydropyranyl. In an embodiment, Rings Z and Z′ are each selected from

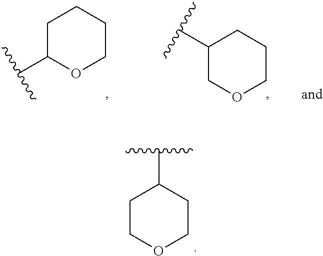 [SMILES: *C1CCCCO1 ; *C1CCCOC1 ; *C1CCOCC1]

In an embodiment, Rings Z and Z′ are each a 4-membered oxygen-containing heterocyclyl. In an embodiment, Ring Z is

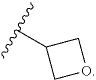 [SMILES: *C1COC1]

In an embodiment, each of Rings Z and Z′ is a sulfur-containing heterocyclyl. In an embodiment, each of Rings Z and Z′ is a nitrogen-containing heterocyclyl. In an embodiment, each of Rings Z and Z′ is a 6-membered nitrogen-containing heterocyclyl. In an embodiment, each of Rings Z and Z′ is a 6-membered heterocyclyl containing a nitrogen atom and a sulfur atom. In an embodiment, each of Rings Z and Z′ is thiomorpholinyl-1,1-dioxidyl. In an embodiment, each of Rings Z and Z′ is

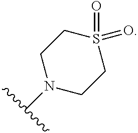 [SMILES: *N1CCS(=O)(=O)CC1]

In an embodiment, each of Rings Z and Z′ is

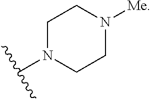 [SMILES: *N1CCN(C)CC1]

In some embodiments, each of X and X′ is absent, O, S, N(R^{A}), C₁-C₁₂ alkylene, or C₂-C₁₂ heteroalkylene. In an embodiment, X is O. In an embodiment, X is absent. In an embodiment, X′ is O. In an embodiment, X′ is absent. In an embodiment, each of X and X′ is independently absent. In an embodiment, each of X and X′ is independently O.

In some embodiments, each of R^{2a}, R^{2b}, R^{2c}, and R^{2d} is independently hydrogen, C₁-C₁₂ alkyl, C₂-C₁₂ heteroalkyl, or OR^{C}. In an embodiment, each of R^{2a} and R^{2b} is independently hydrogen. In an embodiment, each of R^{2c} and R^{2d} is independently hydrogen. In an embodiment, each of R^{2a} and R^{2b} is independently hydrogen.

In some embodiments, each of m and m′ is 1 or 2. In an embodiment, m is 1 or 2. In an embodiment, m′ is 1 or 2. In an embodiment, m is 1. In an embodiment, m′ is 1.

In some embodiments, each of n and n′ is 1 or 2. In an embodiment, n is 1 or 2. In an embodiment, n′ is 1 or 2. In an embodiment, n is 1. In an embodiment, n′ is 1.

In some embodiments, each of q and q′ is 1, 2, 3, 4, 5, or 6. In an embodiment, each of q and q′ is 2, 3, or 4. In an embodiment, q is 2, 3, or 4. In an embodiment, q′ is 2, 3, or 4.

In some embodiments, each of p and p′ is 0. In an embodiment, p is 0. In an embodiment, p′ is 0.

TABLE 1
Exemplary compounds of Formula (I), (II), and (III)

| Compound No. | Structure |
|---|---|
| 100 | 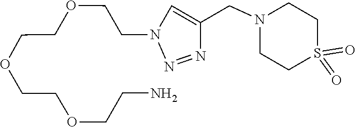 |
| 101 | 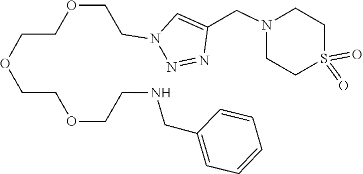 [SMILES: O=S1(=O)CCN(Cc2cn(CCOCCOCCOCCNCc3ccccc3)nn2)CC1] |
| 102 | 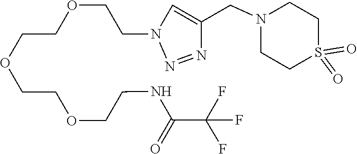 |
| 103 | 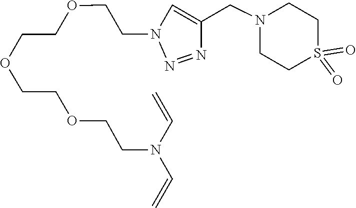 |
| 104 | 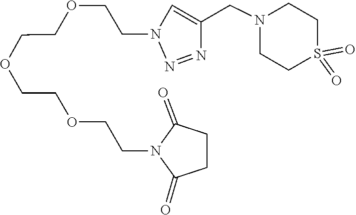 |
| 105 | 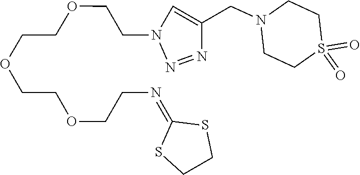 |
| 106 | 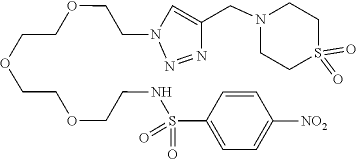 |
| 107 | 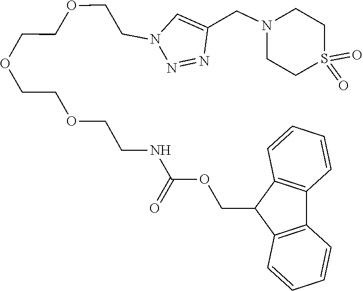 [SMILES: O=C(NCCOCCOCCOCCn1cc(CN2CCS(=O)(=O)CC2)nn1)OCC1c2ccccc2-c2ccccc21] |
| 108 | 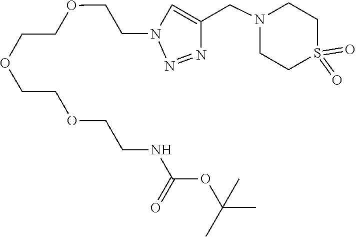 |
| 109 | 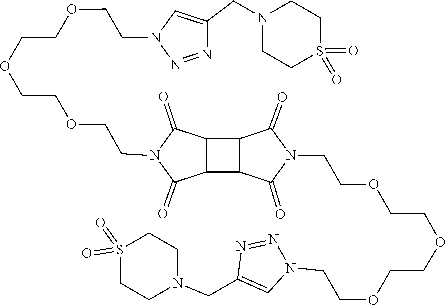 [SMILES: O=C1C2C(C(=O)N1CCOCCOCCOCCn1cc(CN3CCS(=O)(=O)CC3)nn1)C1C(=O)N(CCOCCOCCOCCn3cc(CN4CCS(=O)(=O)CC4)nn3)C(=O)C21] |
| 110 | 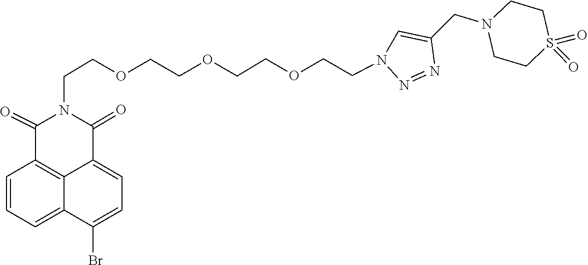 [SMILES: O=C1c2cccc3c(Br)ccc(c23)C(=O)N1CCOCCOCCOCCn1cc(CN2CCS(=O)(=O)CC2)nn1] |
| 111 | 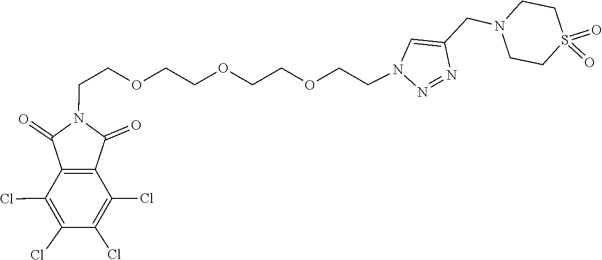 [SMILES: O=C1c2c(Cl)c(Cl)c(Cl)c(Cl)c2C(=O)N1CCOCCOCCOCCn1cc(CN2CCS(=O)(=O)CC2)nn1] |
| 112 | 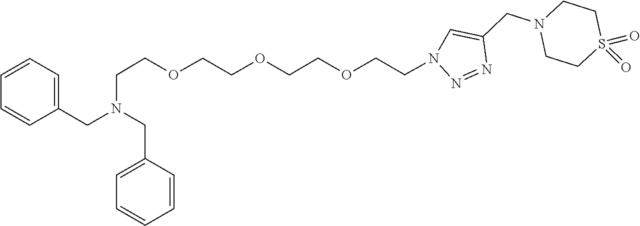 [SMILES: O=S1(=O)CCN(Cc2cn(CCOCCOCCOCCN(Cc3ccccc3)Cc3ccccc3)nn2)CC1] |
| 113 | 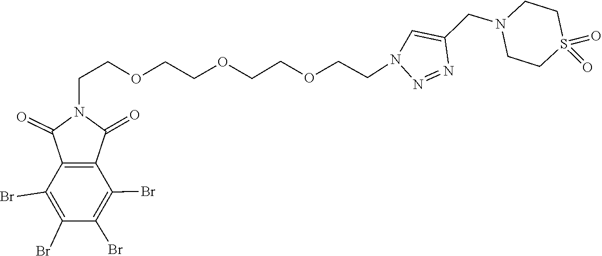 [SMILES: O=C1c2c(Br)c(Br)c(Br)c(Br)c2C(=O)N1CCOCCOCCOCCn1cc(CN2CCS(=O)(=O)CC2)nn1] |
| 114 | 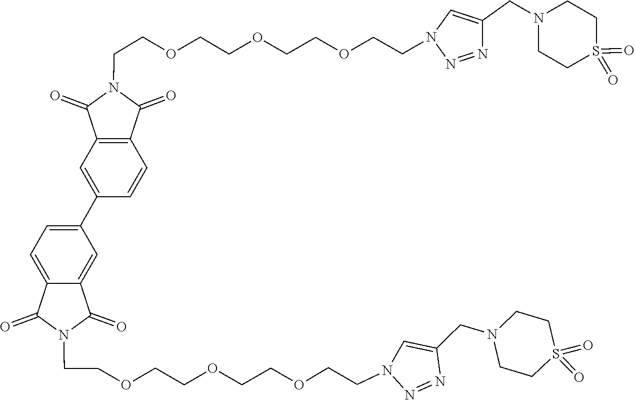 [SMILES: O=C1c2ccc(-c3ccc4c(c3)C(=O)N(CCOCCOCCOCCn3cc(CN5CCS(=O)(=O)CC5)nn3)C4=O)cc2C(=O)N1CCOCCOCCOCCn1cc(CN2CCS(=O)(=O)CC2)nn1] |
| 115 | 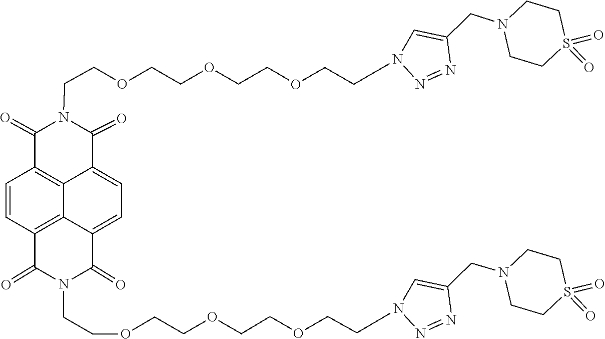 [SMILES: O=C1c2ccc3c4c(ccc(c24)C(=O)N1CCOCCOCCOCCn1cc(CN2CCS(=O)(=O)CC2)nn1)C(=O)N(CCOCCOCCOCCn1cc(CN2CCS(=O)(=O)CC2)nn1)C3=O] |
| 116 | 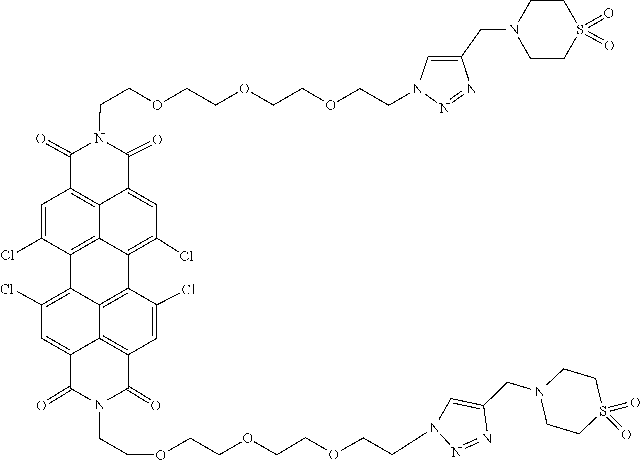 [SMILES: O=C1c2cc(Cl)c3c4c(Cl)cc5c6c(cc(Cl)c(c7c(Cl)cc(c2c37)C(=O)N1CCOCCOCCOCCn1cc(CN2CCS(=O)(=O)CC2)nn1)c64)C(=O)N(CCOCCOCCOCCn1cc(CN2CCS(=O)(=O)CC2)nn1)C5=O] |
| 117 | 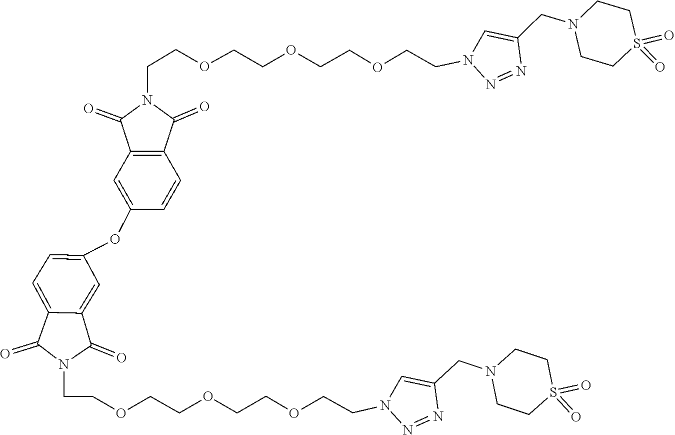 [SMILES: O=C1c2ccc(Oc3ccc4c(c3)C(=O)N(CCOCCOCCOCCn3cc(CN5CCS(=O)(=O)CC5)nn3)C4=O)cc2C(=O)N1CCOCCOCCOCCn1cc(CN2CCS(=O)(=O)CC2)nn1] |
| 118 | 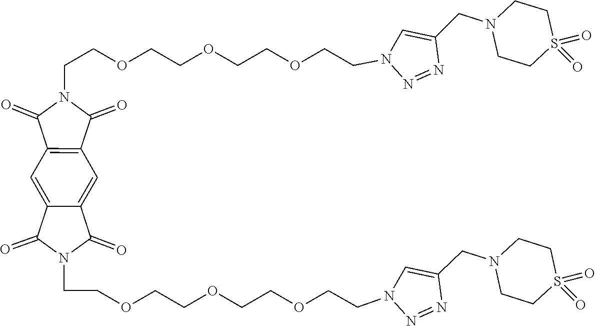 [SMILES: O=c1c2cc3c(=O)n(CCOCCOCCOCCn4cc(CN5CCS(=O)(=O)CC5)nn4)c(=O)c3cc2c(=O)n1CCOCCOCCOCCn1cc(CN2CCS(=O)(=O)CC2)nn1] |
| 119 | 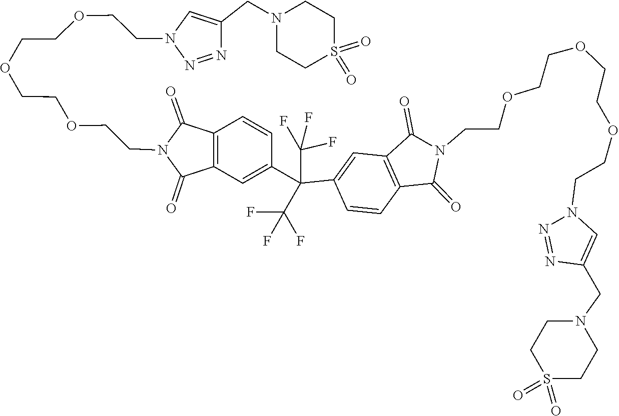 [SMILES: O=C1c2ccc(C(c3ccc4c(c3)C(=O)N(CCOCCOCCOCCn3cc(CN5CCS(=O)(=O)CC5)nn3)C4=O)(C(F)(F)F)C(F)(F)F)cc2C(=O)N1CCOCCOCCOCCn1cc(CN2CCS(=O)(=O)CC2)nn1] |
| 120 | 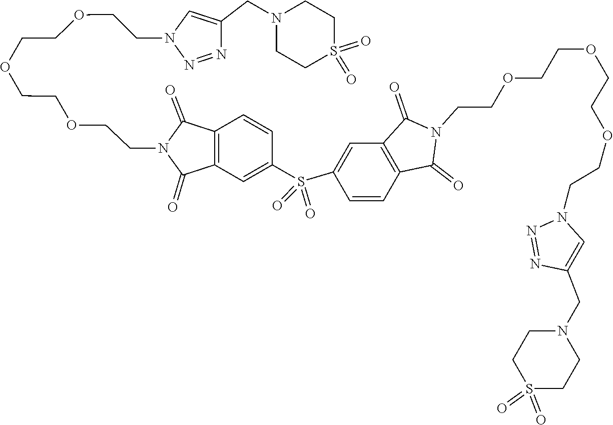 [SMILES: O=C1c2ccc(S(=O)(=O)c3ccc4c(c3)C(=O)N(CCOCCOCCOCCn3cc(CN5CCS(=O)(=O)CC5)nn3)C4=O)cc2C(=O)N1CCOCCOCCOCCn1cc(CN2CCS(=O)(=O)CC2)nn1] |
| 121 | 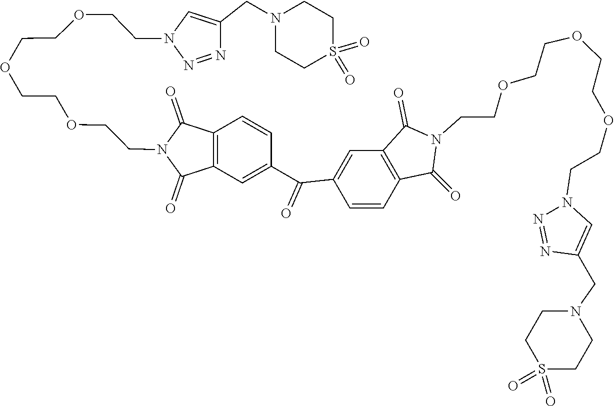 [SMILES: O=C(c1ccc2c(c1)C(=O)N(CCOCCOCCOCCn1cc(CN3CCS(=O)(=O)CC3)nn1)C2=O)c1ccc2c(c1)C(=O)N(CCOCCOCCOCCn1cc(CN3CCS(=O)(=O)CC3)nn1)C2=O] |
| 122 | 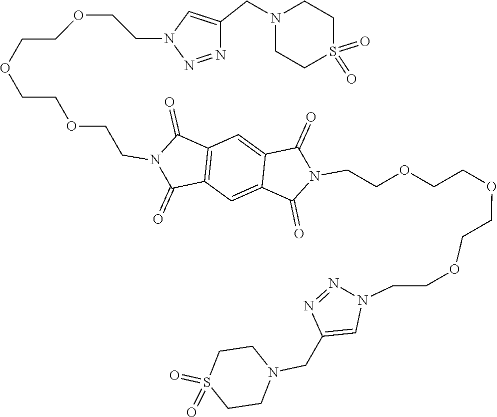 [SMILES: O=c1c2cc3c(=O)n(CCOCCOCCOCCn4cc(CN5CCS(=O)(=O)CC5)nn4)c(=O)c3cc2c(=O)n1CCOCCOCCOCCn1cc(CN2CCS(=O)(=O)CC2)nn1] |

Certain nitrogen-containing compounds, such as azide compounds, may be reactive and pose an explosion hazard and/or shock hazard when handling. In particular, these compounds may be prone to violent decomposition from an external energy source, including light, heat, friction, or pressure, and require special care when handling to minimize risk. Further, these nitrogen-containing compounds may have toxic properties. As such, new methods and intermediates have been developed to circumvent the use of these compounds to reduce the associated handling risks. In an embodiment, the compounds of Formula (I) (e.g., compounds of Formula (I-a), (I-b), (I-c), (I-d), (I-e), (I-f), (I-g), and (I-h)) and Formula (II) or pharmaceutically acceptable salts thereof have improved properties over their azide counterparts. For example, the compounds of Formula (I) (e.g., compounds of Formula (I-a), (I-b), (I-c), (I-d), (I-e), (I-f), (I-g), and (I-h)) and Formula (II) or pharmaceutically acceptable salts thereof may have a greater onset temperature or lower maximum exothermal output than their azide counterparts.

In some embodiments, the compounds of Formula (I), (I-a), (I-b), (I-c), (I-d), (I-e), (I-f), (I-g), (I-h), and (II), or a pharmaceutically acceptable salt thereof, have an onset temperature greater than about 150° C. In an embodiment, the onset temperature is greater than about 160° C., e.g., greater than about 175° C., 200° C., 225° C., 250° C., 275° C., 300° C., or 325° C. In some embodiments, the onset temperature of the compound is between about 150° C. and 350° C., e.g., between about 200° C. and 350° C. or between about 250° C. and 350° C. In an embodiment, the onset temperature of a compound of Formula (I) or (II) is greater than 200° C. In an embodiment, the onset temperature of a compound of Formula (I) or (II) is greater than 250° C. In an embodiment, the onset temperature of a compound of Formula (I) or (II) is greater than 275° C. In an embodiment, the onset temperature of a compound of Formula (I) or (II) is greater than 300° C.

In some embodiments, the compounds of Formula (I), (I-a), (I-b), (I-c), (I-d), (I-e), (I-f), (I-g), (I-h), (II), (III), or a pharmaceutically acceptable salt thereof, have a maximum exothermal output lower than about 750 J/g, e.g., lower than about 700 J/g, 650 J/g, 600 J/g, 550 J/g, 500 J/g, or 450 J/g. In an embodiment, the maximum exothermal output of the compound is between about 100 J/g and 700 J/g, e.g., 200 J/g and 600 J/g or between 250 J/g and 550 J/g.

In some embodiments, the compounds of Formula (I), (I-a), (I-b), (I-c), (I-d), (I-e), (I-f), (I-g), (I-h), (II), (III), or a pharmaceutically acceptable salt thereof, have an onset temperature greater than about 150° C. In an embodiment, the onset temperature is greater than about 160° C., e.g., greater than about 175° C., 200° C., 225° C., 250° C., 275° C., 300° C., or 325° C. In some embodiments, the onset temperature of the compound is between about 150° C. and 350° C., e.g., between about 200° C. and 350° C. or between about 250° C. and 350° C. In an embodiment, the onset temperature of a compound of Formula (I), (II), or (III) is greater than 200° C. In an embodiment, the onset temperature of a compound of Formula (I), (II), or (III) is greater than 250° C. In an embodiment, the onset temperature of a compound of Formula (I), (II), or (III) is greater than 275° C. In an embodiment, the onset temperature of a compound of Formula (I), (II), or (III) is greater than 300° C.

In some embodiments, the compounds of Formula (I), (I-a), (I-b), (I-c), (I-d), (I-e), (I-f), (I-g), (I-h), (II), (III), or a pharmaceutically acceptable salt thereof, have a maximum exothermal output lower than about 750 J/g, e.g., lower than about 700 J/g, 650 J/g, 600 J/g, 550 J/g, 500 J/g, or 450 J/g. In an embodiment, the maximum exothermal output of the compound is between about 100 J/g and 700 J/g, e.g., 200 J/g and 600 J/g or between 250 J/g and 550 J/g.

The “nitrogen ratio” of a compound (e.g., a compound described herein) may be determined using the equation set forth below:

$$\frac{Carbons + Oxygens + Sulfus + Halogens}{Nitrogens} = Nitrogen Ratio$$

wherein the value of “carbons,” “oxygens,” “sulfurs,” “nitrogens,” and “halogens” refers to the number of each of these types of atoms in a compound. Generally, the nitrogen ratio may provide a reference guide for the volatility of a particular compound (e.g., an azide compound), where a nitrogen ratio of below 4.0 may indicate a potential explosion hazard; see, e.g., Brase & Banert, Organic Azides: Synthesis and Applications (Wiley: Chichester, 2010) and Kolb et al. (2001) Angew Chem Int Ed 40: 2004-2021. In an embodiment, the nitrogen ratio of a compound of Formula (I) or (II) is greater than about 2.0, e.g., 2.25, 2.5, 2.75, 3.0, 3.25, 3.5, 3.75, 4.0, 4.25, 4.5, 4.75, 5.0, 5.25, 5.5, 5.75, or 6. In an embodiment, the nitrogen ratio of a compound of Formula (I) or (II) is greater than 4.0. In an embodiment, the nitrogen ratio of a compound of Formula (I) or (II) is greater than 4.5. In an embodiment, the nitrogen ratio of a compound of Formula (I) or (II) is greater than 5.0. In an embodiment, the nitrogen ratio of a compound of Formula (I) or (II) is greater than 5.5. In an embodiment, the nitrogen ratio of a compound of Formula (I) or (II) is greater than 5.0, and the onset temperature of the compound of Formula (I) or (II) is greater than about 150° C., e.g., greater than about 160° C., 175° C., 200° C., 225° C., 250° C., 275° C., 300° C., or 325° C. In an embodiment, the nitrogen ratio of a compound of Formula (I) or (II) is greater than 5.0 and the maximum exothermal output of the compound of Formula (I) or (II) is lower than about 750 J/g, e.g., lower than about 700 J/g, 650 J/g, 600 J/g, 550 J/g, 500 J/g, or 450 J/g.

In an embodiment, the nitrogen ratio of a compound of Formula (I), (II), or (III) is greater than 4.0. In an embodiment, the nitrogen ratio of a compound of Formula (I), (II), or (III) is greater than 4.5. In an embodiment, the nitrogen ratio of a compound of Formula (I), (II), or (III) is greater than 5.0. In an embodiment, the nitrogen ratio of a compound of Formula (I), (II), or (III) is greater than 5.5. In an embodiment, the nitrogen ratio of a compound of Formula (I), (II), or (III) is greater than 5.0, and the onset temperature of the compound of Formula (I), (II), or (III) is greater than about 150° C., e.g., greater than about 160° C., 175° C., 200° C., 225° C., 250° C., 275° C., 300° C., or 325° C. In an embodiment, the nitrogen ratio of a compound of Formula (I), (II), or (III) is greater than 5.0 and the maximum exothermal output of the compound of Formula (I), (II), or (III) is lower than about 750 J/g, e.g., lower than about 700 J/g, 650 J/g, 600 J/g, 550 J/g, 500 J/g, or 450 J/g.

In another aspect, the present disclosure features a container comprising greater than about 5 grams of a compound of Formula (I), (II), or (III) or a pharmaceutically acceptable salt thereof (e.g., greater than 10 grams, 50 grams, 100 grams, 500 grams, 1 kilogram, 5 kilograms, 10 kilograms, 20 kilograms, or more). The container may comprise between about 10 grams and 100 kilograms of a compound of Formula (I), (II), or (III), e.g., 10 grams and 10 kilograms, 10 grams and 1 kilogram, 10 grams and 500 grams, 10 grams and 250 grams, 10 grams and 100 grams or 10 grams and 50 grams of a compound of Formula (I), (II), or (III). Alternatively, the container may comprise between about 100 grams and 100 kilograms of a compound of Formula (I), (II), or (III), e.g., 100 grams and 50 kilograms, 100 grams and 25 kilograms, 100 grams and 10 kilograms, 100 grams and 5 kilograms, 100 grams and 1 kilogram, 100 grams and 500 grams, or 100 grams and 150 grams of a compound of Formula (I), (II), or (III). In an embodiment, the container comprises a vial, ampule, bottle, tube, syringe, and/or dispenser package, or other suitable container. In an embodiment, the container is a vial. In an embodiment, the container comprises an inert material, e.g., glass or metal. In an embodiment, the container comprises one or more of glass, metal, and plastic. In an embodiment, the container comprises glass. In an embodiment, the container comprises plastic. In an embodiment, the container comprises a plastic bag (e.g., a polybag), which may be formed from polyethylene, e.g., low density polyethylene. In embodiment, the plastic bag containing the compound is disposed in a drum, e.g., a fibreboard, plastic or metal drum. In an embodiment, the container does not comprise metal. In an embodiment, the container comprises a lid. In an embodiment, the container comprises a seal (e.g., an airtight seal).

In another aspect, the present disclosure features a preparation comprising greater than about 5 grams of a compound of Formula (I), (II), or (III) or a pharmaceutically acceptable salt thereof (e.g., greater than 10 grams, 50 grams, 100 grams, 500 grams, 1 kilogram, 5 kilograms, 10 kilograms, 20 kilograms, or more). The preparation of a compound of Formula (I), (II), or (III) may comprise an additive, reactant, or impurity found in a method of making the compound of Formula (I), (II), or (III), respectively. In an embodiment, the preparation is substantially pure. In an embodiment, the preparation comprises less than about 25%, 20%, 15%, 12.5%, 10%. 9%, 8%, 7%, 6%, 5%, 4%, 3%, 2%, 1%, 0.5% of a second compound (e.g., an intermediate in the method of making the compound of Formula (I), (II), or (III)). In an embodiment, the second compound is an intermediate, side product, or impurity prepared in the synthesis of a compound of Formula (I), (II), or (III)

In another aspect, the present disclosure features compositions of a compound of Formula (I), (II), or (III) or a pharmaceutically acceptable salt thereof which comprise less than about 25%, 20%, 15%, 12.5%, 10%. 9%, 8%, 7%, 6%, 5%, 4%, 3%, 2%1, 1%, 0.75%, 0.5%, 0.25%, 0.1%, or 0.05% (w/w, w/v, v/v, or % by dry weight) of a second compound. In some embodiments, the second compound is present in an amount between 10% and 1% (w/w, w/v, v/v, or % by dry weight) of the composition. In some embodiments, the second compound is present in an amount between 5% and 1% (w/w, w/v, v/v, or % by dry weight) of the composition. In some embodiments, the second compound is present in an amount between 5% and 0.5% (w/w, w/v, v/v, or % by dry weight) of the composition. In some embodiments, the second compound is present in an amount between 2.5% and 0.05% (w/w, w/v, v/v, or % by dry weight) of the composition. In some embodiments, the second compound is present in an amount between 5% and 0.1% (w/w, w/v, v/v, or % by dry weight) of the composition. In some embodiments, the second compound is present in an amount between 2.5% and 0.1% (w/w, w/v, v/v, or % by dry weight) of the composition. In an embodiment, the second compound is an intermediate, side product, or impurity prepared in the synthesis of a compound of Formula (I), (II), or (III).

In some embodiments, the second compound is present in an amount less than 10% (w/w, w/v, v/v, or % by dry weight) of the composition. In some embodiments, the second compound is present in an amount less than 5% (w/w, w/v, v/v, or % by dry weight) of the composition. In some embodiments, the second compound is present in an amount less than 4% (w/w, w/v, v/v, or % by dry weight) of the composition. In some embodiments, the second compound is present in an amount less than 3% (w/w, w/v, v/v, or % by dry weight) of the composition. In some embodiments, the second compound is present in an amount less than 2% (w/w, w/v, v/v, or % by dry weight) of the composition. In some embodiments, the second compound is present in an amount less than 1% (w/w, w/v, v/v, or % by dry weight) of the composition. In some embodiments, the second compound is present in an amount less than 0.9% (w/w, w/v, v/v, or % by dry weight) of the composition. In some embodiments, the second compound is present in an amount less than 0.8% (w/w, w/v, v/v, or % by dry weight) of the composition. In some embodiments, the second compound is present in an amount less than 0.7% (w/w, w/v, v/v, or % by dry weight) of the composition. In some embodiments, the second compound is present in an amount less than 0.6% (w/w, w/v, v/v, or % by dry weight) of the composition. In some embodiments, the second compound is present in an amount less than 0.5% (w/w, w/v, v/v, or % by dry weight) of the composition. In some embodiments, the second compound is present in an amount less than 0.4% (w/w, w/v, v/v, or % by dry weight) of the composition. In some embodiments, the second compound is present in an amount less than 0.3% (w/w, w/v, v/v, or % by dry weight) of the composition. In some embodiments, the second compound is present in an amount less than 0.2% (w/w, w/v, v/v, or % by dry weight) of the composition. In some embodiments, the second compound is present in an amount less than 0.1% (w/w, w/v, v/v, or % by dry weight) of the composition. In some embodiments, the second compound is present in an amount less than 0.05% (w/w, w/v, v/v, or % by dry weight) of the composition.

In another aspect, the present disclosure features compositions of a compound of Formula (I), (II), or (III) or a pharmaceutically acceptable salt thereof which comprise a second compound at an amount greater than about 0.05%, 0.1%, 0.2%, 0.3%, 0.4%, 0.5%, 0.6%, 0.7%, 0.8%, 0.9%, 1%, 1.5%, 2%, 2.5%. 3%, 4%, or 5% (w/w, w/v, v/v, or % by dry weight) of the composition. In an embodiment, the second compound is present at amount greater than about 0.05% (w/w, w/v, v/v, or % by dry weight) of the composition. In an embodiment, the second compound is present at amount greater than about 0.1% (w/w, w/v, v/v, or % by dry weight) of the composition. In an embodiment, the second compound is present at amount greater than about 0.2% (w/w, w/v, v/v, or % by dry weight) of the composition. In an embodiment, the second compound is present at amount greater than about 0.25% (w/w, w/v, v/v, or % by dry weight) of the composition. In an embodiment, the second compound is present at amount greater than about 0.3% (w/w, w/v, v/v, or % by dry weight) of the composition. In an embodiment, the second compound is present at amount greater than about 0.4% (w/w, w/v, v/v, or % by dry weight) of the composition. In an embodiment, the second compound is present at amount greater than about 0.5% (w/w, w/v, v/v, or % by dry weight) of the composition. In an embodiment, the second compound is present at amount greater than about 0.75% (w/w, w/v, v/v, or % by dry weight) of the composition.

In another aspect, the present disclosure features compositions of a compound of Formula (I), (II), or (III) or a pharmaceutically acceptable salt thereof which comprise a second compound (e.g., a compound of Formula (IV)) at a ratio of the compound of Formula (I), (II), or (III) to the second compound greater than 90:10, e.g., 95:5, 97:2.5, 98:2, 99:1, 99.5:0.5, 99.9:0.1 (w/w, w/v, v/v, or molar ratio).

In an embodiment, the second compound is selected from a compound in Table 2 below.

TABLE 2
Exemplary compounds of Formula (IV)

| Compound No. | Structure |
|---|---|
| 200 | 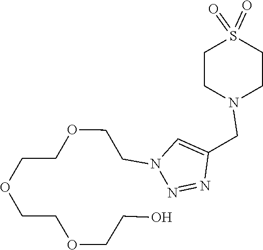 [SMILES: O=S1(=O)CCN(Cc2cn(CCOCCOCCOCCO)nn2)CC1] |
| 201 | 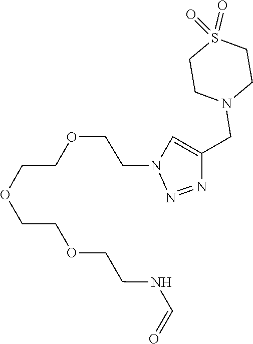 |
| 202 | 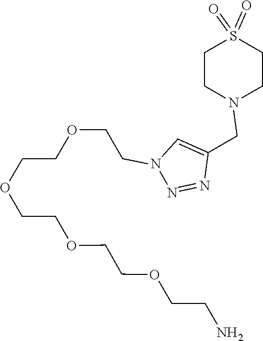 [SMILES: NCCOCCOCCOCCOCCn1cc(CN2CCS(=O)(=O)CC2)nn1] |
| 203 | 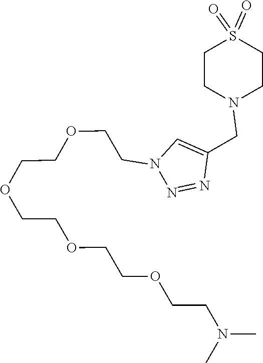 [SMILES: CN(C)CCOCCOCCOCCOCCn1cc(CN2CCS(=O)(=O)CC2)nn1] |
| 204 | 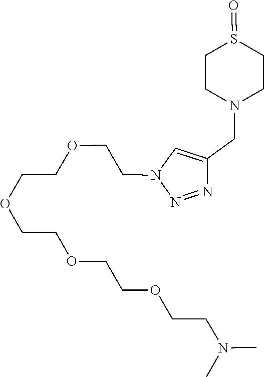 [SMILES: CN(C)CCOCCOCCOCCOCCn1cc(CN2CCS(=O)CC2)nn1] |
| 205 | 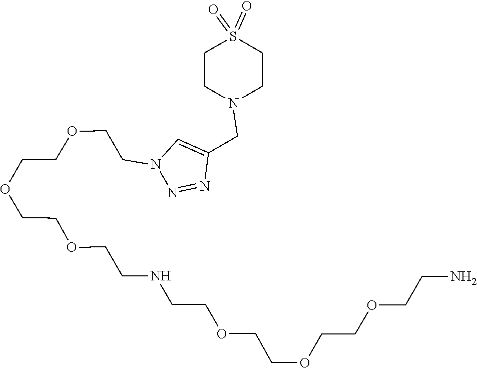 [SMILES: NCCOCCOCCOCCNCCOCCOCCOCCn1cc(CN2CCS(=O)(=O)CC2)nn1] |
| 206 | 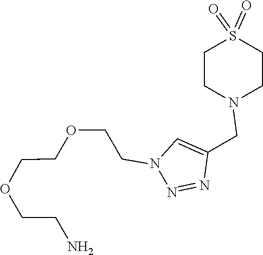 [SMILES: NCCOCCOCCn1cc(CN2CCS(=O)(=O)CC2)nn1] |
| 207 | 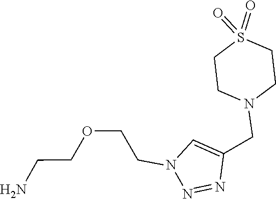 [SMILES: NCCOCCn1cc(CN2CCS(=O)(=O)CC2)nn1] |

In some embodiments, the second compound (e.g., the compound of Formula (IV)) is Compound 200. In some embodiments, the composition of Formula (I), (II), or (III) comprises an amount of Compound 200 greater than about 0.05% (e.g., greater than about 0.1%, 0.15%, 0.2%, 0.25%, 0.3%, 0.4%, 0.5%, 0.6%, 0.7%, 0.8%, 0.9%, or 1%) of the total composition.

In some embodiments, the second compound (e.g., the compound of Formula (IV)) is Compound 201. In some embodiments, the composition of Formula (I), (II), or (III) comprises an amount of Compound 201 greater than about 0.05% (e.g., greater than about 0.1%, 0.15%, 0.2%, 0.25%, 0.3%, 0.4%, 0.5%, 0.6%, 0.7%, 0.8%, 0.9%, or 1%) of the total composition.

In some embodiments, the second compound (e.g., the compound of Formula (IV)) is Compound 202. In some embodiments, the composition of Formula (I), (II), or (III) comprises an amount of Compound 202 greater than about 0.05% (e.g., greater than about 0.1%, 0.15%, 0.2%, 0.25%, 0.3%, 0.4%, 0.5%, 0.6%, 0.7%, 0.8%, 0.9%, or 1%) of the total composition.

In some embodiments, the second compound (e.g., the compound of Formula (IV)) is Compound 203. In some embodiments, the composition of Formula (I), (II), or (III) comprises an amount of Compound 200 greater than about 0.05% (e.g., greater than about 0.1%, 0.15%, 0.2%, 0.25%, 0.3%, 0.4%, 0.5%, 0.6%, 0.7%, 0.8%, 0.9%, or 1%) of the total composition.

In some embodiments, the second compound (e.g., the compound of Formula (IV)) is Compound 204. In some embodiments, the composition of Formula (I), (II), or (III) comprises an amount of Compound 204 greater than about 0.05% (e.g., greater than about 0.1%, 0.15%, 0.2%, 0.25%, 0.3%, 0.4%, 0.5%, 0.6%, 0.7%, 0.8%, 0.9%, or 1%) of the total composition.

In some embodiments, the second compound (e.g., the compound of Formula (IV)) is Compound 205. In some embodiments, the composition of Formula (I), (II), or (III) comprises an amount of Compound 205 greater than about 0.05% (e.g., greater than about 0.1%, 0.15%, 0.2%, 0.25%, 0.3%, 0.4%, 0.5%, 0.6%, 0.7%, 0.8%, 0.9%, or 1%) of the total composition.

In some embodiments, the second compound (e.g., the compound of Formula (IV)) is Compound 206. In some embodiments, the composition of Formula (I), (II), or (III) comprises an amount of Compound 206 greater than about 0.05% (e.g., greater than about 0.1%, 0.15%, 0.2%, 0.25%, 0.3%, 0.4%, 0.5%, 0.6%, 0.7%, 0.8%, 0.9%, or 1%) of the total composition.

In some embodiments, the second compound (e.g., the compound of Formula (IV)) is Compound 207. In some embodiments, the composition of Formula (I), (II), or (III) comprises an amount of Compound 207 greater than about 0.05% (e.g., greater than about 0.1%, 0.15%, 0.2%, 0.25%, 0.3%, 0.4%, 0.5%, 0.6%, 0.7%, 0.8%, 0.9%, or 1%) of the total composition.

### ENUMERATED EMBODIMENTS

1. A compound of Formula (I):

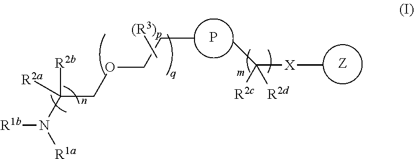

or a pharmaceutically acceptable salt thereof, wherein:
   Ring P is heterocyclyl or heteroaryl, each of which is optionally substituted with 1-6 R⁴;
   Ring Z is cycloalkyl, heterocyclyl, aryl, or heteroaryl, each of which is optionally substituted with 1-6 R⁵;
   X is O, S, N(R^{A}), C₁-C₁₂ alkylene, C₁-C₁₂ alkenylene, C₂-C₁₂ heteroalkylene, or absent;
   R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group;
   R^{1b} is an amine-protecting group; or
   R^{1a} and R^{1b} may be taken together to form a cycloalkyl, heterocyclyl, aryl, or heteroaryl ring, each of which is optionally substituted with 1-6 R⁶;
   each of R^{2a}, R^{2b}, R^{2c}, and R^{2d} is independently hydrogen, C₁-C₁₂ alkyl, C₂-C₁₂ heteroalkyl, halogen, or OR^{C}; or
   R^{2a} and R^{2b} or R^{2c} and R^{2d} are taken together to form an oxo;
   each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   each R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
   m and n are each independently 0, 1, 2, 3, 4, 5, or 6;
   p is 0, 1, 2, 3, 4, or 5; and
   q is an integer from 0 to 25.
2. The compound of embodiment 1, wherein Ring P is a heteroaryl.
3. The compound of any one of embodiments 1-2, wherein Ring P is a nitrogen-containing heteroaryl.
4. The compound of any one of embodiments 1-3, wherein Ring P is a five-membered or six-membered nitrogen heteroaryl.
5. The compound of any one of embodiments 1-4, wherein Ring P is triazolyl (e.g., 1,2,3-triazolyl or 1,2,4-triazolyl).
6. The compound of embodiments 5, wherein Ring P is selected from

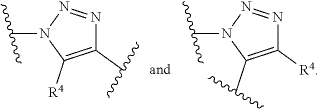

7. The compound of any one of embodiments 1-6, wherein Ring Z is a four-membered ring, a five-membered ring, or a six-membered ring (e.g., a six-membered ring).
8. The compound of any one of embodiments 1-7, wherein Ring Z is heterocyclyl or heteroaryl (e.g., Ring Z comprises at least one heteroatom (e.g., a nitrogen, oxygen, or sulfur)).
9. The compound of any one of embodiments 1-8, wherein Ring Z is a nitrogen-containing six-membered ring.
10. The compound of any one of embodiments 1-8, wherein Ring Z is an oxygen-containing six-membered ring.
11. The compound of any one of embodiments 1-10, wherein R^{1a} is hydrogen.
12. The compound of any one of embodiments 1-11, wherein R^{1b} comprises a C₁-C₁₂ alkylene, C₂-C₁₂ alkenylene, C₂-C₁₂ heteroalkylene, C₁-C₁₂ haloalkylene, cycloalkyl, heterocyclyl, aryl, or heteroaryl.
13. The compound of any one of embodiments 1-12, wherein R^{1b} comprises an acid-labile amine-protecting group or a base-labile amine-protecting group.
14. The compound of any one of embodiments 1-13, wherein R^{1b} is selected from 9-fluorenylmethoxycarbonyl (Fmoc), benzyl (Bn), benzoyl (Bz), allyloxycarbonyl (Alloc), 2-(4-nitropheylsulfonyl)ethoxycarbonyl (Nsc), 1,1-dioxobenzo[b]thiophene-2-ylmethyloxycarbonyl (Bsmoc), 1,1-dioxonaphtho[1,2-b]thiophene (Nsmoc), 1-(4,4-dimethyl-2,6-dioxocyclohex-1-ylidene)-3-methylbutyl (ivDde), 2,2,2-trichloroethyloxycarbonyl (Troc), 2-[phenyl(methyl)sulfonio]ethyloxycarbonyl tetrafluoroborate (Pms), tert-butyloxycarbonyl (Boc), p-methoxybenzyl (PMB), carbobenzyloxy (Cbz), acetyl (Ac), trifluoroacetyl, tosyl (Ts), trityl (Trt), 2-(4-biphenyl)-isopropoxycarbonyl (Bpoc), allyl (Al), 2,2,5,5-tetramethyl-1,2,5-azadisilolidine, 1,3-dithiolan-2-imine, nitrobenzenesulfonyl (Nosyl), succinimidyl, and pyromellitic diimide.
15. The compound of any one of embodiments 1-14, wherein R^{1b} is selected from tert-butyloxycarbonyl (Boc), 9-fluorenylmethoxycarbonyl (Fmoc), benzyl (Bn), allyl (Al), nitrobenzenesulfonyl (Nosyl), dithiolan-2-imine, and trifluoroacetyl.
16. The compound of any one of embodiments 1-15, wherein R^{1b} is tert-butyloxycarbonyl (Boc).
17. The compound of any one of embodiments 1-15, wherein R^{1b} is allyl (Al).
18. The compound of any one of embodiments 1-15, wherein R^{1b} is trifluoroacetyl.
19. The compound of any one of embodiments 1-13, wherein R^{1a} and R^{1b} are taken together to form a cycloalkyl, heterocyclyl, aryl, or heteroaryl ring.
20. The compound of embodiment 19, wherein R^{1a} and R^{1b} are taken together to form a succinimidyl ring.
21. The compound of any one of embodiments 1-18, wherein R^{1a} is hydrogen.
22. The compound of any one of embodiments 1-18, wherein R^{1a} is hydrogen and R^{1b} is 9-fluorenylmethoxycarbonyl (Fmoc), benzyl (Bn), benzoyl (Bz), allyloxycarbonyl (Alloc), 2-(4-nitropheylsulfonyl)ethoxycarbonyl (Nsc), 1,1-dioxobenzo[b]thiophene-2-ylmethyloxycarbonyl (Bsmoc), 1,1-dioxonaphtho[1,2-b]thiophene (Nsmoc), 1-(4,4-dimethyl-2,6-dioxocyclohex-1-ylidene)-3-methylbutyl (ivDde), 2,2,2-trichloroethyloxycarbonyl (Troc), 2-[phenyl(methyl)sulfonio]ethyloxycarbonyl tetrafluoroborate (Pms), tert-butyloxycarbonyl (Boc), p-methoxybenzyl (PMB), carbobenzyloxy (Cbz), acetyl (Ac), trifluoroacetyl, tosyl (Ts), trityl (Trt), 2-(4-biphenyl)-isopropoxycarbonyl (Bpoc), allyl (Al), 2,2,5,5-tetramethyl-1,2,5-azadisilolidine, 1,3-dithiolan-2-imine, nitrobenzenesulfonyl (Nosyl), succinimidyl, and pyromellitic diimide.
23. The compound of any one of embodiments 1-18, wherein R^{1a} is hydrogen and R^{1b} is tert-butyloxycarbonyl (Boc).
24. The compound of any one of embodiments 1-18, wherein R^{1a} is hydrogen and R^{1b} is allyl (Al).
25. The compound of any one of embodiments 1-18, wherein R^{1a} is hydrogen and R^{1b} is trifluoroacetyl.
26. The compound of any one of embodiments 1-25, wherein each of R^{2a} and R^{2b} is independently hydrogen.
27. The compound of any one of embodiments 1-26, wherein each of R^{2c} and R^{2d} is independently hydrogen.
28. The compound of any one of embodiments 1-27, wherein each of R^{2a}, R^{2b}, R^{2c}, and R^{2d} is independently hydrogen.
29. The compound of any one of embodiments 1-28, wherein each of m and n is 1 or 2.
30. The compound of any one of embodiments 1-29, wherein q is 1, 2, 3, 4, 5, or 6.
31. The compound of any one of embodiments 1-30, wherein p is 0.
32. The compound of any one of embodiments 1-31, wherein the compound of Formula (I) is a compound of Formula (I-a):

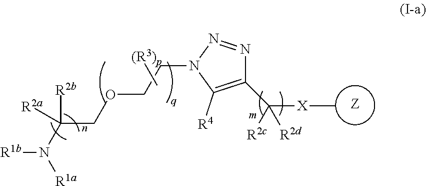

or a pharmaceutically acceptable salt thereof, wherein:
   Ring Z is cycloalkyl, heterocyclyl, aryl, or heteroaryl, each of which is optionally substituted with 1-6 R⁵;
   X is O, S, N(R^{A}), C₁-C₁₂ alkylene, C₁-C₁₂ alkenylene, C₂-C₁₂ heteroalkylene, or absent;
   R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group;
   R^{1b} is an amine-protecting group; or
   R^{1a} and R^{1b} may be taken together to form a cycloalkyl, heterocyclyl, aryl, or heteroaryl ring, each of which is optionally substituted with 1-6 R⁶;
   each of R^{2a}, R^{2b}, R^{2c}, and R^{2d} is independently hydrogen, C₁-C₁₂ alkyl, C₂-C₁₂ heteroalkyl, halogen, or OR^{C}; or
   R^{2a} and R^{2b} or R^{2c} and R^{2d} are taken together to form an oxo;
   each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
   m and n are each independently 0, 1, 2, 3, 4, 5, or 6;
   p is 0, 1, 2, 3, 4, or 5; and
   q is an integer from 0 to 25.
33. The compound of any one of embodiments 1-32, wherein the compound of Formula (I) is a compound of Formula (I-b):

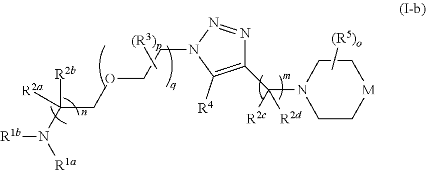

or a pharmaceutically acceptable salt thereof, wherein:
   M is C(R′)(R″), N(R′), or S(O)ₓ;
   each of R′ and R″ is independently hydrogen, C₁-C₁₂ alkyl, C₂-C₁₂ heteroalkyl, C₁-C₁₂ haloalkyl or halogen; or
   each of R′ and R″ is taken together to form an oxo;
   R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group;
   R^{1b} is an amine-protecting group; or
   R^{1a} and R^{1b} may be taken together to form a cycloalkyl, heterocyclyl, aryl, or heteroaryl ring, each of which is optionally substituted with 1-6 R⁶;
   each of R^{2a}, R^{2b}, R^{2c}, and R^{2d} is independently hydrogen, C₁-C₁₂ alkyl, C₂-C₁₂ heteroalkyl, or halogen; or
   R^{2a} and R^{2b} or R^{2c} and R^{2d} are taken together to form an oxo;
   each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
   m and n are each independently 0, 1, 2, 3, 4, 5, or 6;
   and p are each independently 0, 1, 2, 3, 4, or 5;
   q is an integer from 0 to 25; and
   x is 0, 1, or 2.
34. The compound of any one of embodiments 1-33, wherein the compound of Formula (I) is a compound of Formula (I-c):

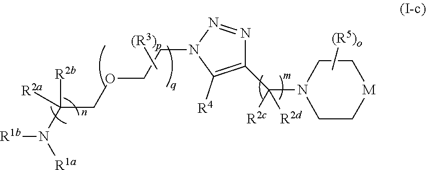

or a pharmaceutically acceptable salt thereof, wherein:
   M is C(R′)(R″), N(R′), or S(O)ₓ;
   each of R′ and R″ is independently hydrogen, C₁-C₁₂ alkyl, C₂-C₁₂ heteroalkyl, C₁-C₁₂ haloalkyl or halogen; or
   each of R′ and R″ is taken together to form an oxo;
   R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group;
   R^{1b} is an amine-protecting group; or
   R^{1a} and R^{1b} may be taken together to form a cycloalkyl, heterocyclyl, aryl, or heteroaryl ring, each of which is optionally substituted with 1-6 R⁶;
   each of R^{2a}, R^{2b}, R^{2c}, and R^{2d} is independently hydrogen, C₁-C₁₂ alkyl, C₂-C₁₂ heteroalkyl, or halogen; or
   R^{2a} and R^{2b} or R^{2c} and R^{2d} are taken together to form an oxo;
   each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
   m and n are each independently 0, 1, 2, 3, 4, 5, or 6;
   and p are each independently 0, 1, 2, 3, 4, or 5;
   q is an integer from 0 to 25; and
   x is 0, 1, or 2.
35. The compound of any one of embodiments 1-34, wherein the compound of Formula (I) is a compound of Formula (I-d):

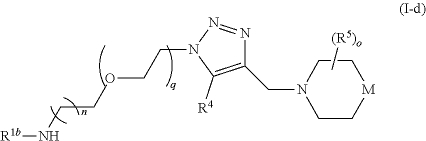

or a pharmaceutically acceptable salt thereof, wherein:
   M is C(R′)(R″), N(R′), or S(O)ₓ;
   each of R′ and R″ is independently hydrogen, C₁-C₁₂ alkyl, C₂-C₁₂ heteroalkyl, C₁-C₁₂ haloalkyl or halogen; or
   each of R′ and R″ is taken together to form an oxo;
   R^{1b} is an amine-protecting group;
   each R⁵ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B});
   each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
   n is 0, 1, 2, 3, 4, 5, or 6;
   is 0, 1, 2, 3, 4, or 5;
   q is an integer from 0 to 25; and
   x is 0, 1, or 2.
36. The compound of any one of embodiments 1-35, wherein the compound of Formula (I) is a compound of Formula (I-e):

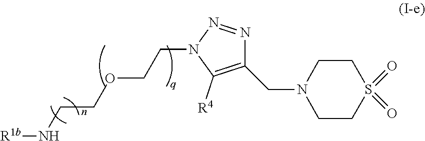

or a pharmaceutically acceptable salt thereof, wherein:
   R^{1b} is an amine-protecting group;
   R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B}
   n is 0, 1, 2, 3, 4, 5, or 6; and
   q is an integer from 0 to 25.
37. The compound of any one of embodiments 1-36, wherein the compound is a compound of Formula (I-f):

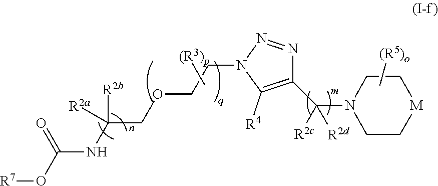

or a pharmaceutically acceptable salt thereof, wherein:
   M is C(R′)(R″), N(R′), or S(O)ₓ;
   each of R′ and R″ is independently hydrogen, C₁-C₁₂ alkyl, C₂-C₁₂ heteroalkyl, C₁-C₁₂ haloalkyl, or halogen; or
   each of R′ and R″ is taken together to form an oxo;
   R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   each R⁵ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   R⁷ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, cycloalkyl, heterocyclyl, aryl, or heteroaryl, wherein alkyl, alkenyl, alkynyl, heteroalkyl, haloalkyl, cycloalkyl, heterocyclyl, aryl, and heteroaryl is optionally substituted with 1-6 R⁵;
   each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
   m and n are each independently 0, 1, 2, 3, 4, 5, or 6;
   o and p are each independently 0, 1, 2, 3, 4, or 5;
   q is an integer from 0 to 25; and
   x is 0, 1, or 2.
38. The compound of any one of embodiments 1-37, wherein the compound of Formula (I) is a compound of Formula (I-g):

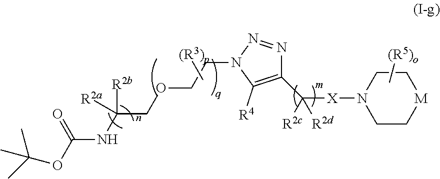

or a pharmaceutically acceptable salt thereof, wherein:
   M is C(R′)(R″), N(R′), or S(O)ₓ;
   each of R′ and R″ is independently hydrogen, C₁-C₁₂ alkyl, C₂-C₁₂ heteroalkyl, C₁-C₁₂ haloalkyl, or halogen; or
   each of R′ and R″ is taken together to form an oxo;
   X is O, S, N(R^{A}), C₁-C₁₂ alkylene, C₁-C₁₂ alkenylene, C₂-C₁₂ heteroalkylene, or absent;
   each of R^{2a}, R^{2b}, R^{2c}, and R^{2d} is independently hydrogen, C₁-C₁₂ alkyl, C₂-C₁₂ heteroalkyl, or halogen; or
   each of R^{2a}, R^{2b}, R^{2c}, and R^{2d} is independently hydrogen, C₁-C₁₂ alkyl, C₂-C₁₂ heteroalkyl, or halogen; or
   R^{2a} and R^{2b} or R^{2c} and R^{2d} are taken together to form an oxo;
   each of R³ and R⁵ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   each R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
   m and n are each independently 0, 1, 2, 3, 4, 5, or 6;
   o and p are each independently 0, 1, 2, 3, 4, or 5;
   q is an integer from 0 to 25; and
   x is 0, 1, or 2.
39. A compound of Formula (II):

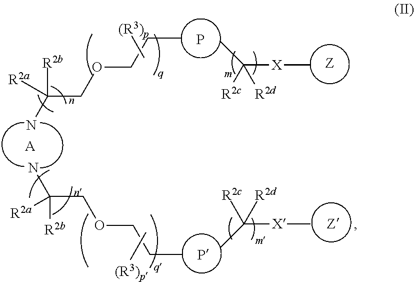

or a pharmaceutically acceptable salt thereof, wherein:
Rings P and P′ are each independently heterocyclyl or heteroaryl, each of which is optionally substituted with 1-6 R⁴;
Rings Z and Z′ are each independently cycloalkyl, heterocyclyl, aryl, or heteroaryl, each of which is optionally substituted with 1-6 R⁵;
Ring A is cycloalkyl, heterocyclyl, aryl, or heteroaryl, each of which is optionally substituted with 1-6 R⁶;
X and X′ are each independently O, S, N(R^{A}), C₁-C₁₂ alkylene, C₁-C₁₂ alkenylene, C₂-C₁₂ heteroalkylene, or absent;
each of R^{2a}, R^{2b}, R^{2c}, and R^{2d} is independently hydrogen, C₁-C₁₂ alkyl, C₂-C₁₂ heteroalkyl, halogen, or OR^{C}; or R^{2a} and R^{2b} or R^{2c} and R^{2d} are taken together to form an oxo;
each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B});
each R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
m, m′, n, and n′ are each independently 0, 1, 2, 3, 4, 5, or 6;
p and p′ are each independently 0, 1, 2, 3, 4, or 5; and
q and q′ are each independently an integer from 0 to 25.
40. The compound of embodiment 39, wherein each of Rings P and P′ is independently heteroaryl optionally substituted with 1-6 R⁴.
41. The compound of any one of embodiments 39-40, wherein each of Rings P and P′ is independently a five-membered heteroaryl ring.
42. The compound of any one of embodiments 39-41, wherein Ring P is 1,2,3-triazolyl (e.g.,

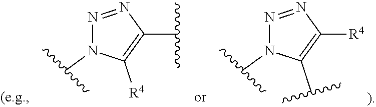

43. The compound of any one of embodiments 39-42, wherein Ring P′ is 1,2,3-triazolyl

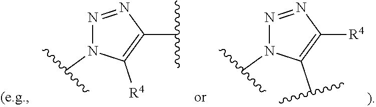

44. The compound of any one of embodiments 39-43, wherein Ring A is a tricyclic heterocyclyl.

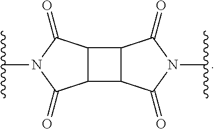 [SMILES: *N1C(=O)C2C(C1=O)C1C(=O)N(*)C(=O)C21]

45. The compound of any one of embodiments 39-44, wherein Ring A is O.
46. The compound of any one of embodiments 39-45, wherein each of Rings Z and Z′ is independently heterocyclyl optionally substituted with 1-6 R⁴.
47. The compound of any one of embodiments 39-46, wherein Ring Z is thiomorpholinyl-1,1-dioxidyl

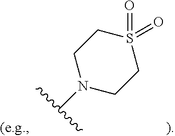 [SMILES: *N1CCS(=O)(=O)CC1]

48. The compound of any one of embodiments 39-47, wherein Ring Z′ is thiomorpholinyl-1,1-dioxidyl

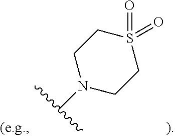 [SMILES: *N1CCS(=O)(=O)CC1]

49. The compound of any one of embodiments 39-48, wherein X and X′ are absent or O.
50. The compound of any one of embodiments 39-49, wherein each of X and X′ is absent.
51. The compound of any one of embodiments 39-50, wherein each of R^{2a} and R^{2b} is independently hydrogen.
52. The compound of any one of embodiments 39-51, wherein each of R^{2c} and R^{2d} is independently hydrogen.
53. The compound of any one of embodiments 39-52, wherein each of R^{2a}, R^{2b}, R^{2c}, and R^{2d} is independently hydrogen.
54. The compound of any one of embodiments 39-53, wherein each of m and m′ is 1 or 2.
55. The compound of any one of embodiments 39-54, wherein each of n and n′ is 1 or 2.
56. The compound of any one of embodiments 39-55, wherein each of q and q′ is 1, 2, 3, 4, 5, or 6.
57. The compound of any one of embodiments 39-56, wherein each of p and p′ is 0.
58. A compound of Formula (III):

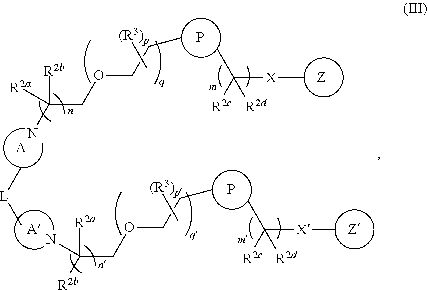

or a pharmaceutically acceptable salt thereof, wherein:
Rings A and A′ are each independently heterocyclyl or heteroaryl, each of which is optionally substituted with 1-6 R⁶;
L is —O—, —C(O)—, —N(R^{A})—, —S(O)ₓ—, C₁-C₁₂ alkylene, C₁-C₁₂ alkenylene, C₂-C₁₂ heteroalkylene, C₁-C₁₂ haloalkylene, or absent, wherein each alkylene, alkenylene, heteroalkylene, and haloalkylene is optionally substituted with 1-6 R⁵;
Rings P and P′ are each independently heterocyclyl or heteroaryl, each of which is optionally substituted with 1-6 R⁴;
Rings Z and Z′ are each independently cycloalkyl, heterocyclyl, aryl, or heteroaryl, each of which is optionally substituted with 1-6 R⁵;
X and X′ are each independently O, S, N(R^{A}), C₁-C₁₂ alkylene, C₁-C₁₂ alkenylene, C₂-C₁₂ heteroalkylene, or absent;
each of R^{2a}, R^{2b}, R^{2c}, and R^{2d} is independently hydrogen, C₁-C₁₂ alkyl, C₂-C₁₂ heteroalkyl, halogen, or OR^{C}; or R^{2a} and R^{2b} or R^{2c} and R^{2d} are taken together to form an oxo;
each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B});
each R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
m, m′, n, and n′ are each independently 0, 1, 2, 3, 4, 5, or 6;
p and p′ are each independently 0, 1, 2, 3, 4, or 5;
q and q′ are each independently an integer from 0 to 25; and
x is 0, 1, or 2.
59. The compound of embodiment 58, wherein each of Rings A and A¹ is independently heteroaryl or heterocyclyl optionally substituted with 1-6 R⁶.
60. The compound of any one of embodiments 58-59, wherein each of Rings A and A′ is independently phthalimidyl optionally substituted with 1-6 R⁶.
61. The compound of any one of embodiments 58-60, wherein each of Rings P and P′ is independently heteroaryl optionally substituted with 1-6 R⁴.
62. The compound of any one of embodiments 58-61, wherein each of Rings P and P′ is independently a five-membered heteroaryl ring.
63. The compound of any one of embodiments 58-62, wherein Ring P is 1,2,3-triazolyl

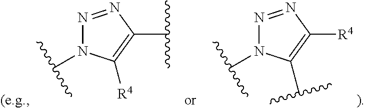

64. The compound of any one of embodiments 58-63, wherein Ring P′ is 1,2,3-triazolyl

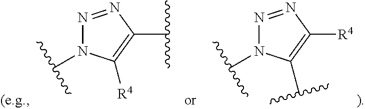

65. The compound of any one of embodiments 58-64, wherein L is absent, —O—, —C(O)—, —N(R^{A})—, —S(O)ₓ—, or C₁-C₁₂ alkylene, wherein alkylene is optionally substituted with 1-6 R⁵
66. The compound of any one of embodiments 58-65, wherein L is absent.
67. The compound of any one of embodiments 58-66, wherein each of Rings Z and Z′ is independently heterocyclyl optionally substituted with 1-6 R⁴.
68. The compound of any one of embodiments 58-67, wherein Ring Z is thiomorpholinyl-1,1-dioxidyl

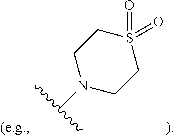 [SMILES: *N1CCS(=O)(=O)CC1]

69. The compound of any one of embodiments 58-68, wherein Ring Z′ is thiomorpholinyl-1,1-dioxidyl

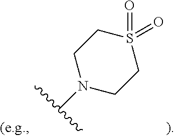 [SMILES: *N1CCS(=O)(=O)CC1]

70. The compound of any one of embodiments 58-69, wherein X and X′ are absent or 0.
71. The compound of any one of embodiments 58-70, wherein each of X and X′ is absent.
72. The compound of any one of embodiments 58-71, wherein each of R^{2a} and R^{2b} is independently hydrogen.
73. The compound of any one of embodiments 58-72, wherein each of R^{2c} and R^{2d} is independently hydrogen.
74. The compound of any one of embodiments 58-73, wherein each of R^{2a}, R^{2b}, R^{2c}, and R^{2d} is independently hydrogen.
75. The compound of any one of embodiments 58-74, wherein each of m and m′ is 1 or 2.
76. The compound of any one of embodiments 58-75, wherein each of n and n′ is 1 or 2.
77. The compound of any one of embodiments 58-76, wherein each of q and q′ is 1, 2, 3, 4, 5, or 6.
78. The compound of any one of embodiments 58-77, wherein each of p and p′ is 0.
79. The compound of any one of embodiments 1-78, wherein the compound is selected from a compound shown in Table 1 or a pharmaceutically acceptable salt thereof.
80. The compound of any one of embodiments 1-79, wherein the onset temperature of the compound is greater than about 150° C. (e.g., greater than about 175° C., 200° C., 225° C., 250° C., 275° C., 300° C., or 325° C.).
81. The compound of any one of embodiments 1-80, wherein the onset temperature of the compound is between 150° C. and 350° C. (e.g., between 200° C. and 350° C. or between 250° C. and 350° C.).
82. The compound of any one of embodiments 1-81, wherein the maximum exothermal output of the compound is lower than about 750 J/g (e.g., lower than about 700 J/g, 650 J/g, 600 J/g, 550 J/g, 500 J/g, or 450 J/g).
83. The compound of any one of embodiments 1-82, wherein the maximum exothermal output of the compound is between about 100 J/g and 700 J/g (e.g., 200 J/g and 600 J/g or between 250 J/g and 550 J/g).
84. The compound of any one of embodiments 1-83, wherein the maximum exothermal output of the compound is lower than about 300 kJ/mol, e.g., lower than about 250 kJ/mol, 225 kJ/mol, 200 kJ/mol, and 700 J/g (e.g., 200 J/g and 600 J/g or between 250 J/g and 550 J/g).
85. The compound of any one of embodiments 1-84, wherein the compound is a liquid.
86. A composition comprising a compound of any one of embodiments 1-85.
87. A container comprising greater that about 5 grams, 10 grams, 25 grams, 50 grams, 75 grams, 100 grams, 250 grams, 500 grams, 750 grams, 1 kilogram, 2.5 kilograms, 5 kilograms, 10 kilograms, 20 kilograms, or more of a compound of Formula (I), (II), or (III) or a pharmaceutically acceptable salt thereof according to any one of embodiments 1-85, or a composition thereof according to embodiment 86.
88. The container of embodiment 87, wherein the container is a vial.
89. The container of any one of embodiments 87-88, wherein the container comprises glass, steel, or plastic.
90. The container of any one of embodiments 87-89, wherein the container is airtight.
91. A preparation comprising greater that about 5 grams, 10 grams, 25 grams, 50 grams, 75 grams, 100 grams, 250 grams, 500 grams, 750 grams, 1 kilogram, 2.5 kilograms, 5 kilograms, 10 kilograms, 20 kilograms, or more of a compound of Formula (I), (II), or (III) or a pharmaceutically acceptable salt thereof according to any one of embodiments 1-85, or a composition thereof according to embodiment 86.
92. The preparation of claim 91, wherein the preparation is substantially pure.
93. The preparation of any one of embodiments 91-92, wherein the preparation comprises less than 25%, 20%, 15%, 12.5%1, 0%. 9%, 8%, 7%, 6%, 5%, 4%, 3%, 2%, 1%, 0.5% of a second compound (e.g., an intermediate in the method of making the compound of Formula (I), (II), or (III)).
94. The preparation of any one of embodiments 91-93, wherein the second compound is a compound selected from a compound shown in Table 2.
95. A compound selected from a compound shown in Table 2 (e.g., Compound 200, 201, 202, 203, 204, 205, 205, 206, 207, or a pharmaceutically acceptable salt thereof).
96. A method of preparing a compound of Formula (I):

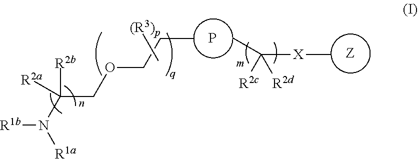

or a pharmaceutically acceptable salt thereof, wherein:
   Ring P is heterocyclyl or heteroaryl, each of which is optionally substituted with 1-6 R⁴;
   Ring Z is cycloalkyl, heterocyclyl, aryl, or heteroaryl, each of which is optionally substituted with 1-6 R⁵;
   X is O, S, N(R^{A}), C₁-C₁₂ alkylene, C₁-C₁₂ alkenylene, C₂-C₁₂ heteroalkylene, or absent;
   R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group;
   R^{1b} is an amine-protecting group; or
   R^{1a} and R^{1b} may be taken together to form a cycloalkyl, heterocyclyl, aryl, or heteroaryl ring, each of which is optionally substituted with 1-6 R⁶;
   each of R^{2a}, R^{2b}, R^{2c}, and R^{2d} is independently hydrogen, C₁-C₁₂ alkyl, C₂-C₁₂ heteroalkyl, halogen, or OR^{C}; or
   R^{2a} and R^{2b} or R^{2c} and R^{2d} are taken together to form an oxo;
   each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   each R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
   m and n are each independently 0, 1, 2, 3, 4, 5, or 6;
   p is 0, 1, 2, 3, 4, or 5; and
   q is an integer from 0 to 25;
wherein the method comprises reacting a compound of Formula (V):

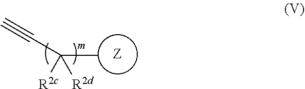

with a compound of Formula (VI):

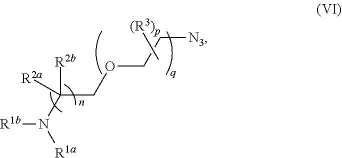

thereby preparing a compound of Formula (I).
97. The method of embodiment 96, wherein the reaction of a compound of Formula (V) with a compound of Formula (VI) occurs in the presence of a catalyst (e.g., a copper catalyst).
98. The method of any one of embodiments 96-97, wherein the catalyst comprises a CuSO₄ hydrate.
99. The method of any one of embodiments 96-98, wherein the onset temperature of the compound of Formula (I) is greater than about 150° C. (e.g., greater than about 175° C., 200° C., 225° C., 250° C., 275° C., 300° C., or 325° C.).
100. The method of any one of embodiments 96-99, wherein the onset temperature of the compound of Formula (I) is between about than about 150° C. and 350° C. (e.g., between 200° C. and 350° C. or between 250° C. and 350° C.).
101. The method of any one of embodiments 96-100, wherein the reaction is carried out at a temperature greater than about 25° C. (e.g., greater than about 30° C., 35° C., 40° C., 45° C., 50° C., 55° C., 60° C., 65° C., 70° C., 75° C., 80° C., 85° C., 90° C. or higher).
102. The method of any one of embodiments 96-101, wherein the maximum exothermal output of the compound of Formula (I) is lower than about 750 J/g (e.g., lower than about 700 J/g, 650 J/g, 600 J/g, 550 J/g, 500 J/g, or 450 J/g).
103. The method of any one of embodiments 96-101, wherein the maximum exothermal output of the compound of Formula (I) is between about 100 J/g and 700 J/g (e.g., 200 J/g and 600 J/g or between 250 J/g and 550 J/g).
104. The method of any one of embodiments 96-103, further comprising providing reacting at least 1 g (e.g., 2 g, 5 g, 10 g, 25 g, 50 g, 75 g, 100 g, 250 g, 500 g, 750 g, 1 kg, 2 kg, or more) of Formula (V) with 1 or more (e.g., 1.1, 1.2, 1.3, 1.4, 1.5, 1.6, 1.7, 1.8, 1.9, 2, 2.25, 2.5, 3, 3.5, 4, 5, 7.5, or more) equivalents of Formula (VI).
105. The method of any one of embodiments 96-104, wherein the compound of Formula (I) is a compound of Formula (I-c):

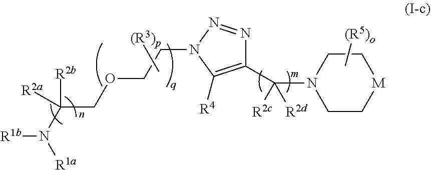

or a pharmaceutically acceptable salt thereof, wherein:
   M is C(R′)(R″), N(R′), or S(O)ₓ;
   each of R′ and R″ is independently hydrogen, C₁-C₁₂ alkyl, C₂-C₁₂ heteroalkyl, C₁-C₁₂ haloalkyl or halogen; or
   each of R′ and R″ is taken together to form an oxo;
   R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group;
   R^{1b} is an amine-protecting group; or
   R^{1a} and R^{1b} may be taken together to form a cycloalkyl, heterocyclyl, aryl, or heteroaryl ring, each of which is optionally substituted with 1-6 R⁶;
   each of R^{2a}, R^{2b}, R^{2c}, and R^{2d} is independently hydrogen, C₁-C₁₂ alkyl, C₂-C₁₂ heteroalkyl, or halogen; or
   R^{2a} and R^{2b} or R^{2c} and R^{2d} are taken together to form an oxo;
   each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
   each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
   m and n are each independently 0, 1, 2, 3, 4, 5, or 6;
   and p are each independently 0, 1, 2, 3, 4, or 5;
   q is an integer from 0 to 25; and
   x is 0, 1, or 2;
wherein the method comprises reacting a compound of Formula (V-a):

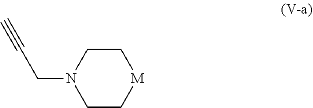

with a compound of Formula (VI-a):

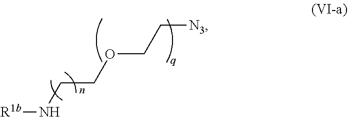

thereby preparing a compound of Formula (I).
106. A preparation of a compound of Formula (I), (II), or (III) described herein comprising a second compound in an amount less than about 25%, 20%, 15%, 12.5%, 10%. 9%, 8%, 7%, 6%, 5%, 4%, 3%, 2%, 1%, 0.75%, 0.5%, 0.25%, 0.1%, or 0.05% (w/w, w/v, v/v, or % by dry weight) of the preparation.
107. The preparation of embodiment 106, wherein the second compound is present in an amount less than 10% (w/w, w/v, v/v, or % by dry weight) of the preparation.
108. The preparation of embodiment 106, wherein the second compound is present in an amount less than 5% (w/w, w/v, v/v, or % by dry weight) of the preparation.
109. The preparation of embodiment 106, wherein the second compound is present in an amount less than 2.5% (w/w, w/v, v/v, or % by dry weight) of the preparation.
110. The preparation of embodiment 106, wherein the second compound is present in an amount less than 1% (w/w, w/v, v/v, or % by dry weight) of the preparation.
111. The preparation of embodiment 106, wherein the second compound is present in an amount less than 0.75% (w/w, w/v, v/v, or % by dry weight) of the preparation.
112. The preparation of embodiment 106, wherein the second compound is present in an amount less than 0.5% (w/w, w/v, v/v, or % by dry weight) of the preparation.
113. The preparation of any one of embodiments 106-112, wherein the second compound is selected from a compound in Table 2.
114. A composition of a compound of Formula (I), (II), or (III) described herein comprising a second compound in an amount less than about 25%, 20%, 15%, 12.5%, 10%. 9%, 8%, 7%, 6%, 5%, 4%, 3%, 2%, 1%, 0.75%, 0.5%, 0.25%, 0.1%, or 0.05% (w/w, w/v, v/v, or % by dry weight) of the composition.
115. The composition of embodiment 114, wherein the second compound is present in an amount less than 10% (w/w, w/v, v/v, or % by dry weight) of the composition.
116. The composition of embodiment 114, wherein the second compound is present in an amount less than 5% (w/w, w/v, v/v, or % by dry weight) of the composition.
117. The composition of embodiment 114, wherein the second compound is present in an amount less than 2.5% (w/w, w/v, v/v, or % by dry weight) of the composition.
118. The composition of embodiment 114, wherein the second compound is present in an amount less than 1% (w/w, w/v, v/v, or % by dry weight) of the composition.
119. The composition of embodiment 114, wherein the second compound is present in an amount less than 0.5% (w/w, w/v, v/v, or % by dry weight) of the composition.
120. The composition of any one of embodiments 114-119, wherein the second compound is selected from a compound in Table 2.

### EXAMPLES

In order that the invention described herein may be more fully understood, the following examples are set forth. The procedures described in this application are offered to illustrate the compounds, compositions, and methods provided herein and are not to be construed in any way as limiting their scope.

The compounds, compositions, and methods thereof provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

### Example 1: Synthesis of Exemplary Intermediates of Compounds of Formulas (I) and (II)

### General Methods:

### Method A

To a vial fitted with a magnetic stirrer and septum screw cap were added base (1.2 equiv), a primary amine to be protected (1.0 equiv), and dichloromethane (5 volumes). The mixture was cooled to <5° C. and then an amine-protecting group reagent (1.1 equiv) was added. The reaction mixture was allowed to warm to room temperature and stir for 16 hours. The protected amine mixture was filtered, concentrated, and purified by silica gel chromatography (0-10 methanol in dichloromethane). Product-containing fractions were combined and concentrated to afford the desired product.

### Method B

To a vial fitted with a magnetic stirrer were added primary amine to be protected (1.0 equiv) and 1N sodium hydroxide in water (5 volumes). The resulting solution was cooled to 0° C. and carbon disulfide (1.01 equiv) was added dropwise. The resulting solution was allowed to warm to room temperature and stirred for 5 minutes. After 5 minutes, the solution was cooled to 0° C. and 1,2-dibromoethane (1.01 equiv) was added dropwise. The resulting bilayer was stirred, and the mixture was allowed to warm to room temperature and stir for 16 hours. The reaction mixture was filtered, concentrated, and purified by silica gel chromatography (0-10% methanol in dichloromethane). Product-containing fractions were combined and concentrated to afford the desired product.

### Method C

The anhydride (1.0 equiv) and primary amine to be protected (1.05 equiv) were refluxed in anhydrous toluene (30 volumes) for 18 h while removing any water generated through the use of a Dean-Stark apparatus. The resulting mixture was cooled to room temperature, then filtered, concentrated, and purified by silica gel chromatography (0-10% methanol in dichloromethane). Product-containing fractions were combined and concentrated to afford the desired product.

### Method D

The primary amine to be protected (1.17 equiv) was dissolved in methanol (16 volumes) and water (1 volume), then sodium L-ascorbate (0.10 equiv), cuprous iodide (0.10 equiv), trans-N,N-dimethylcyclohexane-1,2-diamine (0.13 equiv), and thiomorpholine-1,1-dioxide (1.0 equiv) were added. The reaction mixture was heated to 55° C. at stirred for 16 hours. The resulting reaction mixture was concentrated and purified by silica gel chromatography (0-20% methanol in dichloromethane). Product-containing fractions were combined and concentrated to afford the desired product.

### Azide Intermediate for Compound 101: 2-(2-(2-(2-azidoethoxy)ethoxy)ethoxy)-N-benzylethan-1-amine

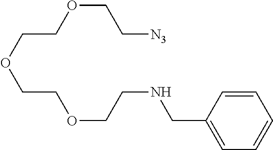 [SMILES: [N-]=[N+]=NCCOCCOCCOCCNCc1ccccc1]

Method A; 1H NMR (500 MHz, CDCl3) δ 7.62-7.57 (m, 2H), 7.40-7.30 (m, 3H), 4.20 (s, 2H), 3.93-3.83 (m, 2H), 3.64 (s, 4H), 3.59 (dtd, J=6.9, 4.2, 3.4, 1.7 Hz, 6H), 3.33 (dd, J=10.0, 4.9 Hz, 2H), 3.14-3.02 (m, 2H); 13C NMR (126 MHz, CDCl3) δ 130.4, 130.3, 129.3, 129.0, 70.3, 70.2, 70.2, 69.8, 65.8, 51.0, 50.6, 45.6; [M+H]+ calc'd 309.1921, found 309.1321.

### Azide Intermediate for Compound 103: N-allyl-N-(2-(2-(2-(2-azidoethoxy)ethoxy)ethoxy)ethyl)prop-2-en-1-amine

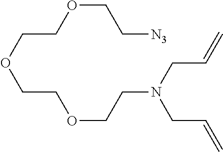 [SMILES: C=CCN(CC=C)CCOCCOCCOCCN=[N+]=[N-]]

Method A (except 2.2 equiv. of base and 2.1 equiv. of protecting group reagent); 1H NMR (500 MHz, CDCl3) δ 5.82 (ddt, J=16.8, 10.2, 6.5 Hz, 2H), 5.18-5.06 (m, 4H), 3.67-3.50 (m, 14H), 3.39-3.32 (m, 2H), 3.11 (d, J=6.5 Hz, 4H), 2.64 (t, J=6.2 Hz, 2H); 13C NMR (126 MHz, CDCl3) δ 135.6, 117.4, 70.6, 70.6, 70.6, 69.9, 69.6, 57.4, 52.4, 50.6; [M+H]+ calc'd 299.2078, found 299.2078.

### Azide Intermediate for Compound 102: N-(2-(2-(2-(2-azidoethoxy)ethoxy)ethoxy)ethyl)-2,2,2-trifluoroacetamide

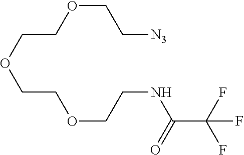 [SMILES: [N-]=[N+]=NCCOCCOCCOCCNC(=O)C(F)(F)F]

Method A; 1H NMR (400 MHz, CDCl3) δ 7.18 (s, 1H), 3.62 (d, J=10.6 Hz, 12H), 3.52 (q, J=5.1 Hz, 2H), 3.36 (t, J=4.9 Hz, 2H); 13C NMR (101 MHz, CDCl3) δ 157.2 (q, J=36.9 Hz), 115.8 (q, J=287.5 Hz), 70.5, 70.5, 70.4, 70.2, 69.9, 68.5, 50.6, 39.6; 19F NMR (377 MHz, CDCl3) δ −75.9; [M+H]+ calc'd 315.1275, found 315.1274.

### Azide Intermediate for Compound 107: (9H-fluoren-9-yl)methyl (2-(2-(2-(2-azidoethoxy)ethoxy)ethoxy)ethyl)carbamate

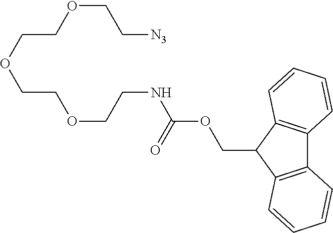 [SMILES: [N-]=[N+]=NCCOCCOCCOCCNC(=O)OCC1c2ccccc2-c2ccccc21]

Method A; 1H NMR (500 MHz, CDCl3) δ 7.76 (d, J=7.5 Hz, 2H), 7.60 (d, J=7.4 Hz, 2H), 7.39 (t, J=7.5 Hz, 2H), 7.31 (t, J=7.4 Hz, 2H), 5.40 (s, 1H), 4.41 (d, J=7.0 Hz, 2H), 4.22 (t, J=6.9 Hz, 1H), 3.68-3.59 (m, 11H), 3.59-3.53 (m, 2H), 3.39 (q, J=5.3 Hz, 2H), 3.34 (t, J=5.0 Hz, 2H); 13C NMR (126 MHz, CDCl3) δ 156.4, 143.9, 141.2, 127.6, 127.0, 126.9, 125.0, 119.9, 70.6, 70.5, 70.5, 70.2, 69.9, 66.4, 50.5, 47.2, 40.8; [M+H]+ calc'd 441.2132, found 441.2131.

### Azide Intermediate for Compound 109: 2,5-bis(2-(2-(2-(2-azidoethoxy)ethoxy)ethoxy)ethyl)tetrahydrocyclobuta[1,2-c:3,4-c]dipyrrole-1,3,4,6(2H,5H)-tetraone

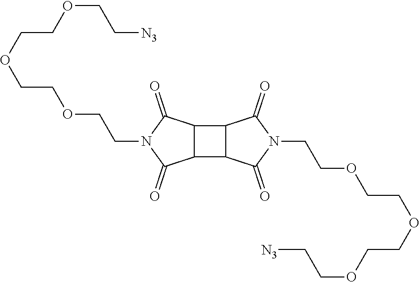 [SMILES: [N-]=[N+]=NCCOCCOCCOCCN1C(=O)C2C(C1=O)C1C(=O)N(CCOCCOCCOCCN=[N+]=[N-])C(=O)C21]

Method C (Except with double the amount of primary amine); 1H NMR (400 MHz, CDCl3) δ 3.79 (t, J=5.4 Hz, 4H), 3.69 (t, J=5.4 Hz, 4H), 3.67-3.51 (m, 20H), 3.40 (s, 4H), 3.36 (t, J=5.0 Hz, 4H); 13C NMR (101 MHz, CDCl3) δ 175.0, 70.6, 70.5, 70.4, 69.9, 69.8, 66.4, 50.6, 41.4, 38.4; [M+H]+ calc'd 597.2627, found 597.2627.

### Azide Intermediate for Compound 104: 1-(2-(2-(2-(2-azidoethoxy)ethoxy)ethoxy)-ethyl)pyrrolidine-2,5-dione

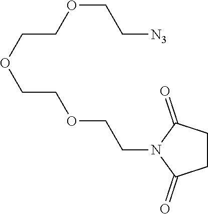 [SMILES: [N-]=[N+]=NCCOCCOCCOCCN1C(=O)CCC1=O]

Method C; 1H NMR (500 MHz, CDCl3) δ 3.65 (t, J=5.8 Hz, 2H), 3.62-3.56 (m, 8H), 3.54 (s, 4H), 3.32 (t, J=5.0 Hz, 2H), 2.64 (s, 4H); 13C NMR (126 MHz, CDCl3) δ 177.0, 70.4, 70.4, 69.8, 69.7, 66.8, 50.5, 37.7, 27.9; [M+H]+ calc'd 301.1506, found 301.1509.

### Azide Intermediate for Compound 105: N-(2-(2-(2-(2-azidoethoxy)ethoxy)ethoxy)ethyl)-1,3-dithiolan-2-imine

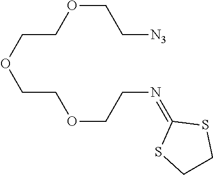 [SMILES: [N-]=[N+]=NCCOCCOCCOCCN=C1SCCS1]

Method B; 1H NMR (500 MHz, CDCl3) δ 3.72 (t, J=6.1 Hz, 1H), 3.68-3.60 (m, 6H), 3.55 (dd, J=7.0, 5.3 Hz, 1H), 3.45 (t, J=6.1 Hz, 1H), 3.41-3.35 (m, 2H); 13C NMR (126 MHz, CDCl3) δ 169.2, 70.7, 70.6, 70.5, 70.4, 69.9, 58.6, 50.6, 37.7, 34.7; [M+H]+ calc'd 321.105, found 321.1048.

### Azide Intermediate for Compound 106: N-(2-(2-(2-(2-azidoethoxy)ethoxy)ethoxy)ethyl)-4-nitrobenzenesulfonamide

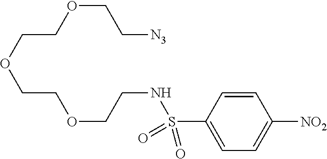 [SMILES: [N-]=[N+]=NCCOCCOCCOCCNS(=O)(=O)c1ccc([N+](=O)[O-])cc1]

Method A; 1H NMR (500 MHz, CDCl3) δ 8.33 (d, J=8.8 Hz, 2H), 8.05 (d, J=8.8 Hz, 2H), 5.71 (t, J=5.8 Hz, 1H), 3.68-3.65 (m, 4H), 3.63 (dd, J=5.7, 2.3 Hz, 2H), 3.59-3.56 (m, 2H), 3.52 (dd, J=5.6, 2.8 Hz, 2H), 3.50 (t, J=5.0 Hz, 2H), 3.37 (t, J=5.0 Hz, 2H), 3.17 (q, J=5.4 Hz, 2H); 13C NMR (126 MHz, CDCl3) δ 149.9, 146.1, 128.2, 124.2, 70.5, 70.4, 70.4, 70.1, 69.9, 68.9, 50.5, 43.0; [M+H]+ calc'd 404.1234, found 404.1234.

### Azide Intermediate for Compound 112: 2-(2-(2-(2-azidoethoxy)ethoxy)ethoxy)-N,N-dibenzylethan-1-amine

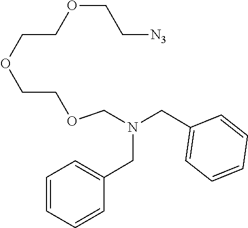 [SMILES: [N-]=[N+]=NCCOCCOCCOCN(Cc1ccccc1)Cc1ccccc1]

Method A; 1H NMR (500 MHz, CDCl3) δ 7.39-7.37 (m, 4H), 7.32-7.29 (m, 4H), 7.24-7.21 (m, 2H), 3.69-3.54 (m, 18H), 3.34 (t, J=5.0 Hz, 2H); 13C NMR (126 MHz, CDCl3) δ 139.5, 128.5, 128.0, 126.0, 70.3, 70.2, 69.8, 58.6, 52.5, 51.0; [M+H]+ calc'd 399.2396, found 399.2391.

### Azide Intermediate for Compound 111: 2-(2-(2-(2-(2-azidoethoxy)ethoxy)ethoxy)ethyl)-4,5,6,7-tetrachloroisoindoline-1,3-dione

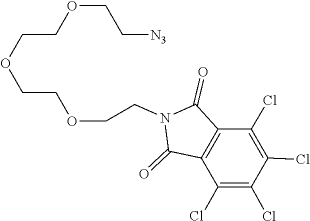 [SMILES: [N-]=[N+]=NCCOCCOCCOCCN1C(=O)c2c(Cl)c(Cl)c(Cl)c(Cl)c2C1=O]

Method C; 1H NMR (500 MHz, CDCl3) δ 3.90 (t, J=7.0 Hz, 2H), 3.73 (t, J=7.0 Hz, 2H), 3.66-3.60 (m, 10H), 3.36 (t, J=6.5 Hz, 2H); 13C NMR (126 MHz, CDCl3) δ 163.4, 140.0, 129.5, 127.6, 70.6, 70.0, 69.9, 67.4, 50.6, 38.0; [M+H]+ calc'd 484.9948, found 484.9948.

### Azide Intermediate for Compound 113: 2-(2-(2-(2-(2-azidoethoxy)ethoxy)ethoxy)ethyl)-4,5,6,7-tetrabromoisoindoline-1,3-dione

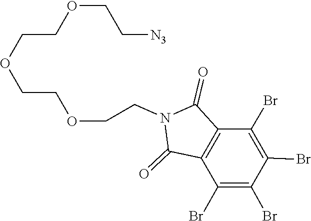 [SMILES: [N-]=[N+]=NCCOCCOCCOCCN1C(=O)c2c(Br)c(Br)c(Br)c(Br)c2C1=O]

Method C; 1H NMR (500 MHz, CDCl3) δ 3.91 (t, J=7.0 Hz, 2H), 3.74 (t, J=7.0 Hz, 2H), 3.68-3.60 (m, 10H), 3.36 (t, J=6.5 Hz, 2H); 13C NMR (126 MHz, CDCl3) δ 163.6, 137.4, 130.6, 121.2, 70.6, 70.6, 70.0, 69.9, 67.3, 50.6, 38.2; [M+H]+ calc'd 660.7927, found 660.7911.

### Azide Intermediate for Compound 115: 2,7-bis(2-(2-(2-(2-azidoethoxy)ethoxy)ethoxy)ethyl)benzo[lmn][3,8]phenanthroline-1,3,6,8(2H, 7H)-tetraone

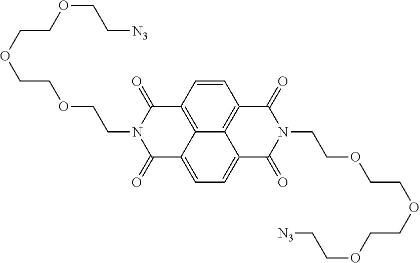 [SMILES: [N-]=[N+]=NCCOCCOCCOCCN1C(=O)c2ccc3c4c(ccc(c24)C1=O)C(=O)N(CCOCCOCCOCCN=[N+]=[N-])C3=O]

Method C; 1H NMR (500 MHz, CDCl3) δ 8.69 (s, 4H), 4.42 (t, J=6.0 Hz, 4H), 3.82 (t, J=6.0 Hz, 4H), 3.68-3.56 (m, 20H), 3.33-3.31 (m, 4H); 13C NMR (126 MHz, CDCl3) δ 162.8, 130.9, 126.6, 126.5, 70.7, 70.6, 70.1, 70.0, 67.8, 50.6, 39.6; [M+H]+ calc'd 669.2627, found 669.2627.

### Azide Intermediate for Compound 116:2,9-bis(2-(2-(2-(2-azidoethoxy)ethoxy)ethoxy)ethyl)-5,6,12,13-tetrachloroanthra[2,1,9-def-6,5,10-def]diisoquinoline-1,3,8,10(2H, 9H)-tetraone

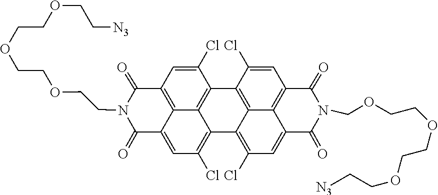 [SMILES: [N-]=[N+]=NCCOCCOCCOCCN1C(=O)c2cc(Cl)c3c4c(Cl)cc5c6c(cc(Cl)c(c7c(Cl)cc(c2c37)C1=O)c64)C(=O)N(COCCOCCOCCN=[N+]=[N-])C5=O]

Method C; 1H NMR (500 MHz, CDCl3) δ 8.66 (s, 4H), 4.51-4.40 (m, 4H), 4.42 (h, J=7.0 Hz, 4H), 3.82 (t, J=6.0 Hz, 4H), 3.71-3.57 (m, 20H), 3.34 (t, J=6.5 Hz, 4H); 13C NMR (126 MHz, CDCl3) δ 162.2, 135.3, 132.9, 131.4, 128.6, 123.3, 123.1, 70.6, 70.6, 70.6, 70.1, 69.9, 67.8, 50.6, 39.5; [M+H]+ calc'd 929.1381, found 929.1381.

### Azide Intermediate for Compound 110: 2-(2-(2-(2-(2-azidoethoxy)ethoxy)ethoxy)ethyl)-6-bromo-1H-benzo[de]isoquinoline-1,3(2H)-dione

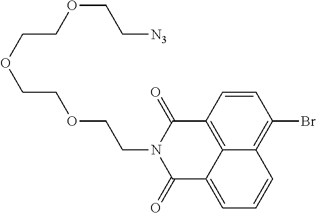 [SMILES: [N-]=[N+]=NCCOCCOCCOCCN1C(=O)c2cccc3c(Br)ccc(c23)C1=O]

Method C; 1H NMR (500 MHz, CDCl3) δ 8.63 (d, J=9.0 Hz, 1H), 8.55 (d, J=10.5 Hz, 1H), 8.39 (d, J=9.5 Hz, 1H), 8.02 (d, J=10.0 Hz, 1H), 7.85-7.81 (m, 1H), 4.42 (t, J=7.5 Hz, 2H), 3.82 (t, J=7.5 Hz, 2H), 3.70-3.54 (m, 10H), 3.34 (t, J=6.5 Hz, 2H); 13C NMR (126 MHz, CDCl3) δ 163.6, 133.2, 132.0, 131.2, 131.0, 130.6, 130.3, 129.0, 128.0, 123.0, 122.2, 70.6, 70.6, 70.1, 69.9, 67.8, 50.6, 39.2; [M+H]+ calc'd 477.0768, found 477.0762.

### Azide Intermediate for Compound 114: 2,2-bis(2-(2-(2-(2-azidoethoxy)ethoxy)ethoxy)ethyl)-[5,5-biisoindoline]-1,1,3,3-tetraone

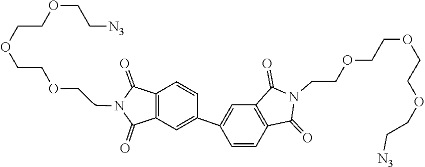 [SMILES: [N-]=[N+]=NCCOCCOCCOCCN1C(=O)c2ccc(-c3ccc4c(c3)C(=O)N(CCOCCOCCOCCN=[N+]=[N-])C4=O)cc2C1=O]

Method C; 1H NMR (500 MHz, CDCl3) δ 8.08 (s, 2H), 7.95 (s, 4H), 3.92 (t, J=7.5 Hz, 4H), 3.75 (t, J=7.0 Hz, 4H), 3.65-3.59 (m, 20H), 3.35 (t, J=6.5 Hz, 4H); 13C NMR (126 MHz, CDCl3) δ 145.2, 135.3, 132.9, 131.4, 124.3, 120.1, 70.5, 70.5, 70.0, 69.9, 67.5, 50.6, 39.5; [M+H]+ calc'd 695.2784, found 695.2783.

### Azide Intermediate for Compound 117: 5,5-oxybis(2-(2-(2-(2-(2-azidoethoxy)ethoxy)ethoxy-ethyl)isoindoline-1,3-dione)

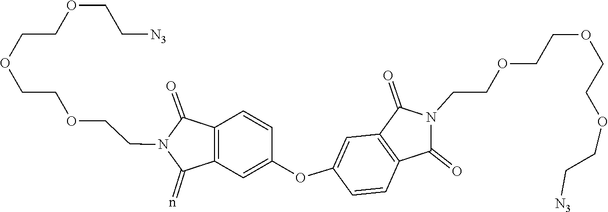

Method C; 1H NMR (500 MHz, CDCl3) δ 7.86 (d, J=10.0 Hz, 2H), 7.41 (d, J=10.0 Hz, 2H), 7.36-7.33 (m, 2H), 3.88 (t, J=7.5 Hz, 4H), 3.72 (t, J=7.0 Hz, 4H), 3.65-3.60 (m, 20H), 3.36 (t, J=6.5 Hz, 4H); 13C NMR (126 MHz, CDCl3) δ 167.2, 167.0, 160.8, 134.9, 131.4, 127.6, 125.5, 124.1, 113.6, 70.6, 70.5, 70.0, 69.9, 67.8, 50.6, 37.4; [M+H]+ calc'd 711.2733, found 711.2730.

### Azide Intermediate for Compound 119: 5,5-(perfluoropropane-2,2-diyl)bis(2-(2-(2-(2-(2-azidoethoxy)ethoxy)-ethoxy)ethyl)-isoindoline-1,3-dione)

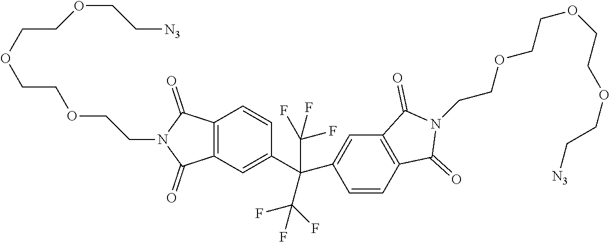 [SMILES: [N-]=[N+]=NCCOCCOCCOCCN1C(=O)c2ccc(C(c3ccc4c(c3)C(=O)N(CCOCCOCCOCCN=[N+]=[N-])C4=O)(C(F)(F)F)C(F)(F)F)cc2C1=O]

Method C; 1H NMR (500 MHz, CDCl3) δ 7.90 (d, J=10.0 Hz, 2H), 7.81 (s, 2H), 7.74 (d, J=10.0 Hz, 2H), 3.91 (t, J=7.0 Hz, 4H), 3.74 (t, J=7.0 Hz, 4H), 3.65-3.60 (m, 20H), 3.36 (t, J=6.0 Hz, 4H); 13C NMR (126 MHz, CDCl3) δ 167.0, 166.8, 138.6, 135.5, 132.9, 132.6, 124.7, 123.5, 70.6, 70.6, 70.0, 69.9, 67.7, 50.6, 37.6; [M+H]+ calc'd 845.2688, found 845.2691.

### Azide Intermediate for Compound 120: 5,5-sulfonylbis(2-(2-(2-(2-(2-azidoethoxy)ethoxy)ethoxy)ethyl)isoindoline-1,3-dione)

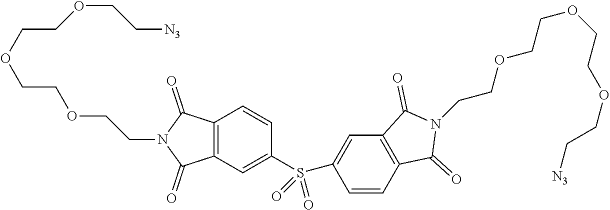 [SMILES: [N-]=[N+]=NCCOCCOCCOCCN1C(=O)c2ccc(S(=O)(=O)c3ccc4c(c3)C(=O)N(CCOCCOCCOCCN=[N+]=[N-])C4=O)cc2C1=O]

Method C; 1H NMR (500 MHz, CDCl3) δ 8.37 (d, J=1.0 Hz, 2H), 8.34 (m, 2H), 8.00 (d, J=8.0 Hz, 2H), 3.89 (t, J=6.0 Hz, 4H), 3.71 (t, J=5.5 Hz, 4H), 3.62-3.54 (m, 20H), 3.34 (t, J=5.0 Hz, 4H); 13C NMR (126 MHz, CDCl3) δ 166.1, 165.9, 146.0, 136.2, 133.6, 133.3, 124.5, 122.8, 70.5, 70.0, 70.0, 67.5, 50.6, 37.8; [M+H]+ calc'd 759.2403, found 759.2407.

### Azide Intermediate for Compound 121: 5,5-carbonylbis(2-(2-(2-(2-(2-azidoethoxy)ethoxy)ethoxy)ethyl)isoindoline-1,3-dione)

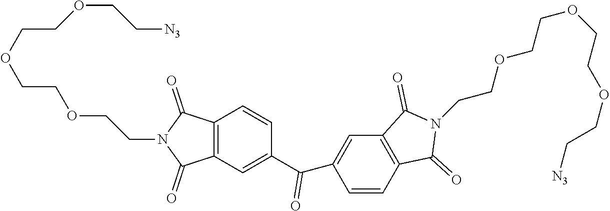 [SMILES: [N-]=[N+]=NCCOCCOCCOCCN1C(=O)c2ccc(C(=O)c3ccc4c(c3)C(=O)N(CCOCCOCCOCCN=[N+]=[N-])C4=O)cc2C1=O]

Method C; 1H NMR (500 MHz, CDCl3) δ 8.14 (m, 4H), 8.00 (d, J=8.0 Hz, 2H), 3.93 (t, J=5.5 Hz, 4H), 3.75 (t, J=6.0 Hz, 4H), 3.65-3.59 (m, 20H), 3.35 (t, J=5.5 Hz, 4H); 13C NMR (126 MHz, CDCl3) δ 193.0, 167.0, 167.0, 145.5, 135.4, 135.4, 132.6, 124.2, 123.7, 70.6, 70.1, 70.0, 67.8, 50.6, 37.7; [M+H]+ calc'd 723.2733, found 723.2732.

### Azide Intermediate for Compound 122: 2,6-bis(2-(2-(2-(2-azidoethoxy)ethoxy)ethoxy)ethyl)pyrrolo[3,4-f]isoindole-1,3,5,7(2H,6H)-tetraone

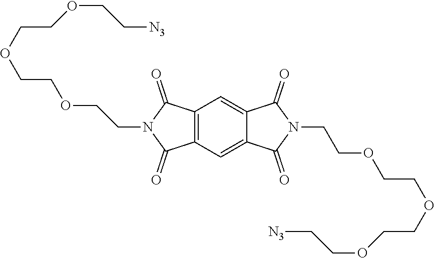 [SMILES: [N-]=[N+]=NCCOCCOCCOCCn1c(=O)c2cc3c(=O)n(CCOCCOCCOCCN=[N+]=[N-])c(=O)c3cc2c1=O]

Method C; 1H NMR (500 MHz, CDCl3) δ 8.23 (s, 2H), 3.93 (t, J=7.0 Hz, 4H), 3.75 (t, J=7.0 Hz, 4H), 3.65-3.59 (m, 20H), 3.34 (t, J=6.5 Hz, 4H); 13C NMR (126 MHz, CDCl3) δ 166.0, 137.2, 118.0, 70.6, 69.9, 69.9, 67.5, 67.8, 50.6, 37.8; [M+H]+ calc'd 619.2471 found 619.2472

### 4-((1-(1-phenyl-5,8,11-trioxa-2-azatridecan-13-yl)-1H-1,2,3-triazol-4-yl)methyl)thiomorpholine 1,1-dioxide

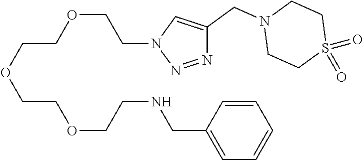 [SMILES: O=S1(=O)CCN(Cc2cn(CCOCCOCCOCCNCc3ccccc3)nn2)CC1]

Method D; 1H NMR (500 MHz, CDCl3) δ 7.66 (s, 1H), 7.34-7.26 (m, 4H), 7.23 (ddd, J=8.6, 5.3, 2.3 Hz, 1H), 4.49 (t, J=4.9 Hz, 2H), 3.83 (t, J=5.0 Hz, 2H), 3.79 (d, J=4.8 Hz, 2H), 3.62-3.56 (m, 10H), 3.05-2.99 (m, 8H), 2.81 (t, J=5.2 Hz, 2H); 13C NMR (126 MHz, CDCl3) δ 143.1, 139.8, 128.4, 128.2, 127.0, 123.9, 70.4, 70.4, 70.4, 70.2, 69.3, 53.7, 52.0, 51.3, 50.4, 50.2, 48.5; [M+H]+ calc'd 482.2432, found 482.2435.

### 4-((1-(12-allyl-3,6,9-trioxa-12-azapentadec-14-en-1-yl)-1H-1,2,3-triazol-4-yl)methyl)thiomorpholine 1,1-dioxide

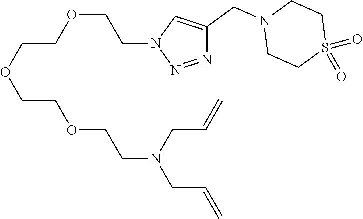 [SMILES: C=CCN(CC=C)CCOCCOCCOCCn1cc(CN2CCS(=O)(=O)CC2)nn1]

Method D; 1H NMR (500 MHz, CDCl3) δ 7.67 (s, 1H), 5.82 (ddt, J=16.8, 10.1, 6.5 Hz, 2H), 5.14 (dt, J=17.1, 1.8 Hz, 2H), 5.10 (ddt, J=10.1, 2.1, 1.1 Hz, 2H), 4.52 (t, J=5.0 Hz, 2H), 3.85 (t, J=5.0 Hz, 2H), 3.80 (s, 2H), 3.61-3.57 (m, 8H), 3.54 (t, J=6.2 Hz, 2H), 3.11 (dt, J=6.5, 1.2 Hz, 4H), 3.06-3.01 (m, 8H), 2.64 (t, J=6.2 Hz, 2H); 13C NMR (126 MHz, CDCl3) δ 143.1, 135.5, 123.9, 117.5, 70.5, 70.4, 70.4, 70.3, 69.7, 69.3, 57.5, 52.4, 52.0, 51.3, 50.4, 50.2; [M+H]+ calc'd 472.2588, found 472.2586.

### N-(2-(2-(2-(2-(4-((1,1-dioxidothiomorpholino)methyl)-JH-1,2,3-triazol-1-yl)ethoxy)ethoxy)ethoxy)ethyl)-2,2,2-trifluoroacetamide

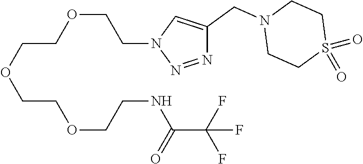 [SMILES: O=C(NCCOCCOCCOCCn1cc(CN2CCS(=O)(=O)CC2)nn1)C(F)(F)F]

Method A; 1H NMR (400 MHz, CDCl3) δ 7.63 (s, 1H), 7.39 (s, 1H), 4.50 (t, J=5.1 Hz, 2H), 3.85 (t, J=5.1 Hz, 2H), 3.80 (s, 2H), 3.63-3.53 (m, 10H), 3.50 (q, J=5.4 Hz, 2H), 3.08 (s, 8H); 13C NMR (101 MHz, CDCl3) δ 157.5 (q, J=37.4 Hz), 143.8, 124.1, 117.4 (td, J=289 Hz), 70.4, 70.4, 70.2, 69.4, 68.7, 52.0, 51.2, 50.4, 50.3, 39.7; 19F NMR (377 MHz, CDCl3) δ −75.7; [M+H]+ calc'd 488.1785, found 488.1780.

### tert-butyl (2-(2-(2-(2-(4-((1,1-dioxidothiomorpholino)methyl)-1H-1,2,3-triazol-1-yl)ethoxy)ethoxy)ethoxy)ethyl)carbamate

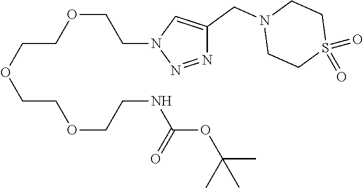 [SMILES: CC(C)(C)OC(=O)NCCOCCOCCOCCn1cc(CN2CCS(=O)(=O)CC2)nn1]

Method A; 1H NMR (400 MHz, CDCl3) δ 7.69 (s, 1H), 5.05 (br s, 1H), 4.55 (t, J=5.1 Hz, 2H), 3.85 (t, J=5.1 Hz, 2H), 3.88 (t, J=5.1 Hz, 2H), 3.82 (s, 2H), 3.60 (m, 8H), 3.53 (t, J=5.1 Hz, 2H), 3.30 (br s, 2H), 3.06 (m, 8H), 1.68 (s, 1H), 1.43 (s, 9H); 13C NMR (101 MHz, CDCl3) δ 155.9, 143.2, 124.0, 79.2, 77.3, 70.5 (2C), 70.4, 70.2, 70.2, 69.4, 53.5, 52.0, 51.4, 50.4, 50.3, 40.3, 28.4; [M+H]+ calc'd 492.2486, found 492.2481.

### (9H-fluoren-9-yl)methyl (2-(2-(2-(2-(4-((1,1-dioxidothiomorpholino)methyl)-1H-1, 2, 3-triazol-1-yl)ethoxy)ethoxy)ethoxy)ethyl)carbamate

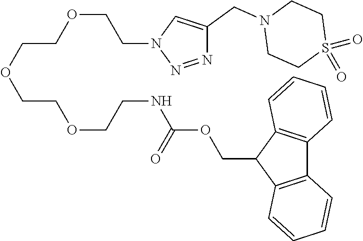 [SMILES: O=C(NCCOCCOCCOCCn1cc(CN2CCS(=O)(=O)CC2)nn1)OCC1c2ccccc2-c2ccccc21]

Method A (Except no base and at RT throughout); 1H NMR (500 MHz, CDCl3) δ 7.75 (d, J=7.5 Hz, 3H), 7.59 (d, J=7.0 Hz, 4H), 7.39 (t, J=7.4 Hz, 3H), 7.30 (t, J=7.4 Hz, 3H), 5.47 (t, J=5.8 Hz, 1H), 4.47 (t, J=5.1 Hz, 2H), 4.41 (d, J=6.8 Hz, 2H), 4.20 (t, J=6.8 Hz, 1H), 3.81 (t, J=5.0 Hz, 2H), 3.76 (s, 2H), 3.60-3.53 (m, 10H), 3.37 (q, J=5.4 Hz, 2H), 3.03-2.98 (m, 8H); 13C NMR (126 MHz, CDCl3) δ 156.44, 143.87, 143.08, 141.24, 127.64, 126.98, 124.95, 123.87, 119.92, 70.39, 70.36, 70.32, 70.16, 69.97, 69.29, 66.37, 51.91, 51.24, 50.33, 50.17, 47.21, 40.85; [M+H]+ calc'd 614.2643, found 614.2644.

### 2,5-bis(2-(2-(2-(2-(4-((1,1-dioxidothiomorpholino)methyl)-1H-1,2,3-triazol-1-yl)ethoxy)ethoxy)ethoxy)ethyl)tetrahydrocyclobuta[1,2-c:3,4-c]dipyrrole-1,3,4,6(2H,5H)-tetraone

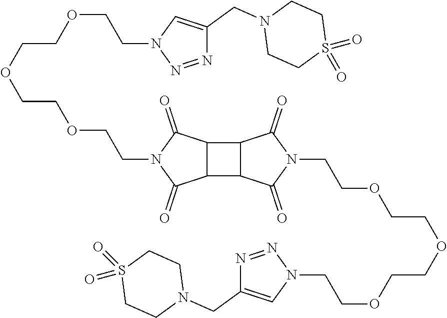 [SMILES: O=C1C2C(C(=O)N1CCOCCOCCOCCn1cc(CN3CCS(=O)(=O)CC3)nn1)C1C(=O)N(CCOCCOCCOCCn3cc(CN4CCS(=O)(=O)CC4)nn3)C(=O)C21]

Method D; 1H NMR (500 MHz, CDCl3) δ 7.71 (s, 2H), 4.56 (t, J=5.0 Hz, 4H), 3.87 (t, J=5.1 Hz, 4H), 3.84 (s, 4H), 3.81 (t, J=5.5 Hz, 4H), 3.70 (t, J=5.5 Hz, 4H), 3.57 (dddd, J=13.9, 8.9, 7.2, 3.6 Hz, 16H), 3.45 (s, 4H), 3.11-3.02 (m, 16H); 13C NMR (126 MHz, CDCl3) δ 175.21, 143.20, 124.06, 70.49, 70.42, 70.32, 69.87, 69.38, 66.57, 52.04, 51.38, 50.42, 50.25, 41.43, 38.55; [M+H]+ calc'd 943.3648, found 943.3655.

### 1-(2-(2-(2-(2-(4-((1,1-dioxidothiomorpholino)methyl)-JH-1,2,3-triazol-1-yl)ethoxy)ethoxy)ethoxy)ethyl)pyrrolidine-2,5-dione

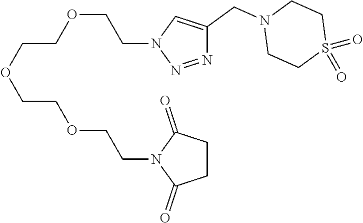 [SMILES: O=C1CCC(=O)N1CCOCCOCCOCCn1cc(CN2CCS(=O)(=O)CC2)nn1]

Method D; 1H NMR (500 MHz, CDCl3) δ 7.66 (s, 1H), 4.50 (t, J=5.0 Hz, 2H), 3.83 (t, J=5.0 Hz, 2H), 3.78 (s, 2H), 3.66 (t, J=5.7 Hz, 2H), 3.59 (t, J=5.7 Hz, 2H), 3.57-3.50 (m, 8H), 3.05-3.01 (m, 4H), 3.01-2.97 (m, 4H), 2.66 (s, 4H); 13C NMR (126 MHz, CDCl3) δ 177.1, 143.1, 123.9, 70.4, 70.3, 69.7, 69.2, 67.0, 51.9, 51.2, 50.3, 50.1, 37.7, 28.0; [M+H]+ calc'd 474.2017, found 474.2010.

### 4-((1-(2-(2-(2-(2-((1,3-dithiolan-2-ylidene)amino)ethoxy)ethoxy)ethoxy)ethyl)-1H-1,2,3-triazol-4-yl)methyl)thiomorpholine 1,1-dioxide

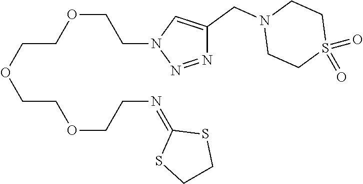 [SMILES: O=S1(=O)CCN(Cc2cn(CCOCCOCCOCCN=C3SCCS3)nn2)CC1]

Method B; 1H NMR (500 MHz, CDCl3) δ 7.71 (s, 1H), 4.52 (t, J=5.0 Hz, 2H), 3.85 (t, J=5.0 Hz, 2H), 3.80 (s, 2H), 3.72 (t, J=5.9 Hz, 2H), 3.65-3.59 (m, 8H), 3.54 (t, J=7.1, 5.2 Hz, 2H), 3.42 (t, J=6.0 Hz, 2H), 3.38 (t, J=7.1, 5.2 Hz, 2H), 3.07-3.03 (m, 4H), 3.03-2.99 (m, 4H); 13C NMR (126 MHz, CDCl3) δ 169.3, 143.1, 124.0, 70.8, 70.4, 69.3, 58.6, 52.0, 51.3, 50.3, 50.2, 37.8, 34.7; [M+H]+ calc'd 494.1560, found 494.1557.

### N-(2-(2-(2-(2-(4-((1,1-dioxidothiomorpholino)methyl)-1H-1,2,3-triazol-1-yl)ethoxy)ethoxy)ethoxy)ethyl)-4-nitrobenzenesulfonamide

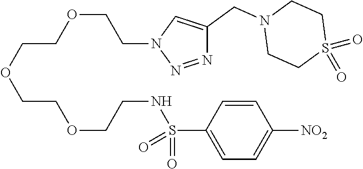 [SMILES: O=[N+]([O-])c1ccc(S(=O)(=O)NCCOCCOCCOCCn2cc(CN3CCS(=O)(=O)CC3)nn2)cc1]

Method A; 1H NMR (500 MHz, CDCl3) δ 8.28 (d, J=8.5 Hz, 2H), 8.02 (d, J=8.4 Hz, 2H), 7.67 (s, 1H), 6.39 (t, J=5.9 Hz, 1H), 4.51 (t, J=5.0 Hz, 2H), 3.85 (t, J=5.1 Hz, 2H), 3.77 (s, 2H), 3.60-3.42 (m, 11H), 3.12 (q, J=5.3 Hz, 2H), 3.05-3.00 (m, 4H), 3.00-2.93 (m, 4H); 13C NMR (126 MHz, CDCl3) δ 149.6, 146.0, 142.9, 128.1, 123.9, 123.7, 70.1, 70.0, 69.9, 69.7, 69.0, 68.9, 51.6, 51.0, 50.1, 49.9, 42.7; [M+H]+ calc'd 577.1745, found 577.1743.

### 2,7-bis(2-(2-(2-(2-(4-((1,1-dioxidothiomorpholino)methyl)-1H-1,2,3-triazol-1-yl)ethoxy)ethoxy)ethoxy)ethyl)benzo[lmn][3,8]phenanthroline-1,3,6,8(2H, 7H)-tetraone

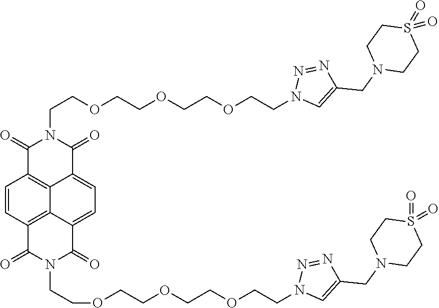 [SMILES: O=C1c2ccc3c4c(ccc(c24)C(=O)N1CCOCCOCCOCCn1cc(CN2CCS(=O)(=O)CC2)nn1)C(=O)N(CCOCCOCCOCCn1cc(CN2CCS(=O)(=O)CC2)nn1)C3=O]

Method D; 1H NMR (500 MHz, CDCl3) δ 8.69 (s, 4H), 7.68 (s, 2H), 4.51 (t, J=4.5 Hz, 4H), 4.43 (t, J=6.0 Hz, 4H), 3.84-3.81 (m, 12H), 3.69-3.67 (m, 4H), 3.59-3.54 (m, 12H), 3.06-3.02 (m, 16H); 13C NMR (126 MHz, CDCl3) δ 162.8, 143.1, 131.0, 126.7, 126.5, 124.0, 70.5, 70.4, 70.0, 69.3, 67.7, 52.0, 51.3, 50.4, 50.2, 39.5; [M+H]+ calc'd 1015.3648, found 1015.3633.

### 5,6,12,13-tetrachloro-2,9-bis(2-(2-(2-(2-(4-((1,1-dioxidothiomorpholino)methyl)-1H-1,2,3-triazol-1-yl)ethoxy)ethoxy)ethoxy)ethyl)anthra[2,1,9-def:6,5,10-def]diisoquinoline-1,3,8,10(2H,9H)-tetraone

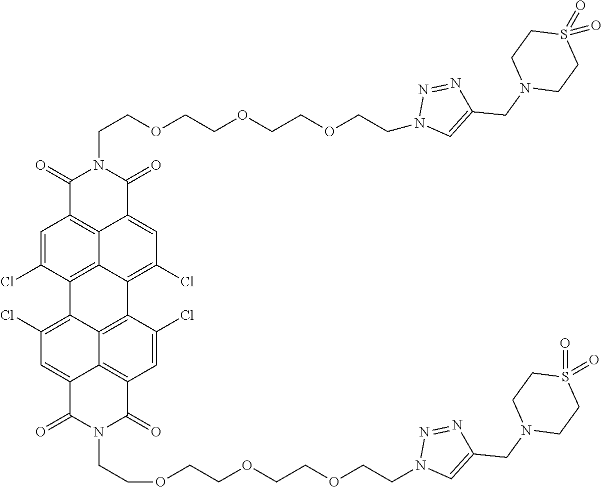 [SMILES: O=C1c2cc(Cl)c3c4c(Cl)cc5c6c(cc(Cl)c(c7c(Cl)cc(c2c37)C(=O)N1CCOCCOCCOCCn1cc(CN2CCS(=O)(=O)CC2)nn1)c64)C(=O)N(CCOCCOCCOCCn1cc(CN2CCS(=O)(=O)CC2)nn1)C5=O]

Method D; 1H NMR (500 MHz, CDCl3) δ 8.69 (s, 4H), 7.68 (s, 2H), 4.51 (t, J=5.0 Hz, 4H), 3.69-3.67 (m, 4H), 3.85-3.81 (m, 12H), 3.70-3.67 (m, 4H), 3.60-3.55 (m, 12H), 3.06-3.01 (m, 16H); 13C NMR (126 MHz, CDCl3) δ 162.2, 143.1, 135.3, 132.9, 131.4, 128.6, 123.9, 123.3, 123.0, 70.5, 70.4, 70.1, 69.3, 67.7, 53.4, 52.0, 51.3, 50.4, 50.2, 39.6; [M+H]+ calc'd 1275.2402, found 1275.2389.

### Example 2: Synthesis of Intermediates of Exemplary Compounds of Formula (I), (II), and (III)

### Step 1:

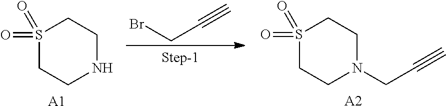

1.60 kg of A1 was dissolved in 10 vol acetone in a 50 L jacketed reactor. K₂CO₃ (2 equiv) was added, and the mixture was cooled down to 0-5° C., followed by dropwise addition of propargyl bromide (1.1 equiv). The mixture was gradually warmed up to 25° C. and agitated for 16 h. After the reaction was complete as shown by HPLC, the mixture was filtered through Magnesol, followed by rinsing with EtOAc. The cake was solvent exchanged EtOAc (3×5 vol), then the mixture was purified by HPLC (99.6%) to yield A2.

### Step 2:

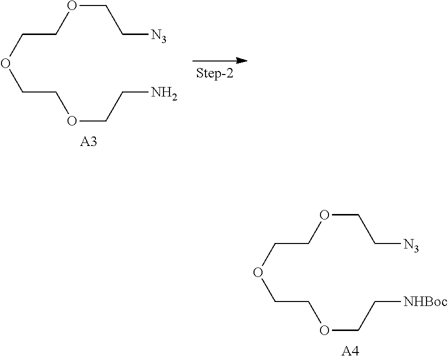

A3 (1.49 kg) was placed in a 50 L jacketed reactor with dichloromethane (3 vol) and deionized water (11 vol), and mixed to yield a turbid solution. The mixture was cooled to 5° C., followed by addition of Na₂CO₃ (2 equiv) and Boc₂O (1 equiv), which was added over a period of 30 min. The resulting mixture was warmed to room temperature overnight to allow the reaction to come to completion as observed by HPLC. Two layers of the mixture were separated, and the aqueous layer was back-extracted with dichloromethane (2×3 vol). The combined the organic layers were dried over Na₂SO₄, filtered, then solvent exchanged into THE prior to HPLC purification (87%) to yield A4.

### Step 3:

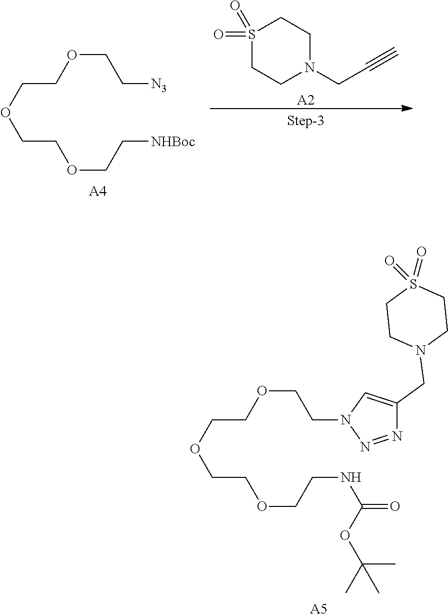

A4 (100 g) was added to a 2 L jacketed flask, followed by addition of THE (4 vol) to obtain a slurry. CuSO₄·5H₂O (0.01 equiv) in 0.5 vol of deionized water was added, followed by sodium L-ascorbate (0.5 vol) in deionized water. The mixture was heated the mixture to 55° C., then a solution of A2 in THE was slowly added over a period of 1.5 h and stirred at temperature for 1 h. The transformation was deemed complete at this stage, as indicated by HPLC analysis. The mixture was then cooled down to room temperature, followed by addition of dichloromethane (10 vol) and 10 vol of 1:1 sat′d NH₄Cl:28-30% NH₄OH. The layers were separated after agitation, and the organic layer was washed with 2×10 vol of 1:1 sat′d NH4Cl:28-30% NH₄OH. The organic layer was dried over Na₂SO₄, filtered, washed with dichloromethane, then concentrated down to dryness to obtain an oil. The crude product was purified by silica gel chromatography and purified to obtain an oil of A5 (98.1%).

### Step 4

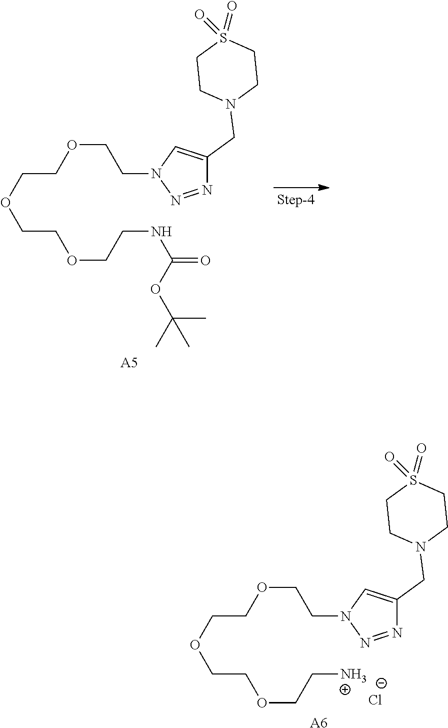

A5 (210 g) was combined with 5 vol of water, then concentrated under vacuum at ≤70° C. to achieve a target 4 vol solution. 6 M aq. HCl (10 equiv) was added, and the temperature was maintained at ≤30° C. The resulting mixture was stirred at 20±5° C. for 1 h, then HPLC analysis showed the reaction was complete. The reaction mixture was concentrated to yield a solution (97.1% purity).

### Step 5

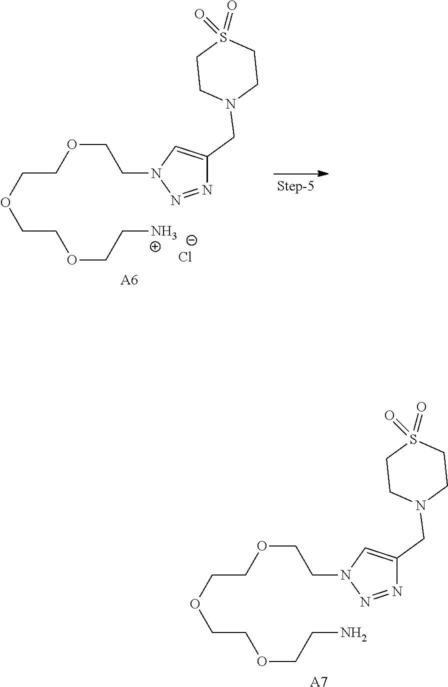

2 vol of 6 M aq. NaOH was added to an aqueous solution of A6 at ≤30° C. The pH of the mixture was adjusted to pH=11 with an additional volume of 6 M aq. NaOH, and the mixture was concentrated. 5 vol of MeCN was added, and the mixture was filtered and the filter cake was with 0.5 vol of MeCN. The solvent exchange was repeated 3 times to yield the final product A7 (96.4% purity).

### Example 3: Synthesis of Exemplary Compounds of Formula (I)

Exemplary compounds of Formula (I) were synthesized as outlined in Scheme 1 below.

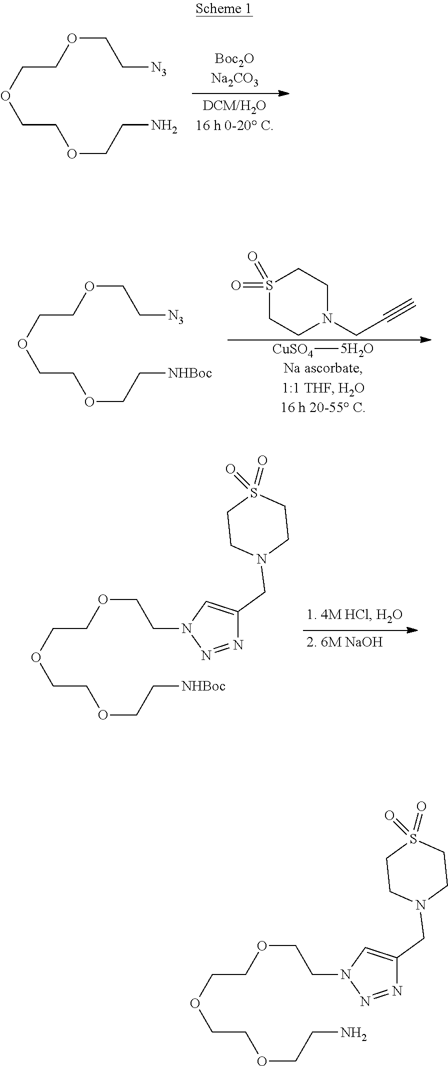

As shown, 2-(2-(2-(2-azidoethoxy)ethoxy)ethoxy)ethan-1-amine is incubated with Boc₂O and Na₂CO₃ in a mixture of dichloromethane and water. Following purification of the Boc-protected amine, the azide is coupled to an exemplary alkyne in the presence of CuSO₄ and sodium ascorbate to yield the compound of Formula (I). The amine-protecting group may then be deprotected to afford the free amine.

### Example 4: Synthesis of Exemplary Compounds of Formula (I)

Exemplary compounds of Formula (I) were synthesized as outlined in Scheme 2 below

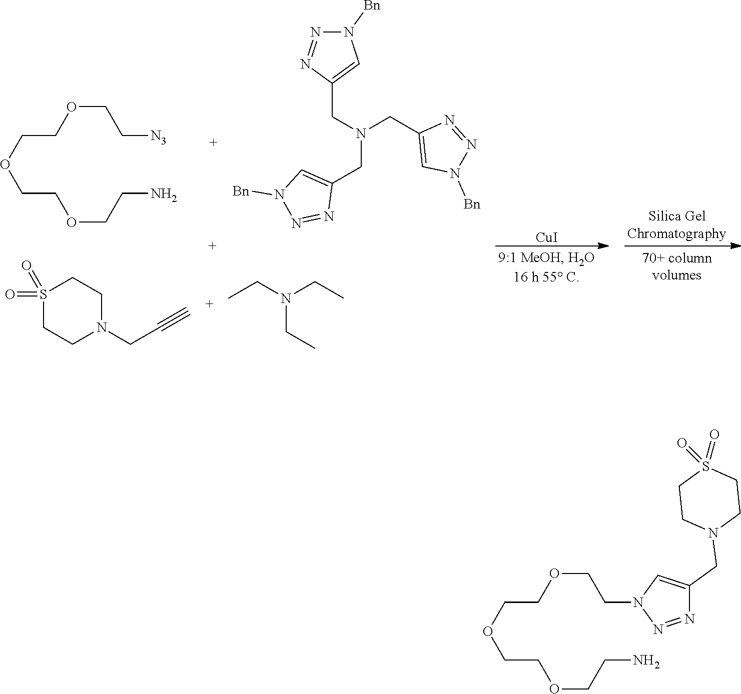

### Example 5: Synthesis of Exemplary Compounds of Formula (I)

Exemplary compounds of Formula (I) were synthesized as outlined in Scheme 3 below

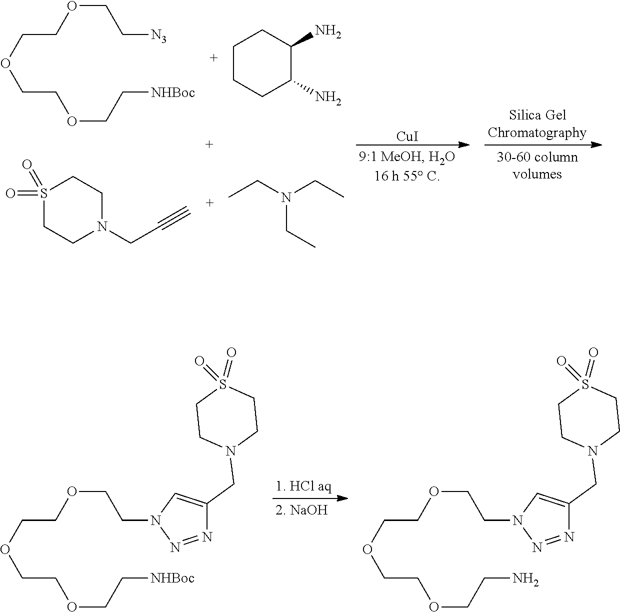

### Example 6: Synthesis of Exemplary Compounds of Formula (I)

Exemplary compounds of Formula (I) were synthesized as outlined in Scheme 4 below

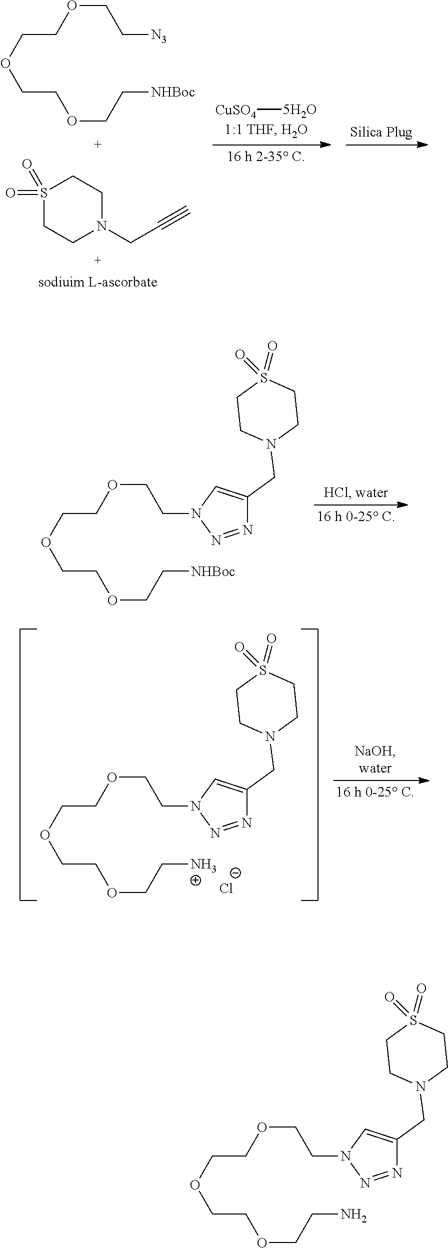

### Example 7: Synthesis of Exemplary Compounds of Formula (I)

Exemplary compounds of Formula (I) were synthesized as outlined in Scheme 5 below

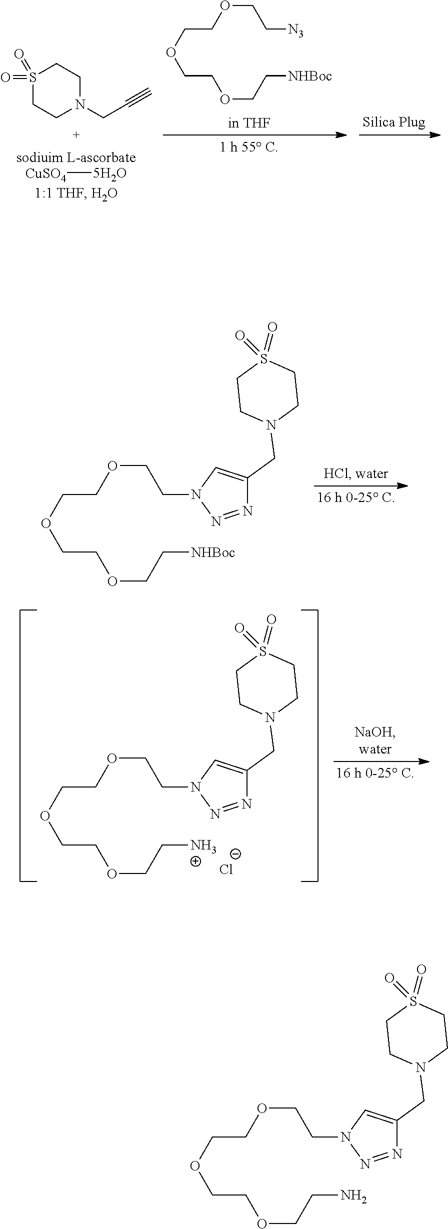

### Example 8: Determination of Onset Temperatures for Exemplary Compounds of Formula (I), (II), and (III)

Exemplary compounds of Formula (I) and (II) were analyzed using differential scanning calorimetry (DSC) to determine the relevant onset temperature (° C.). The DSC tests were completed using a DSC 214 Polyma-Differential Scanning Calorimeter manufactured by Netzsch™ Group of Selb, Germany. DSC tests measure, in part, the temperature response of a sample as it is steadily heated. Before the study commences, verification of the instrument's calibration is performed, based on the manufacturer's protocols. Testing was conducted according to the ASTM Standard E537-02, Standard Test Method for The Thermal Stability of Chemicals by Differential Scanning Calorimetry. Sample preparation for the DSC tests was conducted as follows:
1. Test samples were transferred into a high-pressure stainless-steel test crucible in a glove bag under an ultrahigh purity nitrogen atmosphere
2. Samples were tested in the temperature range of 20° C. to 490° C. in a closed test cell with a ramp heating rate of 10° C./min
3. Tests were conducted under a constant flow of nitrogen
   After testing of sample concluded, the sample crucible was weighed sealed to obtain the final weight of the sample. The sample crucible was then weighted again, after the gold seal of the stainless-steel crucible lid is puncture under a chemical hood, allowing the potential non-condensable gases to escape. The calculation of the difference between sealed crucible vs the pierced crucible is the amount of non-condensable gas produced by the sample. A summary of the onset temperature and the maximum exothermal output derived from these DSC analyses are described in Table 3.

TABLE 3
Summary of energetics data for exemplary compounds of
Formula (I) and (II)

| | 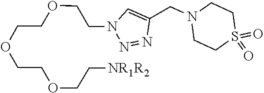 | | | | |
|---|---|---|---|---|---|
| | (C + X + | | | Max | Max |
| R₁ =; | O + | | Onset T | Exo | Exo |
| R₂ = | S)/N | Phase | (° C.) | (J/g) | (kJ/mol) |
| —H; —H | 4.2 | Liquid | 315 | 1243 | 487 |
| —H; | 5.6 | Liquid | 297 | 1058 | 510 |
| —CH₂Ph | | | | | |
| R₁, R₂ = | 5.4 | Liquid | 327 | 596 | 281 |
| —CH₂CHCH₂ | | | | | |
| H; —COCF₃ | 5.4 | Liquid | 294 | 969 | 472 |
| H; Boc | 5.6 | Liquid | 327 | 645 | 317 |
| H; Fmoc | 7.6 | Solid | 139 | 1040 | 642 |
| H; Nosyl | 5.3 | Liquid | 294 | 1815 | 1047 |
| 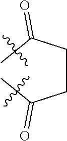 [SMILES: *C(=O)CCC(*)=O] | 5.4 | Liquid | 294 | 540 | 256 |
| 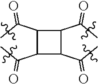 [SMILES: *C(=O)C1C(C(*)=O)C(C(*)=O)C1C(*)=O] | 5.4 | Solid | 290 | 1037 518* | 977 488* |
| 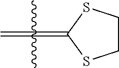 [SMILES: *=C1SCCS1] | 5.3 | Liquid | 264 | 885 | 437 |

*The values in with the asterisk represent the effective energy for 1 equivalent.

1. A compound of Formula (I): 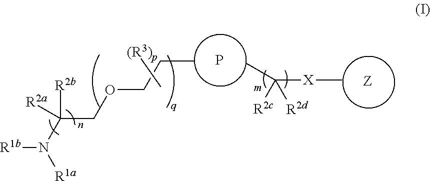
or a pharmaceutically acceptable salt thereof, wherein:
Ring P is heterocyclyl or heteroaryl, each of which is optionally substituted with 1-6 R⁴;
Ring Z is cycloalkyl, heterocyclyl, aryl, or heteroaryl, each of which is optionally substituted with 1-6 R⁵;
X is O, S, N(R^{A}), C₁-C₁₂ alkylene, C₁-C₁₂ alkenylene, C₂-C₁₂ heteroalkylene, or absent;
R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group;
R^{1b} is an amine-protecting group; or
R^{1a} and R^{1b} may be taken together to form a cycloalkyl, heterocyclyl, aryl, or heteroaryl ring, each of which is optionally substituted with 1-6 R⁶;
each of R^{2a}, R^{2b}, R^{2c}, and R^{2d} is independently hydrogen, C₁-C₁₂ alkyl, C₂-C₁₂ heteroalkyl, halogen, or OR^{C}; or
R^{2a} and R^{2b} or R^{2c} and R^{2d} are taken together to form an oxo;
each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
each R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
m and n are each independently 0, 1, 2, 3, 4, 5, or 6;
p is 0, 1, 2, 3, 4, or 5; and
q is an integer from 0 to 25.

2. The compound of claim 1, wherein Ring P is a heteroaryl.

3. The compound of claim 1, wherein Ring P is a five-membered or six-membered nitrogen heteroaryl.

4. The compound of claim 1, wherein Ring P is triazolyl (e.g., 1,2,3-triazolyl or 1,2,4-triazolyl).

5. The compound of claim 1, wherein Ring P is selected from

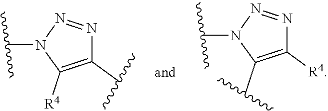

6. The compound of claim 1, wherein Ring Z is a four-membered ring, a five-membered ring, or a six-membered ring (e.g., a six-membered ring).

7. The compound of claim 1, wherein Ring Z is a nitrogen-containing six-membered ring.

8. The compound of claim 1, wherein R^{1a} is hydrogen.

9. The compound of claim 1, wherein R^{1b} comprises a C₁-C₁₂ alkylene, C₂-C₁₂ alkenylene, C₂-C₁₂ heteroalkylene, C₁-C₁₂ haloalkylene, cycloalkyl, heterocyclyl, aryl, or heteroaryl.

10. The compound of claim 1, wherein R^{1b} comprises an acid-labile amine-protecting group or a base-labile amine-protecting group.

11. The compound of claim 1, wherein R^{1b} is selected from 9-fluorenylmethoxycarbonyl (Fmoc), benzyl (Bn), benzoyl (Bz), allyloxycarbonyl (Alloc), 2-(4-nitropheylsulfonyl)ethoxycarbonyl (Nsc), 1,1-dioxobenzo[b]thiophene-2-ylmethyloxycarbonyl (Bsmoc), 1,1-dioxonaphtho[1,2-b]thiophene (Nsmoc), 1-(4,4-dimethyl-2,6-dioxocyclohex-1-ylidene)-3-methylbutyl (ivDde), 2,2,2-trichloroethyloxycarbonyl (Troc), 2-[phenyl(methyl)sulfonio]ethyloxycarbonyl tetrafluoroborate (Pms), tert-butyloxycarbonyl (Boc), p-methoxybenzyl (PMB), carbobenzyloxy (Cbz), acetyl (Ac), trifluoroacetyl, tosyl (Ts), trityl (Trt), 2-(4-biphenyl)-isopropoxycarbonyl (Bpoc), allyl (Al), 2,2,5,5-tetramethyl-1,2,5-azadisilolidine, 1,3-dithiolan-2-imine, nitrobenzenesulfonyl (Nosyl), succinimidyl, and pyromellitic diimide.

12. The compound of claim 1, wherein R^{1b} is selected from tert-butyloxycarbonyl (Boc), 9-fluorenylmethoxycarbonyl (Fmoc), benzyl (Bn), allyl (Al), nitrobenzenesulfonyl (Nosyl), dithiolan-2-imine, and trifluoroacetyl.

13. The compound of claim 1, wherein R^{1b} is tert-butyloxycarbonyl (Boc).

14. The compound of claim 1, wherein R^{1a} and R^{1b} are taken together to form a cycloalkyl, heterocyclyl, aryl, or heteroaryl ring.

15. The compound of claim 14, wherein R^{1a} and R^{1b} are taken together to form a succinimidyl ring.

16. The compound of claim 1, wherein R^{1a} is hydrogen.

17. The compound of claim 1, wherein R^{1a} is hydrogen and R^{1b} is 9-fluorenylmethoxycarbonyl (Fmoc), benzyl (Bn), benzoyl (Bz), allyloxycarbonyl (Alloc), 2-(4-nitropheylsulfonyl)ethoxycarbonyl (Nsc), 1,1-dioxobenzo[b]thiophene-2-ylmethyloxycarbonyl (Bsmoc), 1,1-dioxonaphtho[1,2-b]thiophene (Nsmoc), 1-(4,4-dimethyl-2,6-dioxocyclohex-1-ylidene)-3-methylbutyl (ivDde), 2,2,2-trichloroethyloxycarbonyl (Troc), 2-[phenyl(methyl)sulfonio]ethyloxycarbonyl tetrafluoroborate (Pms), tert-butyloxycarbonyl (Boc), p-methoxybenzyl (PMB), carbobenzyloxy (Cbz), acetyl (Ac), trifluoroacetyl, tosyl (Ts), trityl (Trt), 2-(4-biphenyl)-isopropoxycarbonyl (Bpoc), allyl (Al), 2,2,5,5-tetramethyl-1,2,5-azadisilolidine, 1,3-dithiolan-2-imine, nitrobenzenesulfonyl (Nosyl), succinimidyl, and pyromellitic diimide.

18. The compound of claim 1, wherein R^{1a} is hydrogen and R^{1b} is tert-butyloxycarbonyl (Boc).

19. The compound of claim 1, wherein each of R^{2a}, R^{2b}, R^{2c}, and R^{2d} is independently hydrogen.

20. The compound of claim 1, wherein each of m and n is 1 or 2.

21. The compound of claim 1, wherein q is 1, 2, 3, 4, 5, or 6.

22. The compound of claim 1, wherein p is 0.

23. The compound of claim 1, wherein the compound of Formula (I) is a compound of Formula (I-a): 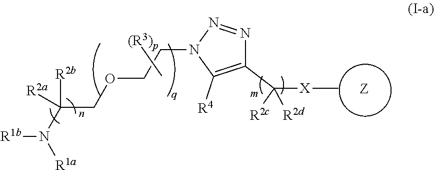
or a pharmaceutically acceptable salt thereof, wherein:
Ring Z is cycloalkyl, heterocyclyl, aryl, or heteroaryl, each of which is optionally substituted with 1-6 R⁵;
X is O, S, N(R^{A}), C₁-C₁₂ alkylene, C₁-C₁₂ alkenylene, C₂-C₁₂ heteroalkylene, or absent;
R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group;
R^{1b} is an amine-protecting group; or
R^{1a} and R^{1b} may be taken together to form a cycloalkyl, heterocyclyl, aryl, or heteroaryl ring, each of which is optionally substituted with 1-6 R⁶;
each of R^{2a}, R^{2b}, R^{2c}, and R^{2d} is independently hydrogen, C₁-C₁₂ alkyl, C₂-C₁₂ heteroalkyl, halogen, or OR^{C}; or
R^{2a} and R^{2b} or R^{2c} and R^{2d} are taken together to form an oxo;
each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
m and n are each independently 0, 1, 2, 3, 4, 5, or 6;
p is 0, 1, 2, 3, 4, or 5; and
q is an integer from 0 to 25.

24. The compound of claim 1, wherein the compound of Formula (I) is a compound of Formula (I-b): 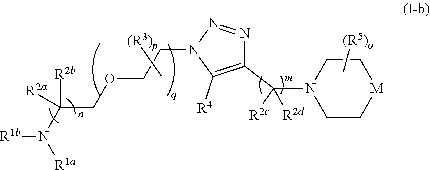
or a pharmaceutically acceptable salt thereof, wherein:
M is C(R′)(R″), N(R′), or S(O)ₓ;
each of R′ and R″ is independently hydrogen, C₁-C₁₂ alkyl, C₂-C₁₂ heteroalkyl, C₁-C₁₂ haloalkyl or halogen; or
each of R′ and R″ is taken together to form an oxo;
R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group;
R^{1b} is an amine-protecting group; or
R^{1a} and R^{1b} may be taken together to form a cycloalkyl, heterocyclyl, aryl, or heteroaryl ring, each of which is optionally substituted with 1-6 R⁶;
each of R^{2a}, R^{2b}, R^{2c}, and R^{2d} is independently hydrogen, C₁-C₁₂ alkyl, C₂-C₁₂ heteroalkyl, or halogen; or
R^{2a} and R^{2b} or R^{2c} and R^{2d} are taken together to form an oxo;
each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
m and n are each independently 0, 1, 2, 3, 4, 5, or 6;
o and p are each independently 0, 1, 2, 3, 4, or 5;
q is an integer from 0 to 25; and
x is 0, 1, or 2.

25. The compound of claim 1, wherein the compound of Formula (I) is a compound of Formula (I-c): 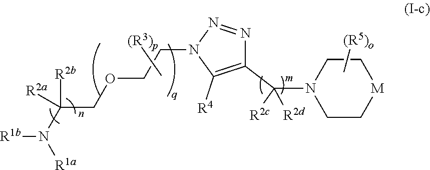
or a pharmaceutically acceptable salt thereof, wherein:
M is C(R′)(R″), N(R′), or S(O)ₓ;
each of R′ and R″ is independently hydrogen, C₁-C₁₂ alkyl, C₂-C₁₂ heteroalkyl, C₁-C₁₂ haloalkyl or halogen; or
each of R′ and R″ is taken together to form an oxo;
R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group;
R^{1b} is an amine-protecting group; or
R^{1a} and R^{1b} may be taken together to form a cycloalkyl, heterocyclyl, aryl, or heteroaryl ring, each of which is optionally substituted with 1-6 R⁶;
each of R^{2a}, R^{2b}, R^{2c}, and R^{2d} is independently hydrogen, C₁-C₁₂ alkyl, C₂-C₁₂ heteroalkyl, or halogen; or
R^{2a} and R^{2b} or R^{2c} and R^{2d} are taken together to form an oxo;
each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
m and n are each independently 0, 1, 2, 3, 4, 5, or 6;
o and p are each independently 0, 1, 2, 3, 4, or 5;
q is an integer from 0 to 25; and
x is 0, 1, or 2.

26. The compound of claim 1, wherein the compound of Formula (I) is a compound of Formula (I-d): 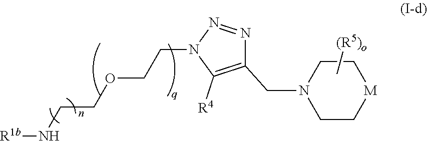
or a pharmaceutically acceptable salt thereof, wherein:
M is C(R′)(R″), N(R′), or S(O)ₓ;
each of R′ and R″ is independently hydrogen, C₁-C₁₂ alkyl, C₂-C₁₂ heteroalkyl, C₁-C₁₂ haloalkyl or halogen; or
each of R′ and R″ is taken together to form an oxo;
R^{1b} is an amine-protecting group;
each R⁵ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}, or N(R^{A})(R^{B});
each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
n is 0, 1, 2, 3, 4, 5, or 6;
o is 0, 1, 2, 3, 4, or 5;
q is an integer from 0 to 25; and
x is 0, 1, or 2.

27. The compound of claim 1, wherein the compound of Formula (I) is a compound of Formula (I-e): 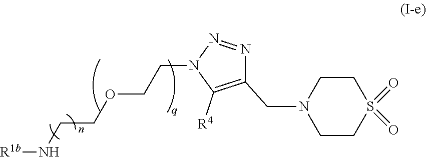
or a pharmaceutically acceptable salt thereof, wherein:
R^{1b} is an amine-protecting group;
R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
n is 0, 1, 2, 3, 4, 5, or 6; and
q is an integer from 0 to 25.

28. The compound of claim 1, wherein the compound is a compound of Formula (I-f): 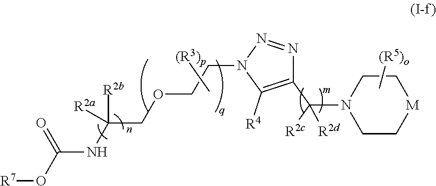
or a pharmaceutically acceptable salt thereof, wherein:
M is C(R′)(R″), N(R′), or S(O)ₓ;
each of R′ and R″ is independently hydrogen, C₁-C₁₂ alkyl, C₂-C₁₂ heteroalkyl, C₁-C₁₂ haloalkyl, or halogen; or
each of R′ and R″ is taken together to form an oxo;
R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
each R⁵ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
R⁷ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, cycloalkyl, heterocyclyl, aryl, or heteroaryl, wherein alkyl, alkenyl, alkynyl, heteroalkyl, haloalkyl, cycloalkyl, heterocyclyl, aryl, and heteroaryl is optionally substituted with 1-6 R⁵;
each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
m and n are each independently 0, 1, 2, 3, 4, 5, or 6;
o and p are each independently 0, 1, 2, 3, 4, or 5;
q is an integer from 0 to 25; and
x is 0, 1, or 2.

29. The compound of claim 1, wherein the compound of Formula (I) is a compound of Formula (I-g): 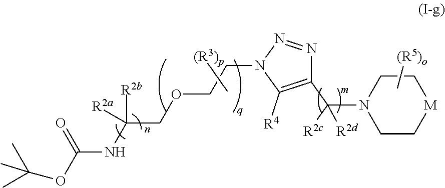
or a pharmaceutically acceptable salt thereof, wherein:
M is C(R′)(R″), N(R′), or S(O)ₓ;
each of R′ and R″ is independently hydrogen, C₁-C₁₂ alkyl, C₂-C₁₂ heteroalkyl, C₁-C₁₂ haloalkyl, or halogen; or
each of R′ and R″ is taken together to form an oxo;
X is O, S, N(R^{A}), C₁-C₁₂ alkylene, C₁-C₁₂ alkenylene, C₂-C₁₂ heteroalkylene, or absent;
each of R^{2a}, R^{2b}, R^{2c}, and R^{2d} is independently hydrogen, C₁-C₁₂ alkyl, C₂-C₁₂ heteroalkyl, or halogen; or
each of R^{2a}, R^{2b}, R^{2c}, and R^{2d} is independently hydrogen, C₁-C₁₂ alkyl, C₂-C₁₂ heteroalkyl, or halogen; or
R^{2a} and R^{2b} or R^{2c} and R^{2d} are taken together to form an oxo;
each of R³ and R⁵ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
each R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
m and n are each independently 0, 1, 2, 3, 4, 5, or 6;
o and p are each independently 0, 1, 2, 3, 4, or 5;
q is an integer from 0 to 25; and
x is 0, 1, or 2.

30. The compound of claim 1, wherein the compound is selected from a compound shown in Table 1 or a pharmaceutically acceptable salt thereof.

31. The compound of claim 1, wherein the onset temperature of the compound is greater than about 150° C. (e.g., greater than about 175° C., 200° C., 225° C., 250° C., 275° C., 300° C., or 325° C.).

32. The compound of claim 1, wherein the onset temperature of the compound is between 150° C. and 350° C. (e.g., between 200° C. and 350° C. or between 250° C. and 350° C.).

33. The compound of claim 1, wherein the maximum exothermal output of the compound is lower than about 750 J/g (e.g., lower than about 700 J/g, 650 J/g, 600 J/g, 550 J/g, 500 J/g, or 450 J/g).

34. The compound of claim 1, wherein the maximum exothermal output of the compound is between about 100 J/g and 700 J/g (e.g., 200 J/g and 600 J/g or between 250 J/g and 550 J/g).

35. The compound of claim 1, wherein the maximum exothermal output of the compound is lower than about 300 kJ/mol, e.g., lower than about 250 kJ/mol, 225 kJ/mol, 200 kJ/mol, and 700 J/g (e.g., 200 J/g and 600 J/g or between 250 J/g and 550 J/g).

36. The compound of claim 1, wherein the compound is a liquid.

37. A composition comprising a compound of any one of claims 1-36.

38. A container comprising greater that about 5 grams, 10 grams, 25 grams, 50 grams, 75 grams, 100 grams, 250 grams, 500 grams, 750 grams, 1 kilogram, 2.5 kilograms, 5 kilograms, 10 kilograms, 20 kilograms, or more of a compound of Formula (I), (II), or (III) or a pharmaceutically acceptable salt thereof according to claim 1, or a composition thereof according to claim 37.

39. The container of claim 38, wherein the container is a vial.

40. A preparation comprising greater that about 5 grams, 10 grams, 25 grams, 50 grams, 75 grams, 100 grams, 250 grams, 500 grams, 750 grams, 1 kilogram, 2.5 kilograms, 5 kilograms, 10 kilograms, 20 kilograms, or more of a compound of Formula (I) or a pharmaceutically acceptable salt thereof according to claim 1, or a composition thereof according to claim 37.

41. The preparation of claim 40, wherein the preparation is substantially pure.

42. The compound of claim 1, wherein the preparation comprises less than 25%, 20%, 15%, 12.5%, 10%. 9%, 8%, 7%, 6%, 5%, 4%, 3%, 2%, 1%, 0.5% of a second compound (e.g., an intermediate in the method of making the compound of Formula (I))

43. A method of preparing a compound of Formula (I): 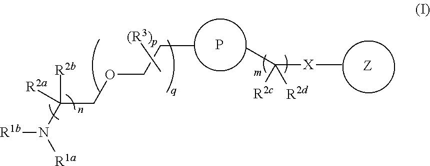
or a pharmaceutically acceptable salt thereof, wherein:
Ring P is heterocyclyl or heteroaryl, each of which is optionally substituted with 1-6 R⁴;
Ring Z is cycloalkyl, heterocyclyl, aryl, or heteroaryl, each of which is optionally substituted with 1-6 R⁵;
X is O, S, N(R^{A}), C₁-C₁₂ alkylene, C₁-C₁₂ alkenylene, C₂-C₁₂ heteroalkylene, or absent;
R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group;
R^{1b} is an amine-protecting group; or
R^{1a} and R^{1b} may be taken together to form a cycloalkyl, heterocyclyl, aryl, or heteroaryl ring, each of which is optionally substituted with 1-6 R⁶;
each of R^{2a}, R^{2b}, R^{2c}, and R^{2d} is independently hydrogen, C₁-C₁₂ alkyl, C₂-C₁₂ heteroalkyl, halogen, or OR^{C}; or
R^{2a} and R^{2b} or R^{2c} and R^{2d} are taken together to form an oxo;
each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
each R⁴ is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
each of R^{A}, R^{B}, R^{C}, and R^{D} is independently hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, cycloalkyl, or heterocyclyl;
m and n are each independently 0, 1, 2, 3, 4, 5, or 6;
p is 0, 1, 2, 3, 4, or 5; and
q is an integer from 0 to 25;
wherein the method comprises reacting a compound of Formula (V): 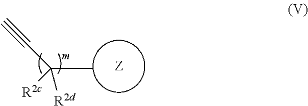
with a compound of Formula (VI): 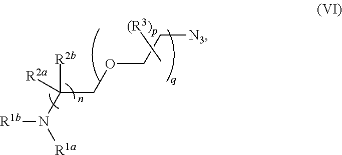
thereby preparing a compound of Formula (I).

44. The compound of claim 43, wherein the compound of Formula (I) is a compound of Formula (I-c): 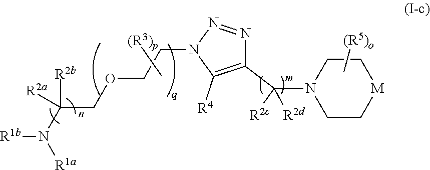
or a pharmaceutically acceptable salt thereof, wherein:
M is C(R′)(R″), N(R′), or S(O)ₓ;
each of R′ and R″ is independently hydrogen, C₁-C₁₂ alkyl, C₂-C₁₂ heteroalkyl, C₁-C₁₂ haloalkyl or halogen; or
each of R′ and R″ is taken together to form an oxo;
R^{1a} is hydrogen, C₁-C₁₂ alkyl, C₂-C₁₂ alkenyl, C₂-C₁₂ alkynyl, C₂-C₁₂ heteroalkyl, C₁-C₁₂ haloalkyl, or an amine-protecting group;
R^{1b} is an amine-protecting group; or
R^{1a} and R^{1b} may be taken together to form a cycloalkyl, heterocyclyl, aryl, or heteroaryl ring, each of which is optionally substituted with 1-6 R⁶;
each of R^{2a}, R^{2b}, R^{2c}, and R^{2d} is independently hydrogen, C₁-C₁₂ alkyl, C₂-C₁₂ heteroalkyl, or halogen; or
R^{2a} and R^{2b} or R^{2c} and R^{2d} are taken together to form an oxo;
each of R³, R⁵, and R⁶ is independently C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
R⁴ is hydrogen, C₁-C₁₂ alkyl, C₂-C₁₂ alkenyl, C₂-C₁₂ alkynyl, C₂-C₁₂ heteroalkyl, C₁-C₁₂ haloalkyl, halogen, oxo, OR^{C}, C(O)OR^{C}, C(O)R^{D}, C(O)N(R^{A}), N(R^{A})C(O)R^{C}; or N(R^{A})(R^{B});
each of R^{A}, R^{B}, R^{C}, and R^{D} is independently hydrogen, C₁-C₁₂ alkyl, C₂-C₁₂ alkenyl, C₂-C₁₂alkynyl, C₂-C₁₂ heteroalkyl, C₁-C₁₂ haloalkyl, halogen, cycloalkyl, or heterocyclyl;
m and n are each independently 0, 1, 2, 3, 4, 5, or 6;
o and p are each independently 0, 1, 2, 3, 4, or 5;
q is an integer from 0 to 25; and
x is 0, 1, or 2;
wherein the method comprises reacting a compound of Formula (V-a): 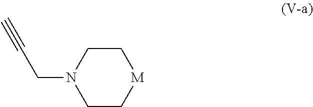
with a compound of Formula (VI-a): 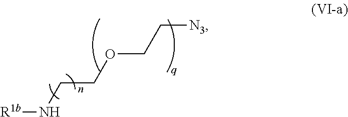
thereby preparing a compound of Formula (I).

45. A preparation of a compound of Formula (I), (II), or (III) described herein comprising a second compound in an amount less than about 25%, 20%, 15%, 12.5%, 10%. 9%, 8%, 7%, 6%, 5%, 4%, 3%, 2%, 1%, 0.75%, 0.5%, 0.25%, 0.1%, or 0.05% (w/w, w/v, v/v, or % by dry weight) of the preparation.

46. The preparation of claim 45, wherein the second compound is present in an amount less than 1% (w/w, w/v, v/v, or % by dry weight) of the preparation.

47. The preparation of claim 45, wherein the second compound is present in an amount less than 0.75% (w/w, w/v, v/v, or % by dry weight) of the preparation.

48. The preparation of claim 45, wherein the second compound is present in an amount less than 0.5% (w/w, w/v, v/v, or % by dry weight) of the preparation.

49. The preparation of claim 45, wherein the second compound is selected from a compound in Table 2.

50. A composition of a compound of Formula (I), (II), or (III) described herein comprising a second compound in an amount less than about 25%, 20%, 15%, 12.5%, 10%. 9%, 8%, 7%, 6%, 5%, 4%, 3%, 2%, 1%, 0.75%, 0.5%, 0.25%, 0.1%, or 0.05% (w/w, w/v, v/v, or % by dry weight) of the composition.

51. The composition of claim 50, wherein the second compound is present in an amount less than 1% (w/w, w/v, v/v, or % by dry weight) of the composition.

52. The composition of claim 50, wherein the second compound is present in an amount less than 0.5% (w/w, w/v, v/v, or % by dry weight) of the composition.

53. The composition of claim 50, wherein the second compound is selected from a compound in Table 2.
